(19) United States
(12) Patent Application Publication
(10) Pub. No.: US 2023/0279090 A1
(43) Pub. Date: Sep. 7, 2023

(54) NON-COVALENT PROTEIN-HYALURONAN CONJUGATES FOR LONG-ACTING OCULAR DELIVERY

(71) Applicants: Genentech, Inc., South San Francisco, CA (US); HOFFMANN-LA ROCHE INC., Little Falls, NJ (US)

(72) Inventors: Stefan Dengl, Geretsried (DE); Rami Hannoush, San Mateo, CA (US); Simon Theodor Hansen, San Francisco, CA (US); Peter Michael Huelsmann, Habach (DE); Robert Franklin Kelley, Petaluma, CA (US); Hubert Kettenberger, Muenchen (DE); Shrenik Chetan Mehta, San Carlos, CA (US); Devin Brent Tesar, San Francisco, CA (US)

(21) Appl. No.: 18/301,187

(22) Filed: Apr. 14, 2023

Related U.S. Application Data
(63) Continuation of application No. PCT/EP2021/078433, filed on Oct. 14, 2021.
(60) Provisional application No. 63/250,782, filed on Sep. 30, 2021.
(60) Provisional application No. 63/092,251, filed on Oct. 15, 2020.

(51) Int. Cl. C07K 16/22; C07K 14/705; A61P 27/02

(52) U.S. Cl. CPC C07K 16/22; A61P 27/02; C07K 14/70578; C07K 14/70585

(57) ABSTRACT

A conjugate may comprise a first component capable of binding to a therapeutic target in the eye, one or more second component(s) capable of binding to hyaluronan, and one or more third component(s) comprising hyaluronan, wherein each second component is covalently bound to the first component and non-covalently bound to a third component, a composition comprising the conjugate for use as a medicament or for use in the treatment of an eye disease and a method of treating an eye disease in a subject. Additionally, a therapeutic molecule targeted to a tissue in a patient may comprises a hyaluronic acid binding moiety and a therapeutically active agent, wherein the hyaluronic acid binding moiety comprises at least two link domains of Versican.

### CROSS-REFERENCE TO RELATED APPLICATIONS

This application is a continuation of International Patent Application No. PCT/EP21/78433 filed on Oct. 14, 2021, which claims the right of priority to U.S. Provisional Appl. Ser. No. 63/092,251, filed Oct. 15, 2020, and to U.S. Provisional Appl. Ser. No. 63/250,782, filed Sep. 30, 2021, which are commonly owned with the present application and the entire contents of each of which are hereby expressly incorporated by reference in their entirety as though fully set forth herein.

### SEQUENCE LISTING

The instant application contains a Sequence Listing which has been submitted via EFS-Web. The content of the file named “048893-584C01US_SequenceListing_ST26.xml”, which was created on Apr. 14, 2023 and is 200,704 bytes in size, is hereby incorporated by reference in its entirety.

### FIELD

Long-acting therapeutics and methods of treatment employing fusion proteins that bind to hyaluronan and fusion protein-hyaluronan conjugates.

### BACKGROUND

Intravitreal (IVT) injections are commonly used to administer medications to treat a variety of eye conditions. IVT injections allow for direct application of a drug into the posterior eye, thus eliminating the barriers that are common with topical and systemic administration. Direct application of a drug in this way allows for higher intraocular bioavailability of the drug in posterior segment tissues, which yields more efficacious treatment of posterior eye diseases. Stewart, M.W., Expert Opinion on Drug Metabolism & Toxicology, 14(1):5-7 (2018). Examples of common conditions that are treated via IVT injections include age-related macular degeneration (AMD), diabetic retinopathy, retinal vein occlusion, and eye infections (such as endophthalmitis and retinitis). The Foundation of American Society of Retina Specialists, asrs.org/patients/retinal-diseases/33/IVT-injections (2017).

Despite encouraging results in halting disease and improving vision, IVT injections are uncomfortable and expensive, and require a retinal specialist to perform them. IVT injections are known to cause adverse effects in some patients such as infection, inflammation, bleeding into the vitreous, increased presence of floaters in the eye, increased sensitivity to light, decreased vision, and retinal detachment. The Foundation of American Society of Retina Specialists, asrs.org/patients/retinal-diseases/33/IVT-injections (2017). IVT injections may also be associated with infectious endophthalmitis, sterile intraocular inflammation, rhegmatogenous retinal detachment, increased intraocular pressure and ocular hemorrhage. Id. Ocular long-acting delivery technologies can circumvent the need for repeated injections of a drug, which lend to improved patient compliance and clinical outcome. Methods and compositions that extend drug half-life in the vitreous humor (e.g., the ability to maintain a drug reservoir, a low turnover rate in the eye, a low retention-target mediated clearance, and/or seemingly stable properties in aged population) promote slow release of the drug from injection site to target site, enabling the use of higher doses and reducing the number of required injections.

The vitreal half-life of therapeutic molecules can be extended by binding the therapeutic molecule to hyaluronan (HA) as an alternative to encapsulation or chemical modifications with polymers. Cromwell, S et al., Invest. Ophthalmol. Vis. Sci. 59(9):235 (2018); Ghosh, J.G. et al., Nature Communications, 8:14837, doi:10.1038/ncomms14837 (2017); Stewart, M.W., Expert Opinion on Drug Metabolism & Toxicology, 14(1):5-7 (2018). In a particular example, long-acting anti-VEGF antibodies were individually fused to HA binding domains (HABDs) of human tumor necrosis factor (TNF)-stimulated gene 6 protein (TSG-6). Ghosh, J.G. et al., Nature Communications, 8:14837, doi:10.1038/ncomms14837 (2017). The fusion proteins demonstrated the following improvements relative to unmodified anti-VEGF antibodies: (1) a 3 to 4-fold increase in half-life; and (2) the ability to attenuate VEGF-induced retinal changes in animal models of neovascular retinal disease over a period that is 3-4-fold longer. Ghosh, J.G. et al., Nature Communications, 8:14837, doi:10.1038/ncomms14837 (2017). A drug candidate comprising a fusion of a long-acting anti-VEGF antibody with TSG-6, LMG324, was advanced into clinical trials for evaluation of the safety and tolerability of single ascending doses to determine the maximum tolerated dose (MTD) in neovascular age-related macular degeneration (nvAMD). clinicaltrials.gov/ct2/show/NCT02398500 (2019). Unfortunately, the trials were halted due to severe adverse events which included vitreous floaters, inflammation, and posterior vitreous detachment.

Chemical conjugation of antibody fragments with hyaluronan (HA) may reduce the diffusion rate of the drug from the vitreous. However, this approach requires chemical activation of HA; the use of non-natural linkers may lead to non-natural metabolites of activated HA in a subject.

The inventors found that the above-mentioned drawbacks can be avoided by providing a conjugate comprising: (1) a first component capable of binding to a therapeutic target in the eye, (2) one or more second components capable of binding to HA, and (3) one or more third components comprising HA; wherein each second component is (a) covalently bound to the first component and (b) noncovalently bound to a third component. Unlike the anti-VEGF antibody and TSG-6 fusion protein, LMG324, described above, the second component capable of binding to HA is pre-complexed with HA.

The present application discloses materials and methods to increase ocular retention of therapeutic molecules comprising fusion proteins that are capable of binding hyaluronan (HA). In some embodiments, a fusion protein comprises: (1) a first component capable of binding to a therapeutic target in the eye, and (2) one or more second components capable of binding to HA; wherein each second component is covalently bound to the first component.

The present application also discloses conjugates wherein said fusion proteins further comprise one or more third components comprising HA, wherein each second component is further non-covalently bound to the third component. Further, the second component capable of binding to HA may be pre-complexed with HA. The conjugates are compatible with vitreous and have binding affinity for HA. The materials and methods provide a platform technology for improved long-acting drug design.

### SUMMARY

The materials and methods relate to therapeutic molecules and conjugates thereof capable of binding to a therapeutic target in the eye and capable of binding to hyaluronan. The following items, aspects, and embodiments are provided.

Item 1 is a therapeutic molecule comprising: (a) first component capable of binding to a therapeutic target in the eye, (b) one or more second components capable of binding to hyaluronan, wherein the one or more second components are covalently bound to the first component, and (c) optionally, one or more third components comprising hyaluronan, wherein, if present, the one or more third components are non-covalently bound to the one or more second components.

Item 2 is the therapeutic molecule of item 1, wherein the first component is a protein, a peptide, a receptor or fragment thereof, a ligand to a receptor, a darpin, a nucleic acid, an RNA, a DNA, or an aptamer.

Item 3 is the conjugate of item 1 or 2, wherein the first component is chosen from an antibody, antigen-binding fragment, particularly an antibody fragment, more particularly an antibody fragment lacking at least the Fc domain, especially wherein the fragment is or comprises an (Fab′)₂ fragment, Fab′ fragment, or Fab fragment, VhH fragment, scFv fragment, scFv-Fc fragment, and minibody, more especially a Fab fragment.

Item 4 is the therapeutic molecule of any of items 1 to 3, wherein the second component comprises a hyaluronan receptor CD44 (CD44) domain, a brain-specific link protein (BRAL1) domain, a tumor necrosis factor-stimulated gene-6 (TSG-6) domain, a Lymphatic Vessel Endothelial Hyaluronan Receptor-1 (LYVE-1) domain, or a Hyaluronic Acid Binding Protein (HABP) domain, an Aggrecan G1 (AG1) domain or a Versican G1 (VG1) domain.

Item 5 is the therapeutic molecule of any of items 1 to 4, wherein the conjugate comprises one second component or two second components that are identical to each other.

Item 6 is the therapeutic molecule of any of items 1 to 4, wherein the third component is a hyaluronan, wherein the hyaluronan (a) has a molecular weight (i) chosen from 3 kDa to 60 kDa, from 4 kDa to 30 kDa, from 5 kDa to 20 kDa, or from 400 Da to 200 kDa; (ii) of at least 2, 3, 4, 5, 6, 7, 8, or 9 kDa; or (iii) of at most 60, 50, 40, 30, 25, 20, or 15 kDa; (b) provides a molar excess of binding equivalents to the one or two second components; and (c) has a modification reducing degradation of the hyaluronan in the eye.

Item 7 is the therapeutic molecule of any of items 1 to 6, wherein the second component is capable of binding to hyaluronan with a K_{D} of 10 nM to 10 µM, 5 nM to 8 µM, or 100 nM to 5 µM.

Item 8 is the therapeutic molecule of any of items 1 to 7, wherein (a) the first and the second components are comprised in a fusion protein, particularly wherein the one or two second components are covalently bound to the N-terminus and/or the C-terminus of the first component, more particularly wherein the first component is an antibody or antigen-binding fragment and wherein the one or two second components are covalently bound to a C-terminus of the first component; and/or (b) the one or two second components are directly bound to the first component or bound indirectly to the first component via a linker, particularly a linker of at least 4 amino acids and/or at most 50 or at most 25 amino acids, more particularly a linker being (GxS)ₙ or (GxS)ₙGₘ with G = glycine, S = serine, (x = 3, n = 3, 4, 5 or 6, and m = 0, 1, 2 or 3) or (x = 4, n = 2, 3, 4 or 5 and m = 0, 1, 2 or 3).

Item 9 is the therapeutic molecule of any of items 1 to 8, wherein the therapeutic target is VEGF, C2, C3a, C3b, C5, C5a, Htra1, IL-33, Factor P, Factor D, EPO, EPOR, IL-1β, IL-17A, IL-10, TNFα, FGFR2, PDGF or ANG2, especially VEGF.

Item 10 is the therapeutic molecule of any of items 1 to 9, wherein (a) the first component is an antibody or antigen-binding fragment against VEGF, particularly an anti-VEGF Fab; and/or (b) each of the one or two second components comprise a CD44 domain or a TSG-6 domain or a VG1 domain; and/or (c) the third component is a hyaluronan of a molecular weight of from 5 kDa to 20 kDa.

Item 11 is the therapeutic molecule of any one of items 1 to 10, wherein (i) the first component is an anti-VEGF antibody or antigen-binding fragment, the one or two second components comprise a CD44 domain, and the third component is a hyaluronan of a molecular weight of from 5 kDa to 20 kDa; (ii) the first component is an anti-VEGF antibody or antigen-binding fragment, the one or two second components comprise a TSG-6 domain, and the third component is a hyaluronan of a molecular weight of from 5 kDa to 20 kDa; or (iii) the first component is an anti-VEGF antibody or antigen-binding fragment, the one or two second components comprise a VG1 domain, and the third component is a hyaluronan of a molecular weight of from 5 kDa to 20 kDa.

Item 12 is the conjugate of any of items 1 to 11, wherein (a) the first component comprises (i) the VH domain of SEQ ID NO: 97, 99, 105, 109, or 144; and (ii) the VL domain of SEQ ID NO: 98, 100, 106, 110, or 115; and (b) the second component comprises SEQ ID NO: 2.

Item 13 is the conjugate of any of items 1 to 11, wherein (a) the first component comprises (i) the VH domain of SEQ ID NO: 97, 99, 105, 109, or 144; and (ii) the VL domain of SEQ ID NO: 98, 100, 106, 110, or 115; and (b) the second component comprises SEQ ID NO: 4.

Item 14 is the conjugate of any of items 1 to 11, wherein (a) the first component comprises (i) the VH domain of SEQ ID NO: 97, 99, 105, 109, or 144; and (ii) the VL domain of SEQ ID NO: 98, 100, 106, 110, or 115; and (b) the second component comprises SEQ ID NO: 86, 60, 32, or 29.

Item 15 is the therapeutic molecule of any one of claims 1 to 11, wherein the second components comprise at least two link domains of Versican.

Item 16 is the therapeutic molecule of item 15, wherein the second components comprise at least two link domains of Versican that are bound to hyaluronan.

Item 17 is the therapeutic molecule of any one of items 1-22, wherein the hyaluronan allows for a ratio of hyaluronan to therapeutic molecule that ranges from 1.5:1 to 1:1.

Item 18 is the therapeutic molecule of any one of items 14-17, wherein the second component comprises at least 90%, 91%, 92%, 93%, 94%, 95%, 96%, 97%, 98%, 99%, or 100% identity to SEQ ID NO: 86, 60, 32, or 29.

Item 19 is the therapeutic molecule of any one of items 14-18, wherein the second component comprises at least 95% identity to SEQ ID NO: 86, 60, 32, or 29.

Item 20 is the therapeutic molecule of any one of items 14-19, wherein the second component comprises at least 1, at least 2, at least 3, at least 4, or at least 5 mutations.

Item 21 is the therapeutic molecule of any one of items 14-20, wherein the second component comprises 1 to 3 mutations, wherein the 1 to 3 mutations comprise single amino acid substitutions, double amino acid substitutions, and truncations.

Item 22 is the therapeutic molecule of any one of items 14-21, wherein the second component comprises 1 to 5 mutations, wherein the 1 to 5 mutations comprise single amino acid substitutions, double amino acid substitutions, and truncations.

Item 23 is the therapeutic molecule of any one of items 14-22, wherein the second component has a truncation mutation relative to SEQ ID NO: 29.

Item 24 is the therapeutic molecule of item 23, wherein the truncation mutation comprises a truncation from 1 to 129 amino acids on the N-terminus.

Item 25 is the therapeutic molecule of any one of items 14-24, wherein the second component is a truncated sequence wherein the Ig domain of wild type Versican is absent.

Item 26 is the therapeutic molecule of any one of items 14-25, wherein the second component comprises at least one of the following amino acids relative to SEQ ID NO: 29: R160, Y161, E194, D197, Y208, R214, Y230, F261, D295, and R233.

Item 27 is the therapeutic molecule of any one of items 14-26, wherein the second component comprises 2, 3, 4, 5, 6, 7, 8, 9, or 10 of the following amino acids relative to SEQ ID NO: 29: R160, Y161, E194, D197, Y208, R214, Y230, F261, D295, and R233.

Item 28 is the therapeutic molecule of any one of items 14-27, wherein the second component comprises a mutation in at least one of the following positions relative to SEQ ID NO: 29: R160, Y161, E194, D197, Y208, R214, M222, Y230, R233, K260, F261, D295, Y296, H306, R312, L325, Y326, and R327.

Item 29 is the therapeutic molecule of any one of items 14-28, wherein the second component comprises a mutation in 2, 3, 4, 5, 6, 7, 8, 9, 10, 11, 12, 13, 14, 15, 16, 17, or 18 of the following positions relative to SEQ ID NO: 29: R160, Y161, E194, D197, Y208, R214, M222, Y230, R233, K260, F261, D295, Y296, H306, R312, L325, Y326, and R327.

Item 30 is the therapeutic molecule of any one of items 14-29, wherein the second component comprises a mutation in 2, 3, 4, 5, or 6 of the following positions relative to SEQ ID NO: 29: R160, Y161, E194, D197, Y208, R214, M222, Y230, R233, K260, F261, D295, Y296, H306, R312, L325, Y326, and R327.

Item 31 is the therapeutic molecule of any one of items 14-30, wherein the second component comprises at least one of the following mutations relative to SEQ ID NO: 29: R160A, Y161A, D197A, D197S, Y208A, Y208F, R214K, M222A, Y230A, Y230F, R233A, K260A, K260R, F261Y, KF260RY, D295A, D295S, Y296A, Y296F, DY295SF, H306A, R312A, L325A, Y326A, R327A, and LYR325LFK.

Item 32 is the therapeutic molecule of any one of items 14-31, wherein the second component comprises at least one of Y208A and H306A.

Item 33 is the therapeutic molecule of any one of items 14-32, wherein the second component comprises at least 2, 3, 4, 5, 6, 7, 8, 9, 10, 11, 12, 13, 14, 15, 16, or 17 of the following mutations relative to SEQ ID NO: 29: R160A, Y161A, D197A, D197S, Y208A, Y208F, R214K, M222A, Y230A, Y230F, R233A, K260A, K260R, F261Y, KF260RY, D295A, D295S, Y296A, Y296F, DY295SF, H306A, R312A, L325A, Y326A, R327A, and LYR325LFK.

Item 34 is the therapeutic molecule of any one of items 14-33, wherein the second component comprises at least 2, 3, 4, 5, or 6 of the following mutations relative to SEQ ID NO: 29: R160A, Y161A, D197A, D197S, Y208A, Y208F, R214K, M222A, Y230A, Y230F, R233A, K260A, K260R, F261Y, KF260RY, D295A, D295S, Y296A, Y296F, DY295SF, H306A, R312A, L325A, Y326A, R327A, and LYR325LFK.

Item 35 is the therapeutic molecule of any one items 14 or 18, wherein the second component is SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or SEQ ID NO: 59.

Item 36 is the therapeutic molecule of any one of items 1-35, wherein the first component comprises an oligopeptide, protein, or a nucleic acid.

Item 37 is the therapeutic molecule of any one of items 1-36, wherein the first component comprises a therapeutic drug, an antibody, an antigen-binding fragment, an enzyme, a growth factor, an oligopeptide, a cysteine knot peptide, a growth factor, an antisense oligonucleotide, a locked nucleic acid, or an aptamer.

Item 38 is the therapeutic molecule of item 37, wherein the cysteine knot peptide is at least 90%, 91%, 92%, 93%, 94%, 95%, 96%, 97%, 98%, 99%, or 100% identical to SEQ ID NO: 92.

Item 39 is the therapeutic molecule of item 37, wherein the growth factor comprises fibroblasts growth factors, platelet-derived growth factors, nerve growth factor (NGF), VEGF, fibroblast growth factor (FGF), and insulin-like growth factor-I (IGF-I).

Item 40 is the therapeutic molecule of any one of items 1-39, wherein the first component binds VEGF.

Item 41 is the therapeutic molecule of item 40, wherein the first component that binds VEGF comprises ranibizumab, aflibercept, brolucizumab-dbll, and bevacizumab.

Item 42 is the therapeutic molecule of item 37, wherein the aptamer is pegylated.

Item 43 is the therapeutic molecule of any one of items 37 or 42, wherein the aptamer is Macugen®.

Item 44 is the therapeutic molecule of any one of items 1-43, wherein the linker comprises GGGGS (SEQ ID NO: 27) or a multimer thereof, more especially (GGGGS)3 (SEQ ID NO: 28).

Item 45 is the therapeutic molecule of any one of items 1-42, wherein the linker comprises GSGSGSGSGSGSGSGSGSGS (SEQ ID NO: 95).

Item 46 is the therapeutic molecule of item 45, wherein the cysteine knot peptide and the one or two second components are linked via a linker comprising the sequence GSGSGSGSGSGSGSGSGSGS (SEQ ID NO: 95).

Item 47 is the therapeutic molecule of item 45 or 46, wherein the sequence comprises (a) an anti-VEGF antigen-binding fragment; and (b) at least 90%, 91%, 92%, 93%, 94%, 95%, 96%, 97%, 98%, 99%, or 100% sequence identity with SEQ ID NO: 93 or SEQ ID NO: 94.

Item 48 is a composition for use as a medicament, the composition comprising the therapeutic molecule of any one of items 1 to 47 and optionally a pharmaceutically acceptable excipient, diluent, or carrier.

Item 49 is a composition for use in the treatment of an eye disease or a brain disease, the composition comprising the conjugate of any one of items 1 to 47 and optionally a pharmaceutically acceptable excipient, diluent or carrier.

Item 50 is the composition for use of item 49, formulated for intraocular delivery, particularly intravitreal injection.

Item 51 is the composition for use of any of items 48 to 50, wherein (a) the composition is to be administered at most every three months, particularly at most every four months, more particularly at most every six months; and/or (b) the elimination half-life of the first component in the conjugate is extended at least 3-fold, at least 4-fold or at least 5-fold as compared to the unconjugated first component.

Item 52 is the composition for use of any of items 48 to 51, wherein the eye disease is age-related macular degeneration (AMD), particularly wet AMD or neovascular AMD, diabetic macular edema (DME), diabetic retinopathy (DR), particularly proliferative DR or non-proliferative DR, retinal vein occlusion (RVO) or geographic atrophy (GA).

Item 53 is a method of treating an eye disease in a subject, the method comprising administering to the subject the therapeutic molecule of any of items 1 to 47 or a composition as defined in any of items 48 to 52.

Item 54 is a method of delivery for a therapeutic molecule targeted to a tissue in a patient comprising administering the therapeutic molecule of any one of items 1 to 47 or the composition of any one of items 48 to 52 to the patient. and allowing the therapeutic molecule to provide long-acting delivery of the first component to the target tissue.

Item 55 is the method of item 54, further comprising binding the therapeutic molecule to hyaluronan before the administering step.

Item 56 is the method of item 55, further comprising mixing a first solution comprising the therapeutic molecule and a second solution comprising the hyaluronan.

Item 57 is the method of item 56, wherein the mixing comprises a vessel.

Item 58 is the method of item 57, wherein the vessel is a two-compartment syringe.

Item 59 is the method of any one of items 56 to 58, wherein the mixing produces a therapeutic molecule bound to hyaluronan that is ready for administering to a subject.

Item 60 is the method of any one of items 54 to 59, wherein the administering step is a single injection.

Item 61 is the method of any one of items 54 to 60, wherein the target tissue comprises the eye or the brain.

Item 62 is the method of any one of items 54 to 61, wherein the therapeutic molecule provides improved vitreous compatibility, longer vitreous residence time, longer vitreous half-life, and/or improved duration of pharmacological effect in comparison to unmodified biologically active agent.

Aspect 63 is a conjugate comprising (a) a first component capable of binding to a therapeutic target in the eye; (b) one or more second component(s) capable of binding to hyaluronan; and (c) one or more third component(s) comprising hyaluronan, (d) wherein each second component is covalently bound to the first component and non-covalently bound to a third component.

Aspect 64 is the conjugate of aspect 63, wherein the first component is a protein, a peptide, a receptor or fragment thereof, a ligand to a receptor, a darpin, a nucleic acid, a RNA, a DNA or an aptamer.

Aspect 65 is the conjugate of aspect 63 or 64, wherein the first component is an antibody, or antigen binding antibody fragment, particularly an antibody fragment, more particularly an antibody fragment lacking at least the Fc domain, especially wherein the fragment is or comprises a (Fab′)2 fragment, a Fab′ fragment, or a Fab fragment, more especially a Fab fragment.

Aspect 66 is the conjugate of any of aspects 63-65, wherein the second component comprises a hyaluronan receptor CD44 (CD44) domain, a brain-specific link protein (BRAL1) domain, a tumor necrosis factor-stimulated gene-6 (TSG-6) domain, a Lymphatic Vessel Endothelial Hyaluronan Receptor-1 (LYVE-1) domain, or a Hyaluronic Acid Binding Protein (HABP) domain, an aggrecan G1 (AG1) domain or a versican G1 (VG1) domain.

Aspect 67 is the conjugate of any of aspects 63-66, wherein the conjugate comprises one or two second components, particularly two identical second components.

Aspect 68 is the conjugate of any of aspects 63-66, wherein the third component is a hyaluronan, wherein the hyaluronan (a) has a molecular weight of from 3 kDa to 60 kDa, particularly of from 4 kDa to 30 kDa, more particularly of from 5 kDa to 20 kDa; and/or (b) has a molecular weight of at least 2, 3, 4, 5, 6, 7, 8, or 9 kDa; and/or (c) has a molecular weight of at most 60, 50, 40, 30, 25, 20, or 15 kDa; and/or (d) has a modification reducing degradation of the hyaluronan in the eye.

Aspect 69 is the conjugate of any of aspects 63-67, wherein (a) the first and the second components are comprised in a fusion protein, particularly wherein one or two of the second component(s) is/are covalently bound to the N terminus and/or the C terminus of the first component, more particularly wherein the first component is an antibody or antigen binding antibody fragment and wherein one or two of the second component(s) is/are covalently bound to a C terminus of the first component; and/or (b) the second component(s) is/are directly bound to the first component or bound indirectly to the first component via a linker, particularly a linker of at least 4 amino acids and/or at most 50 or at most 25 amino acids, more particularly a linker being (GxS)n or (GxS)nGm with G = glycine, S = serine, (x = 3, n = 3, 4, 5 or 6, and m = 0, 1, 2 or 3) or (x = 4, n = 2, 3, 4 or 5 and m = 0, 1, 2 or 3).

Aspect 70 is the conjugate of any of aspects 63-69, wherein the therapeutic target is VEGF, C5, Factor P, Factor D, EPO, EPOR, IL-1β, IL-17A, IL-10, TNF□, FGFR2, PDGF or ANG2, especially VEGF.

Aspect 71 is the conjugate of any of aspects 63-70, wherein (a) the first component is an antibody or antigen binding antibody fragment against VEGF, particularly a anti-VEGF Fab; and/or (b) each of the one or two second components comprises a CD44 domain or a TSG-6 domain or a VG1 domain; and/or (c) the third component is a hyaluronan of a molecular weight of from 5 kDa to 20 kDa, (d) particularly wherein (e) the first component is an anti-VEGF Fab and wherein each of the one or two second components comprises a CD44 domain and wherein the third component is a hyaluronan of a molecular weight of from 5 kDa to 20 kDa; or (f) the first component is an anti-VEGF Fab and wherein each of the one or two second components comprises a TSG-6 domain and wherein the third component is a hyaluronan of a molecular weight of from 5 kDa to 20 kDa; or (g) the first component is an anti-VEGF Fab and wherein each of the one or two second components comprises a VG1 domain and wherein the third component is a hyaluronan of a molecular weight of from 5 kDa to 20 kDa.

Aspect 72 is the conjugate of any of aspects 63-71, the first component is an antibody having the VH domain comprised in SEQ ID NO: 5 and the VL domain comprised in SEQ ID NO: 6 and the second component comprises or consists of SEQ ID NO: 4.

Aspect 73 is a composition for use as a medicament, the composition comprising the conjugate of any of aspects 63-72 and optionally a pharmaceutically acceptable excipient, diluent or carrier.

Aspect 74 is a composition for use in the treatment of an eye disease, the composition comprising the conjugate of any of aspects 63-73 and optionally a pharmaceutically acceptable excipient, diluent or carrier.

Aspect 75 is the composition for use of aspect 73 or 74, formulated for intraocular delivery, particularly intravitreal injection.

Aspect 76 is the composition for use of any of aspects 73-75, wherein (a) the composition is to be administered at most every three months, particularly at most every four months, more particularly at most every six months; and/or (b) the elimination half-life of the first component in the conjugate is extended at least 3-fold, at least 4-fold or at least 5-fold as compared to the unconjugated first component.

Aspect 77 is the composition for use of any of aspects 73-76, wherein the eye disease is age-related macular degeneration (AMD), particularly wet AMD or neovascular AMD, diabetic macular edema (DME), diabetic retinopathy (DR), particularly proliferative DR or non-proliferative DR, retinal vein occlusion (RVO) or geographic atrophy (GA).

Aspect 78 is a method of treating an eye disease in a subject, the method comprising administering to the subject the conjugate of any of aspects 63-72 or a composition as defined in any of aspects 73-77.

Embodiment 79 is a therapeutic molecule targeted to a tissue in a patient comprising a hyaluronan-binding domain and a therapeutically active agent, wherein the hyaluronan-binding domain comprises at least two link domains of Versican.

Embodiment 80 is a therapeutic molecule targeted to a tissue in a patient comprising a hyaluronan-binding domain and a therapeutically active agent, wherein the hyaluronan-binding domain comprises at least two link domains of Versican that are bound to hyaluronan via the HA-binding domain.

Embodiment 81 is the therapeutic molecule of embodiment 79 or 80, wherein the hyaluronan ranges from 400 Da to 200 kDa.

Embodiment 82 is the therapeutic molecule of embodiment 81, wherein the hyaluronan is at least 5 kDa.

Embodiment 83 is the therapeutic molecule of embodiment 81 or 82, wherein the hyaluronan is 10 kDa.

Embodiment 84 is the therapeutic molecule of any one of embodiments 79-83, wherein the hyaluronan provides a molar excess of binding equivalents to the link domains of Versican.

Embodiment 85 is the therapeutic molecule of any one of embodiments 79-84, wherein the hyaluronan allows for a ratio of hyaluronan to therapeutic molecule that ranges from 1.5:1 to 1:1.

Embodiment 83 is the therapeutic molecule of any one of embodiments 79-85, wherein the hyaluronan-binding domain has a K_{D} of 10 nM to 10 µM.

Embodiment 87 is the therapeutic molecule of any one of embodiments 79-86, wherein the hyaluronan-binding domain has a K_{D} of 5 nM to 8 µM.

Embodiment 88 is the therapeutic molecule of any one of embodiments 79-87, wherein the hyaluronan-binding domain has a K_{D} of 100 nM to 5 µM.

Embodiment 89 is the therapeutic molecule of any one of embodiments 79-88, wherein the hyaluronan-binding domain is at least 90%, 91%, 92%, 93%, 94%, 95%, 96%, 97%, 98%, 99%, or 100% identical to SEQ ID NO: 86, 60, 32, or 29.

Embodiment 90 is the therapeutic molecule of any one of embodiments 79-89, wherein the hyaluronan-binding domain is at least 95% identical to 86, 60, 32, or 29.

Embodiment 91 is the therapeutic molecule of any one of embodiments 79-90, wherein the hyaluronan-binding domain comprises at least 1, at least 2, at least 3, at least 4, or at least 5 mutations.

Embodiment 92 is the therapeutic molecule of any one of embodiments 79-91, wherein the hyaluronan-binding domain comprises 1 to 3 mutations, wherein the 1 to 3 mutations comprise single amino acid substitutions, double amino acid substitutions, and truncations.

Embodiment 93 is the therapeutic molecule of any one of embodiments 79-92, wherein the hyaluronan-binding domain comprises 1 to 5 mutations, wherein the 1 to 5 mutations comprise single amino acid substitutions, double amino acid substitutions, and truncations.

Embodiment 94 is the therapeutic molecule of any one of embodiments 79-93, wherein the hyaluronan-binding domain has a truncation mutation relative to SEQ ID NO: 29.

Embodiment 95 is the therapeutic molecule of embodiment 94, wherein the truncation mutation comprises a truncation from 1 to 129 amino acids on the N-terminus.

Embodiment 96 is the therapeutic molecule of any one of embodiments 79-95, wherein the hyaluronan-binding domain is a truncated sequence wherein the Ig domain of wild type Versican is absent.

Embodiment 97 is the therapeutic molecule of any one of embodiments 79-96, wherein the hyaluronan-binding domain comprises at least one of the following amino acids relative to SEQ ID NO: 29: R160, Y161, E194, D197, Y208, R214, Y230, F261, D295, and R233.

Embodiment 98 is the therapeutic molecule of any one of embodiments 79-97, wherein the hyaluronan-binding domain comprises 2, 3, 4, 5, 6, 7, 8, 9, or 10 of the following amino acids relative to SEQ ID NO: 29: R160, Y161, E194, D197, Y208, R214, Y230, F261, D295, and R233.

Embodiment 99 is the therapeutic molecule of any one of embodiments 79-98, wherein the hyaluronan-binding domain comprises a mutation in at least one of the following positions relative to SEQ ID NO: 29: R160, Y161, E194, D197, Y208, R214, M222, Y230, R233, K260, F261, D295, Y296, H306, R312, L325, Y326, and R327.

Embodiment 100 is the therapeutic molecule of any one of embodiments 79-99, wherein the hyaluronan-binding domain comprises a mutation in 2, 3, 4, 5, 6, 7, 8, 9, 10, 11, 12, 13, 14, 15, 16, 17, or 18 of the following positions relative to SEQ ID NO: 29: R160, Y161, E194, D197, Y208, R214, M222, Y230, R233, K260, F261, D295, Y296, H306, R312, L325, Y326, and R327.

Embodiment 101 is the therapeutic molecule of any one of embodiments 79-100, wherein the hyaluronan-binding domain comprises a mutation in 2, 3, 4, 5, or 6 of the following positions relative to SEQ ID NO: 29: R160, Y161, E194, D197, Y208, R214, M222, Y230, R233, K260, F261, D295, Y296, H306, R312, L325, Y326, and R327.

Embodiment 102 is the therapeutic molecule of any one of embodiments 79-101, wherein the hyaluronan-binding domain comprises at least one of the following mutations relative to SEQ ID NO: 29: R160A, Y161A, D197A, D197S, Y208A, Y208F, R214K, M222A, Y230A, Y230F, R233A, K260A, K260R, F261Y, KF260RY, D295A, D295S, Y296A, Y296F, DY295SF, H306A, R312A, L325A, Y326A, R327A, and LYR325LFK.

Embodiment 103 is the therapeutic molecule of any one of embodiments 79-102, wherein the hyaluronan-binding domain comprises at least one of Y208A and H306A.

Embodiment 104 is the therapeutic molecule of any one of embodiments 79-103, wherein the hyaluronan-binding domain comprises at least 2, 3, 4, 5, 6, 7, 8, 9, 10, 11, 12, 13, 14, 15, 16, or 17 of the following mutations relative to SEQ ID NO: 29: R160A, Y161A, D197A, D197S, Y208A, Y208F, R214K, M222A, Y230A, Y230F, R233A, K260A, K260R, F261Y, KF260RY, D295A, D295S, Y296A, Y296F, DY295SF, H306A, R312A, L325A, Y326A, R327A, and LYR325LFK.

Embodiment 105 is the therapeutic molecule of any one of embodiments 79-104, wherein the hyaluronan-binding domain comprises at least 2, 3, 4, 5, or 6 of the following mutations relative to SEQ ID NO: 29: R160A, Y161A, D197A, D197S, Y208A, Y208F, R214K, M222A, Y230A, Y230F, R233A, K260A, K260R, F261Y, KF260RY, D295A, D295S, Y296A, Y296F, DY295SF, H306A, R312A, L325A, Y326A, R327A, and LYR325LFK.

Embodiment 106 is the therapeutic molecule of any one embodiments 79-105, wherein the hyaluronan-binding domain is SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or SEQ ID NO: 59.

Embodiment 107 is the therapeutic molecule of any one of embodiments 79-106, wherein the therapeutically active agent comprises an oligopeptide, protein, or a nucleic acid.

Embodiment 108 is the therapeutic molecule of any one of embodiments 79-107, wherein the therapeutically active agent comprises an antibody, an antigen-binding fragment, a cysteine knot peptide, a growth factor, or an aptamer.

Embodiment 109 is the therapeutic molecule of embodiment 108, wherein the therapeutically active agent is capable of binding an antigen.

Embodiment 110 is the therapeutic molecule of embodiment 109, wherein the therapeutically active agent is capable of binding binds VEGF, HtrA1, IL-33, C5, Factor P, Factor D, EPO, EPOR, IL-1β, IL-17A, IL-10, TNFα, FGFR2, PDGF, or ANG2.

Embodiment 111 is the therapeutic molecule of any one of embodiments 109 or 110, wherein the therapeutically active agent is an antibody or an antigen-binding fragment thereof (including, but not limited to a Fab fragment, a F(ab′)2 fragment, a Fab′ fragment, VhH fragment, scFv fragment, scFv-Fc fragment, or minibody).

Embodiment 112 is the therapeutic molecule of any one of embodiments 109 or 110, wherein the therapeutically active agent is an oligopeptide or a protein.

Embodiment 113 is the therapeutic molecule of embodiment 102, wherein the oligopeptide or protein is a cysteine knot peptide or an enzyme.

Embodiment 114 is the therapeutic molecule of embodiment 103, wherein the cysteine knot peptide is at least 90%, 91%, 92%, 93%, 94%, 95%, 96%, 97%, 98%, 99%, or 100% identical to SEQ ID NO: 92.

Embodiment 115 is the therapeutic molecule of any one of embodiments 79-108, wherein the therapeutically active agent is a growth factor comprising fibroblasts growth factors, platelet-derived growth factors, nerve growth factor (NGF), VEGF, fibroblast growth factor (FGF), and insulin-like growth factor-I (IGF-I).

Embodiment 116 is the therapeutic molecule of embodiment 110, wherein the therapeutically active agent that binds VEGF comprises ranibizumab, aflibercept, brolucizumab-dbll, and bevacizumab.

Embodiment 117 is the therapeutic molecule of any one of embodiments 79-110, wherein the therapeutically active agent is a nucleic acid.

Embodiment 118 is the therapeutic molecule of embodiment 117, wherein the nucleic acid is an aptamer, an antisense oligonucleotide, and/or a locked nucleic acid.

Embodiment 119 is the therapeutic molecule of embodiment 118, wherein the aptamer binds VEGF.

Embodiment 120 is the therapeutic molecule of any one of embodiments 108, 118, or 119, wherein the aptamer is pegylated.

Embodiment 121 is the therapeutic molecule of any one of embodiments 108 or 118-120, wherein the aptamer is Macugen®.

Embodiment 122 is the therapeutic molecule of any one of embodiments 79-121, wherein the therapeutically active agent and the hyaluronan-binding domain are covalently linked via a linker.

Embodiment 123 is the therapeutic molecule of embodiment 122 wherein the linker is at least 4 amino acids.

Embodiment 124 is the therapeutic molecule of embodiment 122 or 123, wherein the linker is no longer than 50 amino acids.

Embodiment 125 is the therapeutic molecule of any one of embodiments 122-124, wherein the linker is from 4-25 amino acids.

Embodiment 126 is the therapeutic molecule of any one of embodiments 122-125, wherein the linker comprises (GxS)n or (GxS)nGm with G = glycine, S = serine, and (x = 3, n = 3, 4, 5, or 6, and m = 0, 1, 2, or 3) or (x = 4, n = 2, 3, 4, or 5 and m = 0, 1, 2, or 3).

Embodiment 127 is the therapeutic molecule of any one of embodiments 122-126, wherein the linker comprises GGGS (SEQ ID NO: 84) or a multimer thereof, more especially (GGGGS)3 (SEQ ID NO: 85).

Embodiment 128 is the therapeutic molecule of any one of embodiments 122-125, wherein the linker comprises (GxS)n with G = glycine, S = serine, and (n = 1, 2, 3, 4, 5, 6, 7, 8, 9, or 10).

Embodiment 129 is the therapeutic molecule of any one of embodiments 122-125 or 128, wherein the linker comprises GSGSGSGSGSGSGSGSGSGS (SEQ ID NO: 95).

Embodiment 130 is the therapeutic molecule of any one of embodiments 79-107, wherein the therapeutically active agent comprises an anti-VEGF antigen-binding moiety and a cysteine knot peptide.

Embodiment 131 is the therapeutic molecule of embodiment 130, wherein the cysteine knot peptide and the hyaluronan-binding domain are linked via a linker comprising the sequence GSGSGSGSGSGSGSGSGSGS (SEQ ID NO: 95).

Embodiment 132 is the therapeutic molecule of embodiment 130 or 131, wherein the sequence comprises (a) an anti-VEGF antigen-binding moiety; and (b) at least 90%, 91%, 92%, 93%, 94%, 95%, 96%, 97%, 98%, 99%, or 100% sequence identity with SEQ ID NO: 93, or SEQ ID NO: 94.

Embodiment 133 is the therapeutic molecule of any one of embodiments 79-132, wherein the hyaluronan-binding domain can bind non-covalently to hyaluronan.

Embodiment 134 is a method of delivery for a therapeutic molecule targeted to a tissue in a patient comprising administering the therapeutic molecule of any one of embodiments 79-133 to the patient and allowing the therapeutic molecule to provide long-acting delivery of the therapeutically active agent to the target tissue.

Embodiment 135 is the method of embodiment 134, further comprising binding the therapeutic molecule to hyaluronan before the administering step.

Embodiment 136 is the method of embodiment 135, further comprising mixing a first solution comprising the therapeutic molecule and a second solution comprising the hyaluronan.

Embodiment 137 is the method of embodiment 136, wherein the mixing comprises a vessel.

Embodiment 138 is the method of embodiment 137, wherein the vessel is a two-compartment syringe.

Embodiment 139 is the method of any one of embodiments 136-138, wherein the mixing produces a therapeutic molecule bound to hyaluronan that is ready for administering to a subject.

Embodiment 140 is the method of any one of embodiments 134-139, wherein the administering step is a single injection.

Embodiment 141 is the method of any one of embodiments 134-140, wherein the target tissue comprises the eye or the brain.

Embodiment 142 is the method of any one of embodiments 134-141, wherein the therapeutic molecule provides improved vitreous compatibility, longer vitreous residence time, longer vitreous half-life, and/or improved duration of pharmacological effect in comparison to unmodified therapeutically active agent.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows size exclusion chromatography (SEC; TSKgel UP-SW3000, 2 µm, 4.6×150 mm; running buffer 0.2 M KPh, 0.25 M KCl pH 6.2) of the Fab-hyaluronan-binding domain (Fab-HABD) fusion proteins VPDF-2xCD44, with and without pre-complexing with 10 kDa hyaluronan (HA). The Fab-HABDs were prepared as described in Example 1 and tested as described in Example 2.

FIGS. 2A-2B show vitreous pharmacokinetics (PK) of rabbit anti-c-Met Fab (RabFab) and rabbit anti-c-Met Fab-VG1 Fab-HABDs (RabFab-Fab-HABDs) following dose normalized intravitreal (IVT) injection in New Zealand White rabbits. FIG. 2A shows the amount of RabFab or RabFab-Fab-HABDs present in vitreous over time after IVT. Data is shown for RabFab-fusions, i.e., ¹²⁵I-RabFab-2xTSG6 (SEQ ID NOs: 15 and 16; 0.5 mg/eye) and RabFab-1xTSG6 (SEQ ID NOs: 13 and 14; 0.3 mg/eye), RabFab (SEQ ID NOs: 61 and 62; 0.3 mg/eye), and ¹²⁵I-Ranibizumab (¹²⁵I-Lucentis®) control (0.5 mg/eye). Data points are dose normalized. FIG. 2B shows vitreous pharmacokinetics as monitored by fluorophotometry for RabFab (0.15 mg/eye), or RabFab-2xTSG6 at 0.026 mg/eye, 0.15 mg/eye, or 2.5 mg/eye.

FIG. 3 shows a histopathology image for OS rabbit eye showing retinal degeneration at 4 days following IVT dosing of TSG6 (SEQ ID NO: 32).

FIGS. 4A-B show IVT pharmacokinetic (PK) profiles (mean concentration of drug over time) of VPDF (unmodified; FIG. 4A) and VPDF-2xCD44 + 10 kDa HA (FIG. 4B) in aqueous humor and in vitreous humor.

FIGS. 5A-C show different mixtures with pig vitreous. FIG. 5A shows pig vitreous mixed with unmodified anti-VEGF/anti-PDGF Fab fragment (VPDF), homogeneous (clear). FIG. 5B shows pig vitreous mixed with VPDF-2xCD44, inhomogeneous (precipitation). FIG. 5C shows pig vitreous mixed with VPDF-2xCD44 pre-complexes with 1% (w/v) HA 10 kDa, homogeneous (clear).

FIGS. 6A-6F show pig vitreous mixed with different concentrations of VPDF-2xCD44. FIG. 6A: 37.5 mg/mL VPDF-2xCD44. FIG. 6B: 9.4 mg/mL VPDF-2xCD44. FIG. 6C: 2.4 mg/mL VPDF-2xCD44. FIG. 6D: 0.6 mg/mL VPDF-2xCD44. FIG. 6E: 0.15 mg/mL VPDF-2xCD44. FIG. 6F: 0.04 mg/mL VPDF-2xCD44. +++ strong precipitation; ++ medium precipitation; + light precipitation; - clear vitreous.

FIGS. 7A-C show vitreous inhomogeneity in whole pig eye upon injection of indicated VPDF-2xCD44 sample. FIG. 7A: buffer control. FIG. 7B: uncomplexed VPDF-2xCD44. FIG. 7C: HA-complexed VPDF-2xCD44.

FIGS. 8A-B show the domain architecture of Versican and the amino acid sequence of link domains. Versican is endogenous to vitreous humor. FIG. 8A shows the Versican domains: VG1 domain, GAG attachment domain, and G3 domain. The VG1 domain (WT VG1; SEQ ID NO: 29) comprises an Ig-like domain followed by two link domains, i.e., Link1 and Link2, which are responsible for HA binding. FIG. 8B shows a sequence alignment of link domains which includes TSG6 LD (SEQ ID NO: 4), VG1 Link1 (SEQ ID NO: 30) and VG1 Link2 (SEQ ID NO: 31).

FIGS. 9A-B show precipitation of TSG6 but not WT VG1 in pig vitreous fluid. Turbidity was observed for mixing TSG6 (but not WT VG1) with 1:4 diluted (PBS) pig vitreous. Final concentrations of TSG6 and WT VG1 in vitreous were about 1 mg/mL. FIG. 9A shows TSG6 vs. control - pellet was observed upon centrifugation. FIG. 9B shows WT VG1 vs. control - no pellet was observed upon centrifugation.

FIGS. 10A-B show that RabFab-TSG6 precipitates in pig vitreous whereas RabFab-VG1 does not. TSG6 or VG1 are each recombinantly attached to RabFab and conjugated to Alexa488 via N-hydroxysuccinimide (NHS) primary amine-labeling chemistry. FIG. 10A shows RabFab-TSG6. FIG. 10B shows RabFab-VG1.

FIGS. 11A-C show that VG1 and RabFab-VG1 do not precipitate in rabbit vitreous fluid. FIG. 11A shows VG1 at ~40 g/L. FIG. 11B shows RabFab-VG1 at ~40 g/L. FIG. 11C shows RabFab-VG1 + 10 kDa HA at ~17 g/L. No precipitation was observed in any condition.

FIG. 12 shows fluorescence correlations spectroscopy (FCS) measurements of VG1 interaction with vitreous fluid ex vivo. Measurements that show slow diffusion indicate that the proteins interact with vitreous fluid while fast diffusion indicates that they do not. Dilution factors for vitreous fluid are shown at the top of the heatmap - the left-most column shows undiluted control/sample; the rightmost column shows phosphate-buffered saline (PBS), pH 7.4; and the columns in between show increasing dilution factors from left to right. Measurements for non-binding controls are shown in the two top rows. Measurements for the following samples are shown in rows 3-8: free VG1, PigFab-VG1, PigFab-VG1 + 10 kDa HA (1:1), free VG1, RabFab-VG1, and RabFab-VG1 + 10 kDa HA (1:1). The non-binding controls showed fastest diffusion (FIG. 12, rows 1 and 2). While all samples showed significant retarded diffusion relative to the controls, free VG1, PigFab-VG1, and RabFab-VG1 showed retarded diffusion until the vitreous was diluted greater than 6,000-fold (FIG. 12, rows 3, 4, 6, and 7; from undiluted to dilution factor 6,561). Slow diffusion was observed for samples co-formulated with 10 kDa HA, but the effect went away when dilution factor was greater than 729-fold (FIG. 12, row 5: PigFab-VG1+10 kDa HA (1:1), and row 8: RabFab-VG1+10 kDa HA (1:1); from dilution factor 729 to PBS). These results indicate that VG1 can interact with endogenous HA.

FIG. 13 shows thermal stress (i.e., protein stability) analysis for anti-HtrA1-VG1 at 37° C. T0 = no incubation control. T4wk = after 4 weeks of incubation.

FIG. 14 shows mean concentrations of pigFab-VG1 in pig aqueous humor. Concentrations were measured by mass spectrometry following IVT injection of 1.8 mg of pigFab-VG1 alone or pigFab-VG1 pre-complexed with equal mass concentration of 10 kDa HA. Mean values from several animals are shown with the error bars indicating standard deviation.

FIG. 15 shows percent inhibition of neovascularization by VPDF VG1 in rat laser-induced choroidal neovascularization (rat laser CNV).

FIGS. 16A-C show histopathology of rabbit eyes treated with test articles at 30 days post treatment. FIG. 16A shows WT VG1, FIG. 16B shows RabFab-VG1, and FIG. 16C shows RabFab-VG1 with HA.

FIGS. 17A-B show brain levels after intracerebroventricular injection in mice. FIG. 17A shows amounts protein retained in the brain over time. FIG. 17B shows exposure levels in the brain as measured by area-under-the-curve (AUC). ** indicates p<0.01, and *** indicates p<0.001, for comparison between groups. Anti-gD = anti-herpes simplex virus-1 glycoprotein D. BRD = anti-gD Fab-VG1.

FIG. 18 shows the crystal structure of WT VG1 and HA conjugate. The Ig domain of VG1 appears at the top of the figure, with the Link1 domain to the right on the bottom of the figure and the Link2 domain on the left of the bottom of the figure. The binding of HA is shown with the smaller HA molecule on the lower right side of the VG1 molecule.

FIG. 19 shows alignment of VG1 variants SEQ ID NOs: 29, 33-59. The first 20 amino acids on the N-terminus is the Versican signal sequence (SEQ ID NO: 123; shown with *). The boxed amino acids are conserved residues. All these proteins were produced with a C-terminal His-tag for purification.

### DESCRIPTION OF THE SEQUENCES

Table 1 provides a listing of certain sequences referenced herein. The amino acid sequences provided are from N-terminus to C-terminus.

TABLE 1

Description of the Sequences

| Description | Sequences | SEQ ID NO |
|---|---|---|
| Human CD44 full-length sequence | MDKFWWHAAWGLCLVPLSLAQIDLNITCRFAGVFHVEKNGRYSISRTEAADLCKAFNSTLPT MAQMEKALSIGFETCRYGFIEGHVVIPRIHPNSICAANNTGVYILTSNTSQYDTYCFNASAP PEEDCTSVTDLPNAFDGPITITIVNRDGTRYVQKGEYRTNPEDIYPSNPTDDDVSSGSSSER SSTSGGYIFYTFSTVHPIPDEDSPWITDSTDRIPATTLMSTSATATETATKRQETWDWFSWL FLPSESKNHLHTTTQMAGTSSNTISAGWEPNEENEDERDRHLSFSGSGIDDDEDFISSTIST TPRAFDHTKQNQDWTQWNPSHSNPEVLLQTTTRMTDVDRNGTTAYEGNWNPEAHPPLIHHEH HEEEETPHSTSTIQATPSSTTEETATQKEQWFGNRWHEGYRQTPKEDSHSTTGTAAASAHTS HPMQGRTTPSPEDSSWTDFFNPISHPMGRGHQAGRRMDMDSSHSITLQPTANPNTGLVEDLD RTGPLSMTTQQSNSQSFSTSHEGLEEDKDHPTTSTLTSSNRNDVTGGRRDPNHSEGSTTLLE GYTSHYPHTKESRTFIPVTSAKTGSFGVTAVTVGDSNSNVNRSLSGDQDTFHPSGGSHTTHG SESDGHSHGSQEGGANTTSGPIRTPQIPEWLIILASLLALALILAVCIAVNSRRRCGQKKKL VINSGNGAVEDRKPSGLNGEASKSQEMVHLVNKESSETPDQFMTADETRNLQNVDMKIGV | 1 |
| CD44 HA binding-domain sequence used in Fab-HABDs | AQIDLNITCRFAGVFHVEKNGRYSISRTEAADLCKAFNSTLPTMAQMEKALSIGFETCRYGF IEGHVVIPRIHPNSICAANNTGVYILTYNTSQYDTYCFNASAPPEEDCTSVTDLPNAFDGPI TITIVNRDGTRYVQKGEYRTNPEDIY | 2 |
| TNFAIP6; full-length TSG-6 | MIILIYLFLLLWEDTQGWGFKDGIFHNSIWLERAAGVYHREARSGKYKLTYAEAKAVCEFEG GHLATYKQLEAARKIGFHVCAAGWMAKGRVGYPIVKPGPNCGFGKTGIIDYGIRLNRSERWD AYCYNPHAKECGGVFTDPKQIFKSPGFPNEYEDNQICYWHIRLKYGQRIHLSFLDFDLEDDP | 3 |
| | GCLADYVEIYDSYDDVHGFVGRYCGDELPDDIISTGNVMTLKFLSDASVTAGGFQIKYVAMD PVSKSSQGKNTSTTSTGNKNFLAGRFSHL | |
| TSG-6 link domain (TSG6; 36-133) | GVYHREARSGKYKLTYAEAKAVCEFEGGHLATYKQLEAARKIGFHVCAAGWMAKGRVGYPIV KPGPNCGFGKTGIIDYGIRLNRSERWDAYCYNPHAKHHHHHH | 4 |
| VPDF-1xCD44 HC | DLQLVESGGGLVKPGGSLRLSCAADGWWFGYTDMSWVRQAPGKGLEWVGSISYKGGSTYYNT KFIGRFTISRDDDTNTLYLQMNSLRAEDTAVYYCARDDGYFDTWGQGTLVTVSSASTKGPSV FPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCGGGGSGGGGSGGGGSAQIDLNITCRFAGV FHVEKNGRYSISRTEAADLCKAFNSTLPTMAQMEKALSIGFETCRYGFIEGHVVIPRIHPNS ICAANNTGVYILTYNTSQYDTYCFNASAPPEEDCTSVTDLPNAFDGPITITIVNRDGTRYVQ KGEYRTNPEDIY | 5 |
| VPDF-1xCD44 LC | AIYMHQEPSSLSASVGDRVTITCHGSYWLSNYLAWYQQKPGKAPKLLIYDGKEREHGVPSRF SGSGSHEDYTLTISSLQPEDFATYYCQQYRYHPYTFGHGTKVEIKRTVAAPSVFIFPPSDEQ LKSGTASVVCLLNNFYPREAKVQWKVDNALQSGNSQESVTEQDSKDSTYSLSSTLTLSKADY EKHKVYACEVTHQGLSSPVTKSFNRGE | 6 |
| VPDF-2xCD44 HC | DLQLVESGGGLVKPGGSLRLSCAADGWWFGYTDMSWVRQAPGKGLEWVGSISYKGGSTYYNT KFIGRFTISRDDDTNTLYLQMNSLRAEDTAVYYCARDDGYFDTWGQGTLVTVSSASTKGPSV FPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCGGGGSGGGGSGGGGSAQIDLNITCRFAGV FHVEKNGRYSISRTEAADLCKAFNSTLPTMAQMEKALSIGFETCRYGFIEGHVVIPRIHPNS ICAANNTGVYILTYNTSQYDTYCFNASAPPEEDCTSVTDLPNAFDGPITITIVNRDGTRYVQ KGEYRTNPEDIY | 7 |
| VPDF-2xCD44 LC | AIYMHQEPSSLSASVGDRVTITCHGSYWLSNYLAWYQQKPGKAPKLLIYDGKEREHGVPSRF SGSGSHEDYTLTISSLQPEDFATYYCQQYRYHPYTFGHGTKVEIKRTVAAPSVFIFPPSDEQ LKSGTASVVCLLNNFYPREAKVQWKVDNALQSGNSQESVTEQDSKDSTYSLSSTLTLSKADY | 8 |
| | EKHKVYACEVTHQGLSSPVTKSFNRGECGGGGSGGGGSGGGGSAQIDLNITCRFAGVFHVEK NGRYSISRTEAADLCKAFNSTLPTMAQMEKALSIGFETCRYGFIEGHVVIPRIHPNSICAAN NTGVYILTYNTSQYDTYCFNASAPPEEDCTSVTDLPNAFDGPITITIVNRDGTRYVQKGEYR TNPEDIY | |
| Dig-1xCD44 HC | QVQLVESGGGLVKPGGSLRLSCAASGFTFSDYAMSWIRQAPGKGLEWVSSINIGATYIYYAD’ SVKGRFTISRDNAKNSLYLQMNSLRAEDTAVYYCARPGSPYEYDKAYYSMAYWGQGTTVTVS SASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCGGGGSGGGGSGGGGSAQIDL NITCRFAGVFHVEKNGRYSISRTEAADLCKAFNSTLPTMAQMEKALSIGFETCRYGFIEGHV VIPRIHPNSICAANNTGVYILTYNTSQYDTYCFNASAPPEEDCTSVTDLPNAFDGPITITIV NRDGTRYVQKGEYRTNPEDIY | 9 |
| Dig-1xCD44 LC | DIQMTQSPSSLSASVGDRVTITCRASQDIKNYLNWYQQKPGKAPKLLIYYSSTLLSGVPSRF SGSGSGTDFTLTISSLQPEDFATYYCQQSITLPPTFGGGTKVEIKRTVAAPSVFIFPPSDEQ LKSGTASVVCLLNNFYPREAKVQWKVDNALQSGNSQESVTEQDSKDSTYSLSSTLTLSKADY EKHKVYACEVTHQGLSSPVTKSFNRGEC | 10 |
| Dig-2xCD44 HC | QVQLVESGGGLVKPGGSLRLSCAASGFTFSDYAMSWIRQAPGKGLEWVSSINIGATYIYYAD SVKGRFTISRDNAKNSLYLQMNSLRAEDTAVYYCARPGSPYEYDKAYYSMAYWGQGTTVTVS SASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCGGGGSGGGGSGGGGSAQIDL NITCRFAGVFHVEKNGRYSISRTEAADLCKAFNSTLPTMAQMEKALSIGFETCRYGFIEGHV VIPRIHPNSICAANNTGVYILTYNTSQYDTYCFNASAPPEEDCTSVTDLPNAFDGPITITIV NRDGTRYVQKGEYRTNPEDIY | 11 |
| Dig-2xCD44 LC | DIQMTQSPSSLSASVGDRVTITCRASQDIKNYLNWYQQKPGKAPKLLIYYSSTLLSGVPSRF SGSGSGTDFTLTISSLQPEDFATYYCQQSITLPPTFGGGTKVEIKRTVAAPSVFIFPPSDEQ LKSGTASVVCLLNNFYPREAKVQWKVDNALQSGNSQESVTEQDSKDSTYSLSSTLTLSKADY EKHKVYACEVTHQGLSSPVTKSFNRGECGGGGSGGGGSGGGGSAQIDLNITCRFAGVFHVEK | 12 |
| | NGRYSISRTEAADLCKAFNSTLPTMAQMEKALSIGFETCRYGFIEGHVVIPRIHPNSICAAN NTGVYILTYNTSQYDTYCFNASAPPEEDCTSVTDLPNAFDGPITITIVNRDGTRYVQKGEYR TNPEDIY | |
| RabFab-1xTSG6 HC | QSLEESGGRLVTPGTPLTLTCTVSGFTISSYHMSWVRQAPGKGLEWIGIMRNTANIYYASWA KGRFTISKTSPTTVDLKMTSLTTEDTATYFCARGRPGDGALSLWGQGTLVTVSSGQPKAPSV FPLAPCCGDTPSSTVTLGCLVKGYLPEPVTVTWNSGTLTNGVRTFPSVRQSSGLYSLSSVVS VTSSSQPVTCNVAHPATNTKVDKTVAPSTCSKPTGGGGSGVYHREARSGKYKLTYAEAKAVC EFEGGHLATYKQLEAARKIGFHVCAAGWMAKGRVGYPIVKPGPNCGFGKTGIIDYGIRLNRS ERWDAYCYNPHAKHHHHHH | 13 |
| RabFab-1xTSG6 LC | ADVVMTQTPASVSAAVGGTVTIKCQASQSIGTALAWYQQKPGQPPKLLIYRTSTLESGVPSR FKGSGSGTDFTLTISDLECADAATYYCQSAYVSGGNIYTFGGGTEVVVKGDPVAPTVLIFPP AADQVATGTVTIVCVANKYFPDVTVTWEVDGTTQTTGIENSKTPQNSADCTYNLSSTLTLTS TQYNGHKEYTCKVTQGTTSVVQSFNRGDC | 14 |
| RabFab-2xTSG6 HC | QSLEESGGRLVTPGTPLTLTCTVSGFTISSYHMSWVRQAPGKGLEWIGIMRNTANIYYASWA KGRFTISKTSPTTVDLKMTSLTTEDTATYFCARGRPGDGALSLWGQGTLVTVSSGQPKAPSV FPLAPCCGDTPSSTVTLGCLVKGYLPEPVTVTWNSGTLTNGVRTFPSVRQSSGLYSLSSVVS VTSSSQPVTCNVAHPATNTKVDKTVAPSTCSKPTGGGGSGVYHREARSGKYKLTYAEAKAVC EFEGGHLATYKQLEAARKIGFHVCAAGWMAKGRVGYPIVKPGPNCGFGKTGIIDYGIRLNRS ERWDAYCYNPHAKHHHHHH | 15 |
| RabFab-2xTSG6 LC | ADVVMTQTPASVSAAVGGTVTIKCQASQSIGTALAWYQQKPGQPPKLLIYRTSTLESGVPSR FKGSGSGTDFTLTISDLECADAATYYCQSAYVSGGNIYTFGGGTEVVVKGDPVAPTVLIFPP AADQVATGTVTIVCVANKYFPDVTVTWEVDGTTQTTGIENSKTPQNSADCTYNLSSTLTLTS TQYNGHKEYTCKVTQGTTSVVQSFNRGDCGGGGSGVYHREARSGKYKLTYAEAKAVCEFEGG RLATYKQLEAARKIGFHVCAAGWMAKGRVGYPIVKPGSNCGFGKTGIIDYGIRLNRSERWDA YCYNPHAKDYKDDDDK | 16 |
| G6.31-1xTSG6 HC | EVQLVESGGGLVQPGGSLRLSCAASGFTISDYWIHWVRQAPGKGLEWVAGITPAGGYTYYAD SVKGRFTISADTSKNTAYLQMNSLRAEDTAVYYCARFVFFLPYAMDYWGQGTLVTVSSASTK GPSVFPLAPSSKSTSGGTAALGCLVKDYFPEPVTVSWNSGALTSGVHTFPAVLQSSGLYSLS SVVTVPSSSLGTQTYICNVNHKPSNTKVDKKVEPKSCDKTHTGGGGSGVYHREARSGKYKLT YAEAKAVCEFEGGHLATYKQLEAARKIGFHVCAAGWMAKGRVGYPIVKPGPNCGFGKTGIID YGIRLNRSERWDAYCYNPHAKHHHHHH | 17 |
| G6.31-1xTSG6 LC | DIQMTQSPSSLSASVGDRVTITCRASQDVSTAVAWYQQKPGKAPKLLIYSASFLYSGVPSRF SGSGSGTDFTLTISSLQPEDFATYYCQQGYGNPFTFGQGTKVEIKRTVAAPSVFIFPPSDEQ LKSGTASVVCLLNNFYPREAKVQWKVDNALQSGNSQESVTEQDSKDSTYSLSSTLTLSKADY EKHKVYACEVTHQGLSSPVTKSFNRGEC | 18 |
| G6.31-2xTSG6 HC | EVQLVESGGGLVQPGGSLRLSCAASGFTISDYWIHWVRQAPGKGLEWVAGITPAGGYTYYAD SVKGRFTISADTSKNTAYLQMNSLRAEDTAVYYCARFVFFLPYAMDYWGQGTLVTVSSASTK GPSVFPLAPSSKSTSGGTAALGCLVKDYFPEPVTVSWNSGALTSGVHTFPAVLQSSGLYSLS SVVTVPSSSLGTQTYICNVNHKPSNTKVDKKVEPKSCDKTHTGGGGSGVYHREARSGKYKLT YAEAKAVCEFEGGHLATYKQLEAARKIGFHVCAAGWMAKGRVGYPIVKPGPNCGFGKTGIID YGIRLNRSERWDAYCYNPHAKHHHHHH | 19 |
| G6.31-2xTSG6 LC | DIQMTQSPSSLSASVGDRVTITCRASQDVSTAVAWYQQKPGKAPKLLIYSASFLYSGVPSRF SGSGSGTDFTLTISSLQPEDFATYYCQQGYGNPFTFGQGTKVEIKRTVAAPSVFIFPPSDEQ LKSGTASVVCLLNNFYPREAKVQWKVDNALQSGNSQESVTEQDSKDSTYSLSSTLTLSKADY EKHKVYACEVTHQGLSSPVTKSFNRGECGGGGSGVYHREARSGKYKLTYAEAKAVCEFEGGH LATYKQLEAARKIGFHVCAAGWMAKGRVGYPIVKPGPNCGFGKTGIIDYGIRLNRSERWDAY CYNPHAKDYKDDDDK | 20 |
| NVS24-1xTSG6 (Lava12) HC | EVQLVESGGGLVQPGGSLRLSCTASGFSLTNYYYMTWVRQAPGKGLEWVGFIDPQNDPYYAT WAKGRFTISRDNSKNTLYLQMNSLRAEDTAVYYCAGGNHNSGWGLNIWGQGTLVTVSSASTK GPSVFPLAPSSKSTSGGTAALGCLVKDYFPEPVTVSWNSGALTSGVHTFPAVLQSSGLYSLS SVVTVPSSSLGTQTYICNVNHKPSNTKVDKRVEPKSCGSGGGGVYHREAISGKYYLTYAEAK | 21 |
| | AVCEFEGGHLATYKQLLAAQKIGFHVCAAGWMAKGRVGYPIVKPGPNCGFGKTGIIDYGIRL NRSERWDAYCYNPHA | |
| NVS24-1xTSG6 (Lava12) LC | EIVMTQSPSTLSASVGDRVIITCQASQKIHSWLAWYQQKPGKAPKLLIYQASKLAKGVPSRF SGSGSGAEFTLTISSLQPDDFATYYCQNVYLASTNGANFGQGTKLTVLKRTVAAPSVFIFPP SDEQLKSGTASVVCLLNNFYPREAKVQWKVDNALQSGNSQESVTEQDSKDSTYSLSSTLTLS KADYEKHKVYACEVTHQGLSSPVTKSFNRGEC | 22 |
| VPDF-1xTSG6 (Lava12) HC | DLQLVESGGGLVKPGGSLRLSCAADGWWFGYTDMSWVRQAPGKGLEWVGSISYKGGSTYYNT KFIGRFTISRDDDTNTLYLQMNSLRAEDTAVYYCARDDGYFDTWGQGTLVTVSSASTKGPSV FPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCDKTHTGGGGSGVYHREAISGKYYLTYAEA KAVCEFEGGHLATYKQLLAAQKIGFHVCAAGWMAKGRVGYPIVKPGPNCGFGKTGIIDYGIR LNRSERWDAYCYNPHAK | 23 |
| VPDF-1xTSG6 (Lava12) LC | AIYMHQEPSSLSASVGDRVTITCHGSYWLSNYLAWYQQKPGKAPKLLIYDGKEREHGVPSRF SGSGSHEDYTLTISSLQPEDFATYYCQQYRYHPYTFGHGTKVEIKRTVAAPSVFIFPPSDEQ LKSGTASVVCLLNNFYPREAKVQWKVDNALQSGNSQESVTEQDSKDSTYSLSSTLTLSKADY EKHKVYACEVTHQGLSSPVTKSFNRGEC | 24 |
| VPDF-2xCD44-knockout (ko) HC | DLQLVESGGGLVKPGGSLRLSCAADGWWFGYTDMSWVRQAPGKGLEWVGSISYKGGSTYYNT KFIGRFTISRDDDTNTLYLQMNSLRAEDTAVYYCARDDGYFDTWGQGTLVTVSSASTKGPSV FPLAPSSKSTSGGTAALGCLVKDYFPEPVTVSWNSGALTSGVHTFPAVLQSSGLYSLSSVVT VPSSSLGTQTYICNVNHKPSNTKVDKKVEPKSCGGGGSGGGGSGGGGSAQIDLNITCRFAGV FHVEKNGRSSISRTEAADLCKAFNSTLPTMAQMEKALSIGFETCRYGFIEGHVVIPRIHPNS ICAANNTGVYILTYNTSQYDTYCFNASAPPEEDCTSVTDLPNAFDGPITITIVNRDGTRYVQ KGEYRTNPEDIY | 25 |
| VPDF-2xCD44-knockout (ko) LC | AIYMHQEPSSLSASVGDRVTITCHGSYWLSNYLAWYQQKPGKAPKLLIYDGKEREHGVPSRF SGSGSHEDYTLTISSLQPEDFATYYCQQYRYHPYTFGHGTKVEIKRTVAAPSVFIFPPSDEQ LKSGTASVVCLLNNFYPREAKVQWKVDNALQSGNSQESVTEQDSKDSTYSLSSTLTLSKADY | 26 |
| | EKHKVYACEVTHQGLSSPVTKSFNRGECGGGGSGGGGSGGGGSAQIDLNITCRFAGVFHVEK NGRSSISRTEAADLCKAFNSTLPTMAQMEKALSIGFETCRYGFIEGHVVIPRIHPNSICAAN NTGVYILTYNTSQYDTYCFNASAPPEEDCTSVTDLPNAFDGPITITIVNRDGTRYVQKGEYR TNPEDIY | |
| Linker for RabFab-VG1, PigFab-VG1, G6.31.Fab-VG1, VPDF-VG1, VPDF-VG1ΔIg, 20D12v2.3-VG1 | GGGGS | 27 |
| Linker | GGGGSGGGGSGGGGS | 28 |
| WT VG1 | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 29 |
| VG Link1 | GSGVVFHYRA ATSRYTLNFE AAQKACLDVG AVIATPEQLF AAYEDGFEQC DAGWLADQTV RYPIRAPRVG CYGDKMGKAG VRTYGFRSPQ ETYDVYCYVD HHHHHHHH | 30 |
| VG Link2 | GDVFHLTVPS KFTFEEAAKE CENQDARLAT VGELQAAWRN GFDQCDYGWL SDASVRHPVT VARAQCGGGL LGVRTLYRFE NQTGFPPPDS RFDAYCFKPK EGNSHHHHHH HH | 31 |
| VG1ΔIg | VVFHYRAATS RYTLNFEAAQ KACLDVGAVI ATPEQLFAAY EDGFEQCDAG WLADQTVRYP IRAPRVGCYG DKMGKAGVRT YGFRSPQETY DVYCYVDHLD GDVFHLTVPS KFTFEEAAKE CENQDARLAT VGELQAAWRN GFDQCDYGWL SDASVRHPVT VARAQCGGGL LGVRTLYRFE NQTGFPPPDS RFDAYCFKPK EGNSHHHHHH HH | 32 |
| R160A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSA YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 33 |
| Y161A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR ATLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 34 |
| E194A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFAQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 35 |
| D197A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCAAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 36 |
| D197S | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCSAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 37 |
| Y208A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRAPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 38 |
| Y208F | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRFPI RAPRVGCYGD KMGKAGVRTY GFRSPQETYD VYCYVDHLDG DVFHLTVPSK FTFEEAAKEC | 39 |
| | ENQDARLATV GELQAAWRNG FDQCDYGWLS DASVRHPVTV ARAQCGGGLL GVRTLYRFEN QTGFPPPDSR FDAYCFKPKE GNSHHHHHHH H | |
| R214A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPAVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 40 |
| R214K | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPKVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 41 |
| M222A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KAGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 42 |
| Y230A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK | 43 |
| | ACLDVGAVIA TPEQLFAAYE DGFEQCDAGW LADQTVRYPI RAPRVGCYGD KMGKAGVRTA GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | |
| Y230F | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTF GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 44 |
| R233A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFASPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 45 |
| K260A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSA FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 46 |
| F261A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK ATFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 47 |
| D295A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCAYGWLS DASVRHPVTV ARAQCGGGLL GVRTLYRFEN QTGFPPPDSR FDAYCFKPKE GNSHHHHHHH H | 48 |
| Y296A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDAGWLS DASVRHPVTV ARAQCGGGLL GVRTLYRFEN QTGFPPPDSR FDAYCFKPKE GNSHHHHHHH H | 49 |
| H306A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC | 50 |
| | ENQDARLATV GELQAAWRNG FDQCDYGWLS DASVRAPVTV ARAQCGGGLL GVRTLYRFEN QTGFPPPDSR FDAYCFKPKE GNSHHHHHHH H | |
| R312A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV AAAQCGGGLL GVRTLYRFEN QTGFPPPDSR FDAYCFKPKE GNSHHHHHHH H | 51 |
| R312K | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV AKAQCGGGLL GVRTLYRFEN QTGFPPPDSR FDAYCFKPKE GNSHHHHHHH H | 52 |
| L325A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTAYRFEN QTGFPPPDSR FDAYCFKPKE GNSHHHHHHH H | 53 |
| Y326A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK | 54 |
| | ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLARFEN QTGFPPPDSR FDAYCFKPKE GNSHHHHHHH H | |
| R327A | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYAFEN QTGFPPPDSR FDAYCFKPKE GNSHHHHHHH H | 55 |
| RY160KF | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSK FTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 56 |
| LYR325LFK | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLFKFEN QTGFPPPDSR FDAYCFKPKE GNSHHHHHHH H | 57 |
| KF260RY | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSR YTFEEAAKEC ENQDARLATV GELQAAWRNG FDQCDYGWLS DASVRHPVTV ARAQCGGGLL GVRTLYRFEN QTGFPPPDSR FDAYCFKPKE GNSHHHHHHH H | 58 |
| DY295SF | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCSFGWLS DASVRHPVTV ARAQCGGGLL GVRTLYRFEN QTGFPPPDSR FDAYCFKPKE GNSHHHHHHH H | 59 |
| WT VG1 consensus sequence | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQC(D/S)AGW LADQTVRXPI RAP(R/K)VGCYGD KXGKAGVRTX GFRSPQETYD VYCYVDHLDG DVFHLTVPSX (F/Y)TFEEAAKEC ENQDARLATV GELQAAWRNG FDQC(D/S)XGWLS DASVRXPVTV AXAQCGGGLL GVRTXXXFEN QTGFPPPDSR FDAYCFKPKE | 60 |
| | X = any amino acid; (D/S) = Asp or Ser; (R/K) = Arg or Ser; amino acid in bold = wild type residue not mutated in some embodiments to enhance binding. | |
| RabFab, LC | ADVVMTQTPA SVSAAVGGTV TIKCQASQSI GTALAWYQQK PGQPPKLLIY RTSTLESGVP SRFKGSGSGT DFTLTISDLE CADAATYYCQ SAYVSGGNIY TFGGGTEVVV KGDPVAPTVL IFPPAADQVA TGTVTIVCVA NKYFPDVTVT WEVDGTTQTT GIENSKTPQN SADCTYNLSS TLTLTSTQYN GHKEYTCKVT QGTTSVVQSF NRGDC | 61 |
| RabFab, HC | QSLEESGGRL VTPGTPLTLT CTVSGFTISS YHMSWVRQAP GKGLEWIGIM RNTANIYYAS WAKGRFTISK TSPTTVDLKM TSLTTEDTAT YFCARGRPGD GALSLWGQGT LVTVSSGQPK APSVFPLAPC CGDTPSSTVT LGCLVKGYLP EPVTVTWNSG TLTNGVRTFP SVRQSSGLYS LSSVVSVTSS SQPVTCNVAH PATNTKVDKT VAPSTCSKPT | 62 |
| RabFab-VG1, LC | ADVVMTQTPA SVSAAVGGTV TIKCQASQSI GTALAWYQQK PGQPPKLLIY RTSTLESGVP SRFKGSGSGT DFTLTISDLE CADAATYYCQ SAYVSGGNIY TFGGGTEVVV KGDPVAPTVL IFPPAADQVA TGTVTIVCVA NKYFPDVTVT WEVDGTTQTT GIENSKTPQN SADCTYNLSS TLTLTSTQYN GHKEYTCKVT QGTTSVVQSF NRGDC | 63 |
| RabFab-VG1, HC | QSLEESGGRL VTPGTPLTLT CTVSGFTISS YHMSWVRQAP GKGLEWIGIM RNTANIYYAS WAKGRFTISK TSPTTVDLKM TSLTTEDTAT YFCARGRPGD GALSLWGQGT LVTVSSGQPK APSVFPLAPC CGDTPSSTVT LGCLVKGYLP EPVTVTWNSG TLTNGVRTFP SVRQSSGLYS LSSVVSVTSS SQPVTCNVAH PATNTKVDKT VAPSTCSKPT GGGGSLHKVK VGKSPPVRGS LSGKVSLPCH FSTMPTLPPS YNTSEFLRIK WSKIEVDKNG KDLKETTVLV AQNGNIKIGQ DYKGRVSVPT HPEAVGDASL TVVKLLASDA GLYRCDVMYG IEDTQDTVSL TVDGVVFHYR AATSRYTLNF EAAQKACLDV GAVIATPEQL FAAYEDGFEQ CDAGWLADQT VRYPIRAPRV GCYGDKMGKA GVRTYGFRSP QETYDVYCYV DHLDGDVFHL TVPSKFTFEE AAKECENQDA RLATVGELQA AWRNGFDQCD | 64 |
| | YGWLSDASVR HPVTVARAQC GGGLLGVRTL YRFENQTGFP PPDSRFDAYC FKPKEGNSHH HHHHHH | |
| PigFab-VG1, LC | AIQLTQSPAS LAASLGDTVS ITCRASQDVS TAVAWYQQQA GKAPKLLIYS ASFLYSGVPS RFKGSGSGTD FTLTISGLQA EDVATYYCQQ GYGNPFTFGQ GTKLELKRAD AKPSVFIFPP SKEQLETQTV SVVCLLNSFF PREVNVKWKV DGVVQSSGIL DSVTEQDSKD STYSLSSTLS LPTSQYLSHN LYSCEVTHKT LASPLVKSFS RNECEA | 65 |
| PigFab-VG1, HC | EEKLVESGGG LVQPGGSLRL SCVGSGFTIS DYWIHWVRQA PGKGLEWLAG ITPAGGYTYY ADSVKGRFTI SSDNSQNTAY LQMNSLRTED TARYYCARFV FFLPYAMDYW GPGVEVVVSS APKTAPSVYP LAPCSRDTSG PNVALGCLAS SYFPEPVTVT WNSGALSSGV HTFPSVLQPS GLYSLSSMVT VPASSLSSKS YTCNVNHPAT TTKVDKRVGT KTKGGGGSLH KVKVGKSPPV RGSLSGKVSL PCHFSTMPTL PPSYNTSEFL RIKWSKIEVD KNGKDLKETT VLVAQNGNIK IGQDYKGRVS VPTHPEAVGD ASLTVVKLLA SDAGLYRCDV MYGIEDTQDT VSLTVDGVVF HYRAATSRYT LNFEAAQKAC LDVGAVIATP EQLFAAYEDG FEQCDAGWLA DQTVRYPIRA PRVGCYGDKM GKAGVRTYGF RSPQETYDVY CYVDHLDGDV FHLTVPSKFT FEEAAKECEN QDARLATVGE LQAAWRNGFD QCDYGWLSDA SVRHPVTVAR AQCGGGLLGV RTLYRFENQT GFPPPDSRFD AYCFKPKE | 66 |
| G6.31.Fab-VG1, LC | DIQMTQSPSS LSASVGDRVT ITCRASQDVS TAVAWYQQKP GKAPKLLIYS ASFLYSGVPS RFSGSGSGTD FTLTISSLQP EDAATYYCQQ GYGAPFTFGQ GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC | 67 |
| G6.31.Fab-VG1 HC | EVQLVESGGG LVQPGGSLRL SCAASGFTIS DYWIHWVRQA PGKGLEWVAG ITPAGGYTRY ADSVKGRFTI SADTSKNTAY LQMRSLRAED TAVYYCARFV | 68 |
| | FFLPYAMDYW GQGTLVTVSS ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKVEP KSCDKTHTGG GGSLHKVKVG KSPPVRGSLS GKVSLPCHFS TMPTLPPSYN TSEFLRIKWS KIEVDKNGKD LKETTVLVAQ NGNIKIGQDY KGRVSVPTHP EAVGDASLTV VKLLASDAGL YRCDVMYGIE DTQDTVSLTV DGVVFHYRAA TSRYTLNFEA AQKACLDVGA VIATPEQLFA AYEDGFEQCD AGWLADQTVR YPIRAPRVGC YGDKMGKAGV RTYGFRSPQE TYDVYCYVDH LDGDVFHLTV PSKFTFEEAA KECENQDARL ATVGELQAAW RNGFDQCDYG WLSDASVRHP VTVARAQCGG GLLGVRTLYR FENQTGFPPP DSRFDAYCFK PKE | |
| VPDF-VG1, LC | AIYMHQEPSS LSASVGDRVT ITCHGSYWLS NYLAWYQQKP GKAPKLLIYD GKEREHGVPS RFSGSGSHED YTLTISSLQP EDFATYYCQQ YRYHPYTFGH GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC | 69 |
| VPDF-VG1, HC | DLQLVESGGG LVKPGGSLRL SCAADGWWFG YTDMSWVRQA PGKGLEWVGS ISYKGGSTYY NTKFIGRFTI SRDDDTNTLY LQMNSLRAED TAVYYCARDD GYFDTWGQGT LVTVSSASTK GPSVFPLAPS SKSTSGGTAA LGCLVKDYFP EPVTVSWNSG ALTSGVHTFP AVLQSSGLYS LSSVVTVPSS SLGTQTYICN VNHKPSNTKV DKKVEPKSCD KTHTGGGGSL HKVKVGKSPP VRGSLSGKVS LPCHFSTMPT LPPSYNTSEF LRIKWSKIEV DKNGKDLKET TVLVAQNGNI KIGQDYKGRV SVPTHPEAVG DASLTVVKLL ASDAGLYRCD VMYGIEDTQD TVSLTVDGVV FHYRAATSRY TLNFEAAQKA CLDVGAVIAT PEQLFAAYED GFEQCDAGWL ADQTVRYPIR APRVGCYGDK MGKAGVRTYG FRSPQETYDV YCYVDHLDGD VFHLTVPSKF TFEEAAKECE NQDARLATVG ELQAAWRNGF | 70 |
| | DQCDYGWLSD ASVRHPVTVA RAQCGGGLLG VRTLYRFENQ TGFPPPDSRF DAYCFKPKE | |
| VG1-Fc (2x) | LHKVKVGKSP PVRGSLSGKV SLPCHFSTMP TLPPSYNTSE FLRIKWSKIE VDKNGKDLKE TTVLVAQNGN IKIGQDYKGR VSVPTHPEAV GDASLTVVKL LASDAGLYRC DVMYGIEDTQ DTVSLTVDGV VFHYRAATSR YTLNFEAAQK ACLDVGAVIA TPEQLFAAYE DGFEQCDAGW LADQTVRYPI RAPRVGCYGD KMGKAGVRTY GFRSPQETYD VYCYVDHLDG DVFHLTVPSK FTFEEAAKEC ENQDARLATV GELQAAWRNG FDQCDYGWLS DASVRHPVTV ARAQCGGGLL GVRTLYRFEN QTGFPPPDSR FDAYCFKRKC LIPFGNSVTD KTHTCPPCPA PELLGGPSVF LFPPKPKDTL MISRTPEVTC VVVDVSHEDP EVKFNWYVDG VEVHNAKTKP REEQYNSTYR VVSVLTVLHQ DWLNGKEYKC KVSNKALPAP IEKTISKAKG QPREPQVYTL PPSREEMTKN QVSLTCLVKG FYPSDIAVEW ESNGQPENNY KTTPPVLDSD GSFFLYSKLT VDKSRWQQGN VFSCSVMHEA LHNHYTQKSL SLSPGK | 71 |
| VPDF-VG1ΔIg, LC | AIYMHQEPSS LSASVGDRVT ITCHGSYWLS NYLAWYQQKP GKAPKLLIYD GKEREHGVPS RFSGSGSHED YTLTISSLQP EDFATYYCQQ YRYHPYTFGH GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC | 72 |
| VPDF-VG1ΔIg, HC | DLQLVESGGG LVKPGGSLRL SCAADGWWFG YTDMSWVRQA PGKGLEWVGS ISYKGGSTYY NTKFIGRFTI SRDDDTNTLY LQMNSLRAED TAVYYCARDD GYFDTWGQGT LVTVSSASTK GPSVFPLAPS SKSTSGGTAA LGCLVKDYFP EPVTVSWNSG ALTSGVHTFP AVLQSSGLYS LSSVVTVPSS SLGTQTYICN VNHKPSNTKV DKKVEPKSCD KTHTGGGGSG VVFHYRAATS RYTLNFEAAQ KACLDVGAVI ATPEQLFAAY EDGFEQCDAG WLADQTVRYP IRAPRVGCYG DKMGKAGVRT YGFRSPQETY DVYCYVDHLD GDVFHLTVPS KFTFEEAAKE | 73 |
| | CENQDARLAT VGELQAAWRN GFDQCDYGWL SDASVRHPVT VARAQCGGGL LGVRTLYRF ENQTGFPPPD SRFDAYCFKP KE | |
| 20D12v2.3-VG1, LC | DIQMTQSPSS LSASVGDRVT ITCKASQNVD TDVAWFQQKP GKAPKGLIRS ASSRYSGVPS RFSGSGSGTD FTLTISSLQP EDFATYYCQQ YNNYPLTFGQ GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC | 74 |
| 20D12v2.3-VG1, HC | EVQLVQSGAE VKKPGASVKV SCKASGYTFT SYYMYWVRQA PGQGLEWIGE INPTSGGTNF NEKFKSRATL TVDTSTSTAY LELSSLRSED TAVYYCAREG GFAYWGQGTL VTVSSASTKG PSVFPLAPSS KSTSGGTAAL GCLVKDYFPE PVTVSWNSGA LTSGVHTFPA VLQSSGLYSL SSVVTVPSSS LGTQTYICNV NHKPSNTKVD KKVEPKSCDK THTGGGGSLH KVKVGKSPPV RGSLSGKVSL PCHFSTMPTL PPSYNTSEFL RIKWSKIEVD KNGKDLKETT VLVAQNGNIK IGQDYKGRVS VPTHPEAVGD ASLTVVKLLA SDAGLYRCDV MYGIEDTQDT VSLTVDGVVF HYRAATSRYT LNFEAAQKAC LDVGAVIATP EQLFAAYEDG FEQCDAGWLA DQTVRYPIRA PRVGCYGDKM GKAGVRTYGF RSPQETYDVY CYVDHLDGDV FHLTVPSKFT FEEAAKECEN QDARLATVGE LQAAWRNGFD QCDYGWLSDA SVRHPVTVAR AQCGGGLLGV RTLYRFENQT GFPPPDSRFD AYCFKPKE | 75 |
| Ranibizumab-VG1, LC | DIQLTQSPSS LSASVGDRVT ITCSASQDIS NYLNWYQQKP GKAPKVLIYF TSSLHSGVPS RFSGSGSGTD FTLTISSLQP EDFATYYCQQ YSTVPWTFGQ GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC | 76 |
| Ranibizumab-VG1, HC | EVQLVESGGG LVQPGGSLRL SCAASGYDFT HYGMNWVRQA PGKGLEWVGW INTYTGEPTY AADFKRRFTF SLDTSKSTAY LQMNSLRAED TAVYYCAKYP YYYGTSHWYF DVWGQGTLVT VSSASTKGPS VFPLAPSSKS TSGGTAALGC LVKDYFPEPV TVSWNSGALT SGVHTFPAVL QSSGLYSLSS VVTVPSSSLG TQTYICNVNH KPSNTKVDKK VEPKSCDKTH TGGGGLHKVK VGKSPPVRGS LSGKVSLPCH FSTMPTLPPS YNTSEFLRIK WSKIEVDKNG KDLKETTVLV AQNGNIKIGQ DYKGRVSVPT HPEAVGDASL TVVKLLASDA GLYRCDVMYG IEDTQDTVSL TVDGVVFHYR AATSRYTLNF EAAQKACLDV GAVIATPEQL FAAYEDGFEQ CDAGWLADQT VRYPIRAPRV GCYGDKMGKA GVRTYGFRSP QETYDVYCYV DHLDGDVFHL TVPSKFTFEE AAKECENQDA RLATVGELQA AWRNGFDQCD YGWLSDASVR HPVTVARAQC GGGLLGVRTL YRFENQTGFP PPDSRFDAYC FKPKE | 77 |
| EETI-VG1 | GCPRILMRCK QDSDCLAGCV CGPNGFCGGS GSGSGSGSLH KVKVGKSPPV RGSLSGKVSL PCHFSTMPTL PPSYNTSEFL RIKWSKIEVD KNGKDLKETT VLVAQNGNIK IGQDYKGRVS VPTHPEAVGD ASLTVVKLLA SDAGLYRCDV MYGIEDTQDT VSLTVDGVVF HYRAATSRYT LNFEAAQKAC LDVGAVIATP EQLFAAYEDG FEQCDAGWLA DQTVRYPIRA PRVGCYGDKM GKAGVRTYGF RSPQETYDVY CYVDHLDGDV FHLTVPSKFT FEEAAKECEN QDARLATVGE LQAAWRNGFD QCDYGWLSDA SVRHPVTVAR AQCGGGLLGV RTLYRFENQT GFPPPDSRFD AYCFKPKEGN SHHHHHHHH | 78 |
| EETI-TEV-VG1 | GCPRILMRCK QDSDCLAGCV CGPNGFCGEN LYFQGSGSGS GSGSLHKVKV GKSPPVRGSL SGKVSLPCHF STMPTLPPSY NTSEFLRIKW SKIEVDKNGK DLKETTVLVA QNGNIKIGQD YKGRVSVPTH PEAVGDASLT VVKLLASDAG LYRCDVMYGI EDTQDTVSLT VDGVVFHYRA ATSRYTLNFE AAQKACLDVG AVIATPEQLF AAYEDGFEQC DAGWLADQTV RYPIRAPRVG CYGDKMGKAG VRTYGFRSPQ ETYDVYCYVD HLDGDVFHLT VPSKFTFEEA AKECENQDAR | 79 |
| | LATVGELQAA WRNGFDQCDY GWLSDASVRH PVTVARAQCG GGLLGVRTLY RFENQTGFPP PDSRFDAYCF KPKEGNSHHH HHHHH | |
| VG1-EETI | MGGTAARLGA VILFVVIVGL HGVRHHHHHH HHGENLYFQG SLHKVKVGKS PPVRGSLSGK VSLPCHFSTM PTLPPSYNTS EFLRIKWSKI EVDKNGKDLK ETTVLVAQNG NIKIGQDYKG RVSVPTHPEA VGDASLTVVK LLASDAGLYR CDVMYGIEDT QDTVSLTVDG VVFHYRAATS RYTLNFEAAQ KACLDVGAVI ATPEQLFAAY EDGFEQCDAG WLADQTVRYP IRAPRVGCYG DKMGKAGVRT YGFRSPQETY DVYCYVDHLD GDVFHLTVPS KFTFEEAAKE CENQDARLAT VGELQAAWRN GFDQCDYGWL SDASVRHPVT VARAQCGGGL LGVRTLYRFE NQTGFPPPDS RFDAYCFKPK EGNSGSGSGS GSGSGCPRIL MRCKQDSDCL AGCVCGPNGF CG | 80 |
| VG1-TEV-EETI | MGGTAARLGA VILFVVIVGL HGVRHHHHHH HHGENLYFQG SLHKVKVGKS PPVRGSLSGK VSLPCHFSTM PTLPPSYNTS EFLRIKWSKI EVDKNGKDLK ETTVLVAQNG NIKIGQDYKG RVSVPTHPEA VGDASLTVVK LLASDAGLYR CDVMYGIEDT QDTVSLTVDG VVFHYRAATS RYTLNFEAAQ KACLDVGAVI ATPEQLFAAY EDGFEQCDAG WLADQTVRYP IRAPRVGCYG DKMGKAGVRT YGFRSPQETY DVYCYVDHLD GDVFHLTVPS KFTFEEAAKE CENQDARLAT VGELQAAWRN GFDQCDYGWL SDASVRHPVT VARAQCGGGL LGVRTLYRFE NQTGFPPPDS RFDAYCFKPK EGNSGSGSGS GSGSENLYFQ GGCPRILMRC KQDSDCLAGC VCGPNGFCG | 81 |
| Linker for VG1-Fc (2x) | RKCLIPFGNSVT | 82 |
| Linker for Ranibizumab-VG1 | GGGG | 83 |
| Linker for EETI-VG1, VG1-EETI | GSGSGSGSGS | 84 |
| Linker for EETI-TEV-VG1, VG1-TEV-EETI | ENLYFQGSGSGSGSGS | 85 |
| VG1ΔIg consensus sequence | VVFHYRAATS RYTLNFEAAQ KACLDVGAVI ATPEQLFAAY EDGFEQC(D/S)AG WLADQTVRXP IRAP(R/K)VGCYG DKXGKAGVRT XGFRSPQETY DVYCYVDHLD GDVFHLTVPS X(F/Y)TFEEAAKE CENQDARLAT VGELQAAWRN GFDQC(D/S)XGWL SDASVRXPVT VAXAQCGGGL LGVRTXXXFE NQTGFPPPDS RFDAYCFKPK E | 86 |
| | X = any amino acid; (D/S) = Asp or Ser; (R/K) = Arg or Ser; amino acid in bold = wild type residue not mutated in some embodiments to enhance binding. | |
| Linker | (GGGS) 3-6 | 87 |
| Linker | (GGGGS) 2-5 | 88 |
| Linker | (GGGS) 3-6 (G) 0-3 | 89 |
| Linker | (GGGGS) 2-5 (G) 0-3 | 90 |
| Linker | GGGS | 91 |
| Anti-VEGF cysteine knot peptide (CKP) for VG1 fusion modified L3.54.90.67.F8Y. M5 VC072M | GCNIMLPYWGCGRDFECMEQCICQYYQSCG | 92 |
| Fusion 5; VC072M.GS10X. VG1CTH | GCNIMLPYWGCGRDFECMEQCICQYYQSCG.GS10X.VG1CTH GS10X = (GS)₁₀; VG1CTH = SEQ ID NO: 29 | 93 |
| Fusion 6; VG1NTH.GS10X. VC072M | VG1NTH.GS10X.GCNIMLPYWGCGRDFECMEQCICQYYQSCG GS10X = (GS)₁₀; VG1NTH = VG1 with N-terminal his-tag | 94 |
| Linker used in SEQ ID NOs: 93 and 94 | GSGSGSGSGSGSGSGSGSGS | 95 |
| CD44-ko domain | AQIDLNITCR FAGVFHVEKN GRSSISRTEA ADLCKAFNST LPTMAQMEKA LSIGFETCRY GFIEGHVVIP RIHPNSICAA NNTGVYILTY NTSQYDTYCF NASAPPEEDC TSVTDLPNAF DGPITITIVN RDGTRYVQKG EYRTNPEDIY | 96 |
| PigFab VH | EEKLVESGGG LVQPGGSLRL SCVGSGFTIS DYWIHWVRQA PGKGLEWLAG ITPAGGYTYY ADSVKGRFTI SSDNSQNTAY LQMNSLRTED TARYYCARFV FFLPYAMDYW GPGVEVVVSS | 97 |
| PigFab VL | AIQLTQSPAS LAASLGDTVS ITCRASQDVS TAVAWYQQQA GKAPKLLIYS ASFLYSGVPS RFKGSGSGTD FTLTISGLQA EDVATYYCQQ GYGNPFTFGQ GTKLELK | 98 |
| VPDF VH | DLQLVESGGG LVKPGGSLRL SCAADGWWFG YTDMSWVRQA PGKGLEWVGS ISYKGGSTYY NTKFIGRFTI SRDDDTNTLY LQMNSLRAED TAVYYCARDD GYFDTWGQGT LVTVSS | 99 |
| VPDF VL | AIYMHQEPSS LSASVGDRVT ITCHGSYWLS NYLAWYQQKP GKAPKLLIYD GKEREHGVPS RFSGSGSHED YTLTISSLQP EDFATYYCQQ YRYHPYTFGH GTKVEIK | 100 |
| VPDF (unmodified) HC | DLQLVESGGG LVKPGGSLRL SCAADGWWFG YTDMSWVRQA PGKGLEWVGS ISYKGGSTYY NTKFIGRFTI SRDDDTNTLY LQMNSLRAED TAVYYCARDD GYFDTWGQGT LVTVSSASTK GPSVFPLAPS SKSTSGGTAA LGCLVKDYFP EPVTVSWNSG ALTSGVHTFP AVLQSSGLYS LSSVVTVPSS SLGTQTYICN VNHKPSNTKV DKKVEPKSCD KTHT | 101 |
| VPDF (unmodified) LC | AIYMHQEPSS LSASVGDRVT ITCHGSYWLS NYLAWYQQKP GKAPKLLIYD GKEREHGVPS RFSGSGSHED YTLTISSLQP EDFATYYCQQ YRYHPYTFGH | 102 |
| | GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC | |
| G6.31 Fab (unmodified) HC | EVQLVESGGG LVQPGGSLRL SCAASGFTIS DYWIHWVRQA PGKGLEWVAG ITPAGGYTRY ADSVKGRFTI SADTSKNTAY LQMRSLRAED TAVYYCARFV FFLPYAMDYW GQGTLVTVSS ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKVEP KSCDKTHT | 103 |
| G6.31 Fab (unmodified) LC | DIQMTQSPSS LSASVGDRVT ITCRASQDVS TAVAWYQQKP GKAPKLLIYS ASFLYSGVPS RFSGSGSGTD FTLTISSLQP EDAATYYCQQ GYGAPFTFGQ GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC | 104 |
| G6.31 VH | EVQLVESGGG LVQPGGSLRL SCAASGFTIS DYWIHWVRQA PGKGLEWVAG ITPAGGYTRY ADSVKGRFTI SADTSKNTAY LQMRSLRAED TAVYYCARFV FFLPYAMDYW GQGTLVTVSS | 105 |
| G6.31 VL | DIQMTQSPSS LSASVGDRVT ITCRASQDVS TAVAWYQQKP GKAPKLLIYS ASFLYSGVPS RFSGSGSGTD FTLTISSLQP EDAATYYCQQ GYGAPFTFGQ GTKVEIK | 106 |
| RabFab VH | QSLEESGGRL VTPGTPLTLT CTVSGFTISS YHMSWVRQAP GKGLEWIGIM RNTANIYYAS WAKGRFTISK TSPTTVDLKM TSLTTEDTAT YFCARGRPGD GALSLWGQGT LVTVSS | 107 |
| RabFab VL | ADVVMTQTPA SVSAAVGGTV TIKCQASQSI GTALAWYQQK PGQPPKLLIY RTSTLESGVP SRFKGSGSGT DFTLTISDLE CADAATYYCQ SAYVSGGNIY TFGGGTEVVV K | 108 |
| NVS24 VH | EVQLVESGGG LVQPGGSLRL SCTASGFSLT NYYYMTWVRQ APGKGLEWVG FIDPQNDPYY ATWAKGRFTI SRDNSKNTLY LQMNSLRAED TAVYYCAGGN HNSGWGLNIW GQGTLVTVSS | 109 |
| NVS24 VL | EIVMTQSPST LSASVGDRVI ITCQASQKIH SWLAWYQQKP GKAPKLLIYQ ASKLAKGVPS RFSGSGSGAE FTLTISSLQP DDFATYYCQN VYLASTNGAN FGQGTKLTVL | 110 |
| 20D12v2.3 VH | EVQLVQSGAE VKKPGASVKV SCKASGYTFT SYYMYWVRQA PGQGLEWIGE INPTSGGTNF NEKFKSRATL TVDTSTSTAY LELSSLRSED TAVYYCAREG GFAYWGQGTL VTVSS | 111 |
| 20D12v2.3 VL | DIQMTQSPSS LSASVGDRVT ITCKASQNVD TDVAWFQQKP GKAPKGLIRS ASSRYSGVPS RFSGSGSGTD FTLTISSLQP EDFATYYCQQ YNNYPLTFGQ GTKVEIK | 112 |
| TSG6 (Lava12) | GVYHREAISG KYYLTYAEAK AVCEFEGGHL ATYKQLLAAQ KIGFHVCAAG WMAKGRVGYP IVKPGPNCGF GKTGIIDYGI RLNRSERWDA YCYNPHA | 113 |
| ranibizumab VH | EVQLVESGGG LVQPGGSLRL SCAASGYDFT HYGMNWVRQA PGKGLEWVGW INTYTGEPTY AADFKRRFTF SLDTSKSTAY LQMNSLRAED TAVYYCAKYP YYYGTSHWYF DVWGQGTLVT VSS | 114 |
| ranibizumab VL | DIQLTQSPSS LSASVGDRVT ITCSASQDIS NYLNWYQQKP GKAPKVLIYF TSSLHSGVPS RFSGSGSGTD FTLTISSLQP EDFATYYCQQ YSTVPWTFGQ GTKVEIK | 115 |
| Anti-HtrA1 VH | EVQLVQSGAE VKKPGASVKV SCKASGYKFT DSEMHWVRQA PGQGLEWIGG VDPETEGAAY NQKFKGRATI TRDTSTSTAY LELSSLRSED TAVYYCTRGY DYDYALDYWG QGTLVTVSS | 116 |
| Anti-HtrA1 VL | DIQMTQSPSS LSASVGDRVT ITCRASSSVE FIHWYQQKPG KAPKPLISAT SNLASGVPSR FSGSGSGTDF TLTISSLQPE DFATYYCQQW SSAPWTFGQG TKVEIK | 117 |
| Anti-HtrA1-VG1 HC | EVQLVQSGAEVKKPGASVKVSCKASGYKFTDSEMHWVRQAPGQGLEWIGGVDPETEGAAYNQ KFKGRATITRDTSTSTAYLELSSLRSEDTAVYYCTRGYDYDYALDYWGQGTLVTVSSASTKG | 118 |
| | PSVFPLAPSSKSTSGGTAALGCLVKDYFPEPVTVSWNSGALTSGVHTFPAVLQSSGLYSLSS VVTVPSSSLGTQTYICNVNHKPSNTKVDKKVEPKSCDKTHTGGGGLHKVKVGKSPPVRGSLS GKVSLPCHFSTMPTLPPSYNTSEFLRIKWSKIEVDKNGKDLKETTVLVAQNGNIKIGQDYKG RVSVPTHPEAVGDASLTVVKLLASDAGLYRCDVMYGIEDTQDTVSLTVDGVVFHYRAATSRY TLNFEAAQKACLDVGAVIATPEQLFAAYEDGFEQCDAGWLADQTVRYPIRAPRVGCYGDKMG KAGVRTYGFRSPQETYDVYCYVDHLDGDVFHLTVPSKFTFEEAAKECENQDARLATVGELQA AWRNGFDQCDYGWLSDASVRHPVTVARAQCGGGLLGVRTLYRFENQTGFPPPDSRFDAYCFK PKE | |
| Anti-HtrA1-VG1 LC | DIQMTQSPSSLSASVGDRVTITCRASSSVEFIHWYQQKPGKAPKPLISATSNLASGVPSRFS GSGSGTDFTLTISSLQPEDFATYYCQQWSSAPWTFGQGTKVEIKRTVAAPSVFIFPPSDEQL KSGTASVVCLLNNFYPREAKVQWKVDNALQSGNSQESVTEQDSKDSTYSLSSTLTLSKADYE KHKVYACEVTHQGLSSPVTKSFNRGEC | 119 |
| Anti-gD Fab HC | EVQLVESGGGLVQPGGSLRLSCAASGYSITSDFAWNWVRQAPGKGLEWVGYISYSGTTSYNP SLKSRITISRDNSKNTFYLQMNSLRAEDTAVYYCARENYYGRSHVGYFDVWGQGTLVTVSSA STKGPSVFPLAPSSKSTSGGTAALGCLVKDYFPEPVTVSWNSGALTSGVHTFPAVLQSSGLY SLSSVVTVPSSSLGTQTYICNVNHKPSNTKVDKKVEPKSCDKTHT | 120 |
| Anti-gD Fab LC (also referred to herein as Anti-gD IgG LC and Anti-gD Fab-VG1 LC of BRD) | DIQMTQSPSSLSASVGDRVTITCRASASVDSYGNSFIHWYQQKPGKAPKLLIYRASDLESGV PSRFSGSGSGTDFTLTISSLQPEDFATYYCQQNYADPFTFGQGTKVEIKRTVAAPSVFIFPP SDEQLKSGTASVVCLLNNFYPREAKVQWKVDNALQSGNSQESVTEQDSKDSTYSLSSTLTLS KADYEKHKVYACEVTHQGLSSPVTKSFNRGEC | 121 |
| Anti-gD IgG HC | EVQLVESGGGLVQPGGSLRLSCAASGYSITSDFAWNWVRQAPGKGLEWVGYISYSGTTSYNP SLKSRITISRDNSKNTFYLQMNSLRAEDTAVYYCARENYYGRSHVGYFDVWGQGTLVTVSSA STKGPSVFPLAPSSKSTSGGTAALGCLVKDYFPEPVTVSWNSGALTSGVHTFPAVLQSSGLY SLSSVVTVPSSSLGTQTYICNVNHKPSNTKVDKKVEPKSCDKTHTCPPCPAPELLGGPSVFL | 122 |
| | FPPKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAKGQPREPQVYTLPPSREEMTKNQVSLT CLVKGFYPSDIAVEWESNGQPENNYKTTPPVLDSDGSFFLYSKLTVDK SRWQQGNVFSCSVMHEALHNHYTQKSLSLSPGK | |
| Anti-gD Fab-VG1 HC of BRD | EVQLVESGGGLVQPGGSLRLSCAASGYSITSDFAWNWVRQAPGKGLEWVGYISYSGTTSYNP SLKSRITISRDNSKNTFYLQMNSLRAEDTAVYYCARENYYGRSHVGYFDVWGQGTLVTVSSA STKGPSVFPLAPSSKSTSGGTAALGCLVKDYFPEPVTVSWNSGALTSGVHTFPAVLQSSGLY SLSSVVTVPSSSLGTQTYICNVNHKPSNTKVDKKVEPKSCDKTHTGGGGSLHKVKVGKSPPV RGSLSGKVSLPCHFSTMPTLPPSYNTSEFLRIKWSKIEVDKNGKDLKETTVLVAQNGNIKIG QDYKGRVSVPTHPEAVGDASLTVVKLLASDAGLYRCDVMYGIEDTQDTVSLTVDGVVFHYRA ATSRYTLNFEAAQKACLDVGAVIATPEQLFAAYEDGFEQCDAGWLADQTVRYPIRAPRVGCY GDKMGKAGVRTYGFRSPQETYDVYCYVDHLDGDVFHLTVPSKFTFE EAAKECENQDARLATVGELQAAWRNGFDQCDYGWLSDASVRHPVTVARAQCGGGLLGVRT LYRFENQTGFPPPDSRFDAYCFKPKE | 124 |

### DETAILED DESCRIPTION

### I. Definitions

Unless defined otherwise, all technical and scientific terms and any acronyms used herein have the same meanings as commonly understood by one of ordinary skill in the art in the field of the disclosure. Although any methods and materials similar or equivalent to those described herein can be used in the practice as presented herein, the specific methods, and materials are described herein.

Unless stated otherwise, the following terms and phrases as used herein are intended to have the following meanings:

The term “antibody” as used herein means a full (complete or intact) antibody. An antibody is a glycoprotein comprising at least two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (CIq) of the classical complement system.

The term “antigen-binding fragment” of an antibody or “antibody fragment”, as used herein, refers to one or more fragments of an antibody that retain the ability to specifically bind to a given antigen (e.g., the therapeutic target in the eye, such as VEGF) and thus exhibit the desired antigen-binding activity. Antigen-binding functions of an antibody can be performed by fragments of an intact antibody. Examples of binding fragments encompassed within the term antigen-binding fragment of an antibody include, but are not limited to Examples of antibody fragments include but are not limited to Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments; an Fd fragment consisting of the VH and CH1 domains; an Fv fragment consisting of the VL and VH domains of a single arm of an antibody; a single domain antibody (dAb) fragment, which consists of a VH domain or a VL domain; and an isolated complementarity determining region (CDR). For a review of certain antibody fragments, see Holliger and Hudson, Nature Biotechnology 23:1126-1136 (2005). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv). Such single chain antibodies may include one or more antigen-binding fragments of an antibody. These antigen-binding fragments are obtained using conventional techniques known to those of skill in the art, and the fragments are screened for utility in the same manner as are intact antibodies. Antigen-binding fragments can also be incorporated into single domain antibodies, maxibodies, minibodies, intrabodies, diabodies, triabodies, tetrabodies, v-NAR and bis-scFv). Antigen-binding fragments can be incorporated into single chain molecules comprising a pair of tandem Fv segments (VH-CH1-VH-CH1) which, together with complementary light chain polypeptides, form a pair of antigen-binding regions. The term “antibodies” includes polyclonal antibodies and monoclonal antibodies.

Aptamers are oligonucleotide or peptide molecules that bind to a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications. More specifically, aptamers can be classified as DNA or RNA or XNA aptamers, which consist of (usually short) strands of oligonucleotides, and peptide aptamers, which consist of one (or more) short variable peptide domains, attached at both ends to a protein scaffold. Both DNA and RNA aptamers show robust binding affinities for various targets. DNA and RNA aptamers have been selected for the same target. These targets include lysozyme, thrombin, interferon γ, vascular endothelial growth factor (VEGF), dopamine. In the case of e.g., VEGF the DNA aptamer is the analog of the RNA aptamer, with thymine replacing uracil.

A “covalent bond,” also called a molecular bond, is a chemical bond that involves the sharing of electron pairs between atoms. These electron pairs are known as shared pairs or bonding pairs, and the stable balance of attractive and repulsive forces between atoms, when they share electrons, is known as covalent bonding.

As used herein, the term “DARPin” (an acronym for designed ankyrin repeat proteins) refers to an antibody mimetic protein typically exhibiting highly specific and high-affinity target protein binding. They are typically genetically engineered and derived from natural ankyrin proteins and consist of at least three, usually four or five repeat motifs of these proteins. Their molecular mass is about 14 or 18 kDa for four- or five-repeat DARPins, respectively. Examples of DARPins can be found, for example in U.S. Pat. 7,417,130.

An “effective amount” of an agent, e.g., a pharmaceutical composition, refers to an amount effective, at dosages and for periods of time necessary, to achieve the desired therapeutic or prophylactic result.

The term “eye disease” as used herein, includes any eye disease associated with pathological angiogenesis and/or atrophy. The term “eye disease” is synonymous with the terms “eye condition,” “eye disorder,” “ocular condition,” “ocular disease,” and “ocular disorder.”

As used herein, “Fab-hyaluronan-binding domain,” “Fab-hyaluronic acid binding domain,” and “Fab-HABD” refer to a fusion protein that comprises a Fab and a hyaluronan-binding domain. These terms are synonymous and may be used interchangeably throughout the current disclosure.

As used herein, “hyaluronan,” “hyaluronic acid,” “hyaluronate,” and “HA” refer to a non-sulfated glucosaminoglycan with the chemical formula (C₁₄H₂₁NO₁₁)ₙ and salts thereof.

As used herein, “hyaluronic acid binding domain,” “hyaluronic acid binding moiety,” “HA binding domain” or “HABD” refers to any moiety that is capable of binding hyaluronic acid. In some instances, the HABD may be a domain of a HA-binding protein.

A ligand is a substance that forms a complex or a conjugate with a biomolecule to serve a biological purpose. In protein-ligand binding, the ligand is usually a molecule which produces a signal by binding to a site on a target protein. The binding typically results in a change of conformational isomerism (conformation) of the target protein. In DNA-ligand binding studies, the ligand can be a small molecule, ion, or protein which binds to the DNA double helix. The relationship between ligand and binding partner is a function of charge, hydrophobicity, and molecular structure. The instance of binding occurs over an infinitesimal range of time and space, so the rate constant is usually a very small number. The ligand may be a naturally occurring ligand or a non-naturally occurring ligand. Additionally, it may agonist, partial agonist, antagonist, or inverse agonist.

A “non-covalent interaction” differs from a covalent bond in that it does not involve the sharing of electrons, but rather involves more dispersed variations of electromagnetic interactions between molecules or within a molecule. Non-covalent interactions can be classified into different categories, such as electrostatic, π-effects, van der Waals forces, and hydrophobic effects. Preferably, the conjugate is provided in isolated form. The first and second component may be covalently bound to each other via a linker or directly.

Nucleic acids are the biopolymers composed of nucleotides, which are the monomers made of three components: a 5-carbon sugar, a phosphate group and a nitrogenous base. The term nucleic acid is the overall name for DNA and RNA. If the sugar is a compound ribose, the polymer is RNA (ribonucleic acid); if the sugar is derived from ribose as deoxyribose, the polymer is DNA (deoxyribonucleic acid).

The term “peptide linker” as used herein denotes a peptide with amino acid sequences, which is preferably of synthetic origin.

Proteins are large biopolymers (polypeptides) consisting of one or more long chains of amino acid residues. Proteins perform a vast array of functions within organisms, including catalyzing metabolic reactions, DNA replication, responding to stimuli, providing structure to cells and organisms, and transporting molecules from one location to another. Proteins differ from one another primarily in their sequence of amino acids, which is dictated by the nucleotide sequence of their genes, and which usually results in protein folding into a specific three-dimensional structure that determines its activity. Short polypeptides, containing less than 20-30 residues, are commonly called peptides.

As used herein, a “protein conjugate” or “conjugate” refers to a protein that is non-covalently bound to hyaluronan.

Receptors are chemical structures, usually composed of proteins, that receive and transduce signals that may be integrated into biological systems. These signals are typically chemical messengers (ligands), which bind to a receptor, they cause some form of cellular/tissue response, e.g., a change in the activity of a cell. There are three main ways the action of the receptor can be classified: relay of signal, amplification, or integration. Relaying sends the signal onward, amplification increases the effect of a single ligand, and integration allows the signal to be incorporated into another biochemical pathway. In this sense, a receptor is a protein-molecule that recognizes and responds to endogenous chemical signals. Therefore, receptor or fragments comprising the ligand-binding site and their ligands are suitable binding counterparts (first component and therapeutic target) in the context of the invention.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

All numbers in the specification and claims are modified by the term “about”. This means that each number includes minor variations as defined as 10% of the numerical value or range in questions.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

Reference will now be made in detail to certain embodiments of the invention, examples of which are illustrated in the accompanying figures, examples, and embodiments. It will be understood that the figures, examples, and embodiments (unless otherwise specifically indicated) are not intended to limit the scope of the invention to particular methodology, protocols, and reagents described herein because they may vary. The invention is intended to cover all alternatives, modifications, and equivalents, which may be included within the invention as defined by the appended claims and included embodiments. Further, the terminology used herein is for the purpose of describing particular embodiments only and is not intended to limit the scope of the present disclosure. As used herein and in the appended claims, the singular forms “a”, “an”, and “the” include plural reference unless the context clearly dictates otherwise. Similarly, the words “comprise,” “contain,” and “encompass” are to be interpreted inclusively rather than exclusively.

The section headings used herein are for organizational purposes only and are not to be construed as limiting the desired subject matter in any way. All publications (scientific and patent publications) cited herein are incorporated by reference. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### II. A Therapeutic Molecule Comprising a Therapeutically Active Agent (i.e., a First Component) and a Hyaluronan-Binding Domain (HABD; i.e., a Second Component)

The present application provides therapeutic molecules targeted to a tissue in a patient comprising a therapeutically active agent and an HA-binding domain (HABD). Each therapeutic molecule comprises a first component and one or more second components. The first component is capable of binding to a therapeutic target in the eye. The second components are capable of binding to HA and therefore comprises a HA binding domain (HABD).

In some embodiments, the therapeutic molecule is a fusion protein with a first component and one or more second components. The first and second components are covalently bound to each other thereby forming a fusion protein. In some embodiments, the therapeutic molecule further comprises a peptide linker.

In some embodiments, the therapeutic molecule comprises one second component. In some embodiments, the therapeutic molecule comprises two or more second components. Particularly, if an antibody or antigen-binding fragment thereof is used, which is composed of two proteins (i.e., one heavy chain or fragment thereof, and one light chain or fragment thereof), the therapeutic molecule may comprise two second components. In these embodiments, a first second component is linked to the heavy chain of the antibody or antigen-binding fragment and a second second component is linked to the light chain of the antibody or antigen-binding fragment. In some embodiments, the first second component is linked to the C-terminus of the heavy chain of an Fab fragment and the second second component is linked to the C-terminus of the light chain of an Fab fragment.

In some embodiments, the therapeutic molecule further comprises (in addition to the first and second components) one or more third components. The second components are covalently bound to the first component, and the second components are non-covalently bound to the third components. In some embodiments, the third component is hyaluronan (HA). In some of these embodiments, the second components are capable of binding HA and the therapeutic molecule protein (i.e., the first component covalently linked to the second component) may be pre-complexed with a HA (i.e., a third component). In some of these embodiments, the first, second, and third components form a conjugate.

Provided herein are non-limiting examples of first, second, and third components.

### A. First Components - Therapeutically Active Agents

In many embodiments, the first component is capable of binding to a therapeutic target, which makes it a biologically active or therapeutically active agent. In some embodiments, the first component is capable of binding to a therapeutic target in the eye. The term “capable of binding” as used herein means that a substance or agent or component can specifically bind to a target and optionally modulate the activity of the target. In other words, a first component is therapeutically active in the eye as a consequence of its binding to the therapeutic target in the eye. In some embodiments, the first component may activate, inactivate, increase, or decrease activity of the therapeutic target after binding to it. In some embodiments, the therapeutic target is a suitable structure in the eye, the activity of which is associated with an eye disease to be treated. In some embodiments, the first component binds to a component directly upstream or downstream of a therapeutic target in a signal transduction cascade. In some embodiments, the first component comprises a known therapeutic drug for treatment of an eye disease.

A specific binding component or binding domain has preferably at least an affinity of 10⁶ l/mol for its corresponding target molecule. The specific binding domain preferably has an affinity of 10⁷ l/mol or even more preferred of 10⁸ l/mol or most preferred of 10⁹ l/mol for its target molecule. “Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art. As the skilled artisan will appreciate, the term “specific” is used to indicate that other biomolecules present do not significantly bind to the binding agent specific for the binding domain. Preferably, the level of binding to a biomolecule other than the target molecule results in a binding affinity which is only 10% or less, more preferably only 5% or less of the affinity to the target molecule, respectively. A preferred specific binding agent will fulfill both the above minimum criteria for affinity as well as for specificity.

In some embodiments, the first component comprises a protein such as a receptor or a fragment thereof that binds a therapeutic target, an antibody or fragments thereof, a growth factor, a cysteine knot peptide, an enzyme, or a DARpin. In some embodiments, the protein may range in size from small to large. In some embodiments, the protein is a peptide comprising two to twenty amino acids. In some embodiments, the protein is a polypeptide comprising twenty-one to fifty amino acids. In some embodiments, the protein is a polypeptide comprising more than fifty amino acids. In some embodiments, the protein is a protein complex comprising two or more linear chains or amino acids wherein each amino acid chain may comprise any number of amino acids. In some embodiments, the first component is no greater than 80 kDa. In some embodiments, the first component is greater than 80 kDa.

In some embodiments, the first component comprises a nucleic acid which may be DNA or RNA. The nucleic acid may be complementary to the nucleic acid relating to the target (e.g., a nucleic acid complementary to a target’s mRNA or relevant part thereof). In some embodiments, the nucleic acid is an aptamer. In some embodiments, the nucleic acid comprises an antisense oligonucleotide. In some embodiments, the nucleic acid comprises a locked nucleic acid.

### 1. Therapeutic Targets in the Eye

In some embodiments, the first component binds to a therapeutic target in the eye. There are many therapeutic targets in the eye. As therapies are developed that effectively target these molecules and pathways, there will be a need to provide the improvements in visual outcomes while reducing the treatment burden and risks associated with frequent IVT injections.

### a) Proangiogenic, Inflammatory, and Growth Factor Mediators

In some embodiments, the first component binds to a therapeutic target that is a proangiogenic, inflammatory, and/or growth factor mediator. Proangiogenic, inflammatory, and growth factor mediators are involved in the retinal diseases, such as, for example, neovascular age-related macular degeneration (AMD; wet AMD), diabetic retinopathy, and retinal vein occlusions.

Examples of these proangiogenic, inflammatory, or growth factor mediator molecules include but are not limited to plate-derived growth factor (PDGF), angiopoietin, S1P, integrin αvβ3, integrin αvβ5, integrin α5β1, betacellulin, apelin/APJ, erythropoietin, complement factor D, and TNFα.

### b) Proteins in Age-Related Macular Degeneration (AMD)

In some embodiments, the first component binds to a protein that is genetically linked to increased risk in age-related macular degeneration (AMD) risk. In some embodiments, the first component binds to a complement pathway component such as C2, factor B, factor H, CFHR3, C3b, C5, C5a, and C3a. In some embodiments, the first component binds to HtrA1, ARMS2, TIMP3, HLA, IL-8, CX3CR1, TLR3, TLR4, CETP, LIPC, or COL10A1.

### c) Vascular Endothelial Growth Factor (VEGF)

In some embodiments, the first component binds to vascular endothelial growth factor (VEGF). VEGF is known to be of relevance for a variety of eye diseases, e.g., conditions or disorders associated with diabetic retinopathy or with macular edema. (See Section III below.)

The term “VEGF” refers to the 165-amino acid vascular endothelial cell growth factor, and related 121-, 189-, and 206-amino acid vascular endothelial cell growth factors together with the naturally occurring allelic and processed forms of those growth factors. VEGF may refer to a VEGF protein from any species.

VEGF is essential in both normal developmental and pathologic angiogenesis. Hypoxia-induced secretion of VEGF by astrocytes is a key element that guides the formation of retinal vasculature. Elevated levels of VEGF also induce pathological outgrowth of new vessels in retina and choroid. Inhibition of angiogenic factors like VEGF has become a major strategy in designing therapeutic approaches for treatment of pathological ocular angiogenesis, including age-related macular degeneration, proliferative retinopathy and retinopathy of prematurity.

The term “VEGF-mediated disorder” refers to any disorder, the onset, progression or the persistence of the symptoms or disease states of which requires the participation of VEGF. Exemplary VEGF-mediated disorders include, but are not limited to, age-related macular degeneration, neovascular glaucoma, diabetic retinopathy, macular edema, diabetic macular edema, pathologic myopia, retinal vein occlusions, retinopathy of prematurity, abnormal vascular proliferation associated with phacomatoses, edema (such as that associated with brain tumors), Meigs’ syndrome, rheumatoid arthritis, psoriasis and atherosclerosis.

In some embodiments, the first component is a VEGF receptor such as VEGFR1, VEGFR2, VEGFR3, mbVEGFR, or sVEGFR.

In some embodiments, the first component is an antibody or antigen-binding fragment against VEGF, more particularly an anti-VEGF Fab. VEGF antibodies and antigen-binding fragments are provided by the disclosure herein. Other anti-VEGF antibodies, VEGF antagonists, and VEGF receptor antagonists that can be used include, for example: ranibizumab, bevacizumab, aflibercept, pegaptanib, CT-322 and anti-VEGF antibodies and fragments as discussed in US 2012/0014958, WO 1998/045331, and WO 2015/198243, which are incorporated herein by reference in their entireties. In some embodiments, the first component comprises a drug that targets VEGF, such as those disclosed in Section II.A.2.a) below.

### d) Erythropoietin (EPO)

In some embodiments, the first component binds to erythropoietin (EPO). In some embodiments, the first component binds to erythropoietin receptor (EPOR). EPO refers to the erythropoietin protein in different species. The protein sequences for human, cynomolgus, mouse, rat, and rabbit EPO are publicly available. Human EPO can also be hyperglycosylated. The terms “EPO Receptor” or “EPOR” are used interchangeably and refer to the erythropoietin receptor protein in different species.

### e) Angiopoietin

In some embodiments, the first component binds to an angiopoietin such as angiopoietin 2 (ANG2). ANG2 is known as therapeutic candidate for wet AMD as it functions in both angiogenesis and immune activation, two processes that are involved in ocular pathological neovascularization. In human eyes, higher levels of ANG2 correlate with disease severity in wet AMD. Increased intraocular ANG2 levels were also detected in patients with diabetic retinopathy and retinal vein occlusion, indicating a potential medical significance of targeting ocular ANG2. ANG2 refers to protein in different species. It has also been suggested to use combined inhibition of VEGF-A/ANG2 to strongly reduced vascular leakage, immune reactivity, and apoptosis.

### f) Interleukins

In some embodiments, the first component binds to an interleukin, such as Interleukin (IL-) 1β (IL-1β), IL-6, IL-10, IL-17A, and IL-19. Interleukins have been associated with eye diseases such as uveitis, a potentially blinding inflammatory disease. The interleukins may be of any species.

### g) Platelet-derived Growth Factor (PDGF)

In some embodiments, the first component binds to a therapeutic target that is platelet-derived growth factor (PDGF) or platelet-derived growth factor subunit B (PDGF-BB). The PDGF and PDGF-BB may be derived from any species. In some embodiments, the first component comprises a PDGF antagonist, such as those disclosed in Section II.A.2.e) below.

### h) VPDF

In some embodiments, the first component binds to VEGF and PDGF. A variety of proteins, antibodies, antibody fragments, binding domains, agonists, and antagonists may bind to VEGF and PDGF. As used herein, the term “anti-VP” refers to a bispecific antibody or fragment thereof that binds to VEGF and PDGF.

In some embodiments, the first component is a dual-targeting Fab, i.e., a dutaFab. As used herein, “anti-VPDF” refers to a dutaFab that binds to VEGF and PDGF.

### i) HtrA Proteins

In some embodiments, the first component binds to a member of the HtrA family of serine proteases. HtrA proteins have a catalytic domain with at least one C-terminal PDZ domain, and ATP-independent proteinase chaperones related to protein metabolism and cell fate. Clausen et al., Molecular cell 10(3):443-445 (2002). There are four HtrA proteins in humans: HtrA1, HtrA2, HtrA3, and HtrA4. In humans, HtrA1, HtrA3, and HtrA4 share the same domain architecture: an N-terminal IGFBP-like module and a Kazal-like module, a protease domain with trypsin-like fold, and a C-terminal PDZ domain. human genetic studies have identified a strong correlation between progression of age-related macular degeneration (AMD) and a single nucleotide polymorphism (SNP) in the HtrA1 promoter region which results in increased HtrA1 transcript levels. Dewan et al., Science 314:989-992 (2006); Yang et al., Science 314:992-933 (2006).

In some embodiments, the first component binds to HtrA1. In some embodiments, the first component binds to HtrA2. In some embodiments, the first component binds to HtrA3. In some embodiments, the first component binds to HtrA4.

### j) Other Therapeutic Targets

In some embodiments, the first component binds to one of the following therapeutic targets: Factor P, Factor D, TNFα, FGFR, IL-6R, Tie2, S1P, integrin αvβ3, integrin αvβ5, integrin α5β1, betacellulin, apelin/APJ, complement factor D, TNFα, HtrA1, ST-2 receptor, insulin, human growth factor, complement factor H, CD35, CD46, CD55, CD59, complement receptor 1 -related (CRRY), nerve growth factor, pigment epithelium-derived factor, endostatin, ciliary neurotrophic factor, complement factor 1 inhibitor, complement factor like-1, complement factor I, or the like.

The term “Factor D” refers to the Factor D protein derived from any species.

The term “Factor P” refers to the Factor P protein derived from any species. Human Factor P can be obtained from Complement Tech, Tyler, TX. Cynomolgus Factor P can be purified from cynomolgus serum (protocol adapted from Nakano et al., 1986, J Immunol Methods 90:77-83. Factor P is also known in the art as “Properdin.”

The term “FGFR2” refers to fibroblast growth factor receptor 2 derived from any species.

### 2. Therapeutic Drugs

Any suitable therapeutic agent for the treatment of an eye disease can be used as a first component, (which are discussed in Section III below). In some embodiments, the first component comprises an acknowledged therapeutic drug that binds to a target in the eye. In some embodiments, the first component binds to a target of human origin. In some embodiments, the first component comprises an acknowledged therapeutic drug for treatment an eye disease.

### a) Drugs That Target VEGF

In some embodiments, the first component comprises a VEGF antagonist, including, for example, but not limited to: (1) an anti-VEGF antibody (e.g., LUCENTIS® (ranibizumab), RTH-258 (formerly ESBA-1008, an anti-VEGF single-chain antibody fragment; Novartis), or a bispecific anti-VEGF antibody (e.g., an anti-VEGF/anti-angiopoeitin 2 bispecific antibody such as RG-7716; Roche)), (2) a soluble VEGF receptor fusion protein (e.g., EYLEA®; aflibercept)), (3) an anti-VEGF DARPin® (e.g., abicipar pegol; Molecular Partners AG/Allergan), or (4) an anti-VEGF aptamer (e.g,. MACUGEN®; pegaptanib sodium)).

In some embodiments, the first component comprises LUCENTIS® (ranibizumab), particularly for treatment of an eye disease. In some instances, the eye disease is age-related macular degeneration (AMD; e.g., wet AMD). In some instances, the eye disease is geographic atrophy (GA). In some instances, the eye disease is diabetic macular edema (DME) and/or diabetic retinopathy (DR; e.g., non-proliferative DR (NPDR) or proliferative DR (PDR)).

In some embodiments, the first component comprises RTH-258, particularly for treatment of an eye disease. In some instances, the eye disease is AMD (e.g., wet AMD). In some instances, the eye disease is GA.

In some embodiments, the first component comprises EYLEA® (aflibercept), particularly for treatment of an eye disease. In some instances, the eye disease is AMD (e.g., wet AMD). In some instances, the eye disease is GA. In some instances, the eye disease is DME and/or DR (e.g., NPDR or PDR).

In some embodiments, the first component comprises abicipar pegol, particularly for treatment of an eye disease. In some instances, the eye disease is AMD (e.g., wet AMD). In some instances, the eye disease is GA.

In some embodiments, the first component comprises MACUGEN® (pegaptanib sodium), particularly for treatment of an eye disease. In some instances, the eye disease is AMD (e.g., wet AMD). In some instances, the eye disease is GA.

### b) Anti-Angiogenic Agents

In some embodiments, the first component comprises an anti-angiogenic agent. Non-limiting examples of anti-angiogenic agents include anti-VEGF antibodies (e.g., the anti-VEGF Fab LUCENTIS® (ranibizumab), RTH-258 (formerly ESBA-1008, an anti-VEGF single-chain antibody fragment; Novartis), bispecific anti-VEGF antibodies (e.g., an anti-VEGF/anti-angiopoeitin 2 bispecific antibody such as RG-7716; Roche), esoluble recombinant receptor fusion proteins (e.g., EYLEA® (aflibercept); also known as VEGF Trap Eye; Regeneron/Aventis), VEGF variants, soluble VEGF receptor (VEGFR) fragments, aptamers capable of blocking VEGF (e.g., the anti-VEGF pegylated aptamer MACUGEN® (pegaptanib sodium; NeXstar Pharmaceuticals/OSI Pharmaceuticals)), aptamers capable of blocking VEGFR, neutralizing anti-VEGFR antibodies, small molecule inhibitors of VEGFR tyrosine kinases, anti-VEGF DARPin® (e.g., abicipar pegol; Molecular Partners AG/Allergan), small interfering RNAs which inhibit expression of VEGF or VEGFR, VEGFR tyrosine kinase inhibitors (e.g., 4-(4-bromo-2-fluoroanilino)-6-methoxy-7-(1-methylpiperidin-4-ylmethoxy)quinazoline (ZD6474), 4-(4-fluoro-2-methylindol-5-yloxy)-6-methoxy-7-(3-pyrrolidin-1-ylpropoxy)quinazoline (AZD2171), vatalanib (PTK787), semaxaminib (SU5416; SUGEN), and SUTENT® (sunitinib)), and combinations thereof.

### c) Anti-Neovascularization Agents

In some embodiments, the first component comprises an agent that has activity against neovascularization for treatment of an eye disease, such as an anti-inflammatory drug, a mammalian target of rapamycin (mTOR) inhibitor (e.g., rapamycin; AFINITOR® (everolimus), and TORISEL® (temsirolimus)), cyclosporine, a tumor necrosis factor (TNF) antagonist (e.g., an anti-TNFα antibody or antigen-binding fragment thereof (e.g., infliximab, adalimumab, certolizumab pegol, and golimumab) or a soluble receptor fusion protein (e.g., etanercept)), an anti-complement agent, a nonsteroidal anti-inflammatory agent (NSAID), or combinations thereof.

### d) Neuroprotective Agents

In some embodiments, the first component comprises an agent that is neuroprotective and can potentially reduce the progression of disease. For example, said agent may reduce progression of dry AMD to wet AMD. Examples of neuroprotective agents include the class of drugs called the “neurosteroids,” which include drugs such as dehydroepiandrosterone (DHEA) (PRASTERA™ and FIDELIN®), dehydroepiandrosterone sulfate, and pregnenolone sulfate.

### e) PDGF Antagonists

In some embodiments, the first component comprises a PDGF antagonist. In some embodiments, the PDGF antagonist is (1) an anti-PDGF antibody (e.g., REGN2176-3), (2) an anti-PDGF-BB pegylated aptamer (e.g., FOVISTA®; E10030; Ophthotech/Novartis), (3) a soluble PDGFR receptor fusion protein, (4) a dual PDGF/VEGF antagonist/inhibitor (e.g., DE-120 (Santen) or X-82 (TyrogeneX)), (5) a bispecific anti-PDGF/anti-VEGF antibody), (6) an anti-PDGFR antibody, or (7) a small molecule inhibitor (e.g., squalamine).

### f) Complement System Antagonists

In some embodiments, the first component comprises a complement system antagonist. Examples of complement system antagonists include complement factor C5 antagonists (e.g., a small molecule inhibitor (e.g., ARC-1905; Opthotech)), anti-C5 antibodies (e.g., LFG-316; Novartis), properdin antagonists (e.g., an anti-properdin antibody; CLG-561; Alcon), complement factor D antagonists (e.g., an anti-complement factor D antibody; lampalizumab; Roche), and C3 blocking peptides (e.g., APL-2; Appellis).

### g) Acknowledged Drugs for Treatment of Eye Diseases

In some embodiments, the first component comprises an acknowledged therapeutic drug for treatment of an eye disease. Treatments of eye diseases are discussed in Section III below. Examples of acknowledged drugs include nonsteroidal anti-inflammatory drugs (NSAIDs), steroids (e.g., for reduction of inflammation and/or fibrosis), antibiotics, topical ophthalmic anesthetics, ocular adhesives (e.g., for post-surgical wound closure), enzymatic agents (for vitreous surgery), DNA or RNA e.g. for gene therapy technologies, agents mediating neuroprotective effects, such as supplying neurotrophins, blocking excess glutamate stimulation, stabilizing Ca²⁺ homeostasis, preventing apoptosis, modulating immunologic status via vaccination, inducing endogenous neuro-protective mechanisms, antioxidants, vitamins, and mineral supplements.

In some embodiments, the first component comprises any suitable DME and/or DR therapeutic agent, particularly for treatment of an eye disease, including, but not limited, to a VEGF antagonist (e.g., LUCENTIS® or EYLEA®), a corticosteroid (e.g., a corticosteroid implant, OZURDEX®; dexamethasone IVT implant; or ILUVIEN®, fluocinolone acetonide IVT implant) or a corticosteroid formulated for administration by IVT injection (e.g., triamcinolone acetonide), or combinations thereof. In some instances, the eye disease is DME and/or DR.

Further examples of acknowledged drugs for treatment of eye diseases that are suitable for use as first components conditions include, but are not limited to, VISUDYNE® (verteporfin; a light-activated drug that is typically used in conjunction with photodynamic therapy with a non-thermal laser), PKC412, Endovion (NS 3728; NeuroSearch A/S), neurotrophic factors (e.g., glial derived neurotrophic factor (GDNF) and ciliary neurotrophic factor (CNTF)), diltiazem, dorzolamide, PHOTOTROP®, 9-cis-retinal, eye medication (e.g., phospholine iodide, echothiophate, or carbonic anhydrase inhibitors), veovastat (AE-941; AEterna Laboratories, Inc.), Sirna-027 (AGF-745; Sima Therapeutics, Inc.), neurotrophins (including, by way of example only, NT-⅘, Genentech), Cand5 (Acuity Pharmaceuticals), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celltech Group plc), ATX-S10 (Hamamatsu Photonics), TGF-beta 2 (Genzyme/Celtrix), tyrosine kinase inhibitors (e.g., those discussed in U.S. Pat. No. 7,771,742, and VEGFR inhibitors SUGEN (SU5416) and Pfizer’s Inlyta, dacomitinib, LORBRENA® (lorlatinib), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), cyclosporin A, therapeutic agents used in photodynamic therapy (e.g., VISUDYNE®; receptor-targeted PDT, Bristol-Myers Squibb, Co.; porfimer sodium for injection with PDT; verteporfin, QLT Inc.; rostaporfin with PDT, Miravent Medical Technologies; talaporfin sodium with PDT, Nippon Petroleum; and motexafin lutetium, Pharmacyclics, Inc.), antisense oligonucleotides (including, by way of example, products tested by Novagali Pharma SA and ISIS-13650, Ionis Pharmaceuticals), and combinations thereof.

In some embodiments, the first component comprises a tissue factor antagonist (e.g., hI-con1; Iconic Therapeutics), an alpha-adrenergic receptor agonist (e.g., brimonidine tartrate; Allergan), a peptide vaccine (e.g., S-646240; Shionogi), an amyloid beta antagonist (e.g., anti-beta amyloid monoclonal antibody; GSK-933776), an S1P antagonist (e.g., anti-S1P antibody; iSONEP™; Lpath Inc), a ROBO4 antagonist, an anti-ROBO4 antibody (e.g., DS-7080a; Daiichi Sankyo).

In some embodiments, the first component comprises Tryptophanyl-tRNA synthetase (TrpRS), squalamine, RETAANE® (anecortave acetate for depot suspension; Alcon, Inc.), Combretastatin A4 Prodrug (CA4P), MIFEPREX® (mifepristone-ru486), subtenon triamcinolone acetonide, IVT crystalline triamcinolone acetonide, matrix metalloproteinase inhibitors (e.g., Prinomastat; AG3340; Pfizer), fluocinolone acetonide (including fluocinolone intraocular implant; Bausch & Lomb/Control Delivery Systems), linomide, inhibitors of integrin β3 function, angiostatin, and combinations thereof. These and other therapeutic agents are described, for example, in U.S. Pat. Application No. US 2014/0017244, which is incorporated herein by reference in its entirety.

### 3. Antibodies and Antigen-binding Fragments

In some embodiments, the first component comprises or is derived from an antibody or antigen-binding fragment thereof that is capable of binding an antigen. The extent of the antibody or antigen-binding fragment’s binding to an unrelated, non-target protein is less than about 10% of the binding of the antibody to the target as measured, e.g., by surface plasmon resonance (SPR). In certain aspects, an antibody or antigen-binding fragment that binds to the target has a dissociation constant (K_{D}) of ≤ 1 µM, ≤ 100 nM, ≤ 10 nM, ≤ 1 nM, ≤ 0.1 nM, ≤0.01 nM, or ≤ 0.001 nM (e.g., 10⁻⁸ M or less, e.g., from 10⁻⁸ M to 10⁻¹³ M, e.g., from 10⁻⁹ M to 10⁻¹³ M). An antibody or antigen-binding fragment thereof is said to “specifically bind” to the target when the antibody has a K_{D} of 1 µM or less.

In some embodiments, the antibody or antigen-binding fragment thereof comprises a bispecific antibody, an antibody lacking at least the Fc domain, a Fab fragment, a (Fab′)₂ fragment, a Fab′ fragment, VhH fragment, scFv fragment, scFv-Fc fragment, or minibody.

In some embodiments, the antibody or antigen-binding fragment thereof binds to an antigen that is present in the eye. In some embodiments, the antibody or antigen-binding fragment thereof may bind to VEGF, HtrA1, IL-33, C5, Factor P, Factor D, EPO, EPOR, IL-1β, IL-17A, IL-10, TNFα, FGFR2, PDGF, or ANG2.

In some embodiments, the first component is an anti-VEGF antibody or antibody-binding fragment, an anti-PDGF antibody or antibody-binding fragment, an anti-ANG2 antibody or antibody-binding fragment, or an anti-IL-1β antibody or antibody-binding fragment. Examples of antibodies that bind VEGF include Lucentis® (ranibizumab), Eylea® (aflibercept), Beovu® (brolucizumab-dbll), and Avastin® (bevacizumab).

In some embodiments, the antibody comprises a bispecific antibody. In some embodiments, the bispecific antibody is an anti-VEGF/anti-Ang2 bispecific antibody, such as RG-7716 or any bispecific anti-VEGF/anti-Ang2 bispecific antibody disclosed in WO 2010/069532 or WO 2016/073157 or a variant thereof. In some embodiments, the bispecific antibody is an anti-VPDF antibody, i.e., an anti-VEGF and anti-PDGF dutaFab antibody.

In some embodiments, the first component is an anti-IL-6 antibody, for example, EBI-031 (Eleven Biotherapeutics; see, e.g., WO 2016/073890), siltuximab (SYLVANT®), olokizumab, clazakizumab, sirukumab, elsilimomab, OPR-003, MEDI5117, PF-04236921, or a variant thereof.

In some embodiments, the first component is an anti-IL-6R antibody, for example, tocilizumab (ACTEMRA®) (see, e.g., WO 1992/019579), sarilumab, ALX-0061, SA237, or a variant thereof.

In some embodiments, the first component is RabFab, an antigen-binding Fab fragment derived from a parent monoclonal antibody (G10) raised in rabbits against a phosphorylated peptide derived from the intracellular domain of the human cMET receptor and as such does not bind an extracellular target in the eye. Shatz, W. et al., Mol. Pharm., 13(9):2996-3003 (2016).

In some embodiments, the antigen-binding fragment comprises a peptide or a polypeptide that is not an antibody or an antigen-binding fragment thereof.

### 4. Growth Factors

In some embodiments, the first component comprises a growth factor. In some embodiments, the growth factor comprises fibroblasts growth factors, platelet-derived growth factors, nerve growth factor (NGF), VEGF, fibroblast growth factor (FGF), and insulin-like growth factor-I (IGF-I).

### 5. Cysteine Knot Peptides

In some embodiments, the first component comprises a cysteine knot peptide. In some embodiments, the cysteine knot peptide comprises at least 90%, 91%, 92%, 93%, 94%, 95%, 96%, 97%, 98%, 99%, or 100% sequence identity with SEQ ID NO: 92 (cysteine knot peptide sequence).

A cysteine knot peptide may be covalently linked to another molecule to form a first component, including any of the exemplary first components discussed in Section II.A.2 above through Section II.A.4 above. In some embodiments, the first component comprises a cysteine knot peptide that is covalently linked to an anti-VEGF antigen-binding fragment.

In some embodiments, the HABD (i.e., second component) is covalently linked to the first component at the cysteine knot peptide. In some embodiments, the covalent linker comprises at least 90%, 91%, 92%, 93%, 94%, 95%, 96%, 97%, 98%, 99%, or 100% sequence identity with SEQ ID NO: 95. In some embodiments, the covalent linker comprises the sequence GSGSGSGSGSGSGSGSGSGS (SEQ ID NO: 95). In some embodiments, the cysteine knot peptide is covalently linked to VG1 with a C-terminal his-tag (SEQ ID NO: 29). In some embodiments, the cysteine knot peptide is covalently linked to VG1 with an Ig domain deletion and a C-terminal his-tag (SEQ ID NO: 32). In some embodiments, the cysteine knot peptide is covalently linked to VG1 with an N-terminal his-tag. In some embodiments, the cysteine knot peptide is covalently linked to VG1 with an Ig domain deletion and an N-terminal his-tag.

### B. Second Components - Hyaluronan-Binding Domains (HABDs)

In many embodiments, the second component comprises or is derived from a HA-binding protein (which comprises a HA-binding domain; HABD). In some embodiments, the second component comprises a HABD. Examples of proteins that comprise HABDs include CD44, tumor necrosis factor-stimulated gene-6 (TSG6), Versican, brain-specific link protein (BRAL1), Lymphatic Vessel Endothelial Hyaluronan Receptor-1 (LYVE-1), and Aggrecan.

In some embodiments, the two second components may be different or identical. For example, the therapeutic molecule may comprise as second components two CD44 domains, two TSG-6 domains, two VG1 domains, or any combination of the foregoing domains to make a pair of two different domains.

The eye is a complex tissue that has several distinct compartments including the cornea, aqueous humor, lens, vitreous humor, retina, the retinal pigment epithelium, and choroid. The compartments include extracellular macromolecules such as HA.

The term “hyaluronan-binding protein” or “HA-binding protein” refers to a protein or a family of proteins that bind HA. Typically, these HA-binding proteins comprise HABDs. Various HA-binding molecules are well-known in the art, which may be used as a second component (see e.g., Day, et al., 2002, J Bio. Chem 277: 4585 and Yang et al., 1994, EMBO J 13: 286-296). Exemplary HA-binding proteins include CD44, LYVE-1, Aggrecan, Versican, Brevican, Neurocan, Hyaluronan binding protein 1 (HABP1; also known as C1qBP/C1qR and p32), HAPLN1 (also known as link protein and CRTL1), Hyaluronan and Proteoglycan Link Protein 4 (HAPLN4; also known as brain link protein 2), Layilin, Stabilin-1, Stabilin-2, brain-specific link protein (BRAL1), or tumor necrosis factor-stimulated gene-6 (TSG-6), RHA M, bacterial HA synthase, and collagen VI.

Many HA-binding proteins, and peptide fragments, contain a common structural domain of about 100 amino acids in length involved in HA-binding; the structural domain is referred to as a “LINK Domain” (Yang et al., EMBO J 13:2, 286-296 (1994) and Mahoney et al., J Bio. Chem 276:25, 22764-22771 (2001)). Any such protein may be used in the present invention. The HABD of any HA-binding protein, such as the above exemplary proteins may be comprised in the second component to confer capability of binding to HA. Preferably, the second component comprises a CD44 (CD44) domain, a brain-specific link protein (BRAL1) domain, a tumor necrosis factor-stimulated gene-6 (TSG-6) domain, a Lymphatic Vessel Endothelial Hyaluronan Receptor-1 (LYVE-1) domain, a Hyaluronan Binding Protein (HABP) domain, an Aggrecan G1 (AG1) domain or a Versican G1 (VG1) domain. Exemplary and suitable HA-binding molecules, including peptide tags, for use in the eye are described in WO 2014/99997 and WO 2015/19824, and are incorporated by reference in their entireties. Any of the sequences described therein may be used in the present invention.

In some embodiments, the second component is covalently linked to the first component in order to decrease the clearance of the first component from the eye, thereby increasing its ocular half-life. The first components may benefit from having longer ocular retention and/or a longer duration of action in eye disease.

Additionally, the second component may be non-covalently bound to the third component comprising HA to form a conjugate. In some embodiments, each second component in a conjugate may bind to separate molecules of HA. In some embodiments, two or more second components may bind to the same HA molecule.

In many embodiments, the binding affinity of the HABD for HA may fall within several ranges; the binding affinity may be modulated depending on the mechanism of action of the therapeutically active agent. For example, if the site of action is in the vitreous humor, high binding affinity may help keep the biological agent within the vitreous humor. If the site of action is in the retina instead, lower binding affinity may help the biological agent traverse the vitreous humor to arrive at the retina.

In many embodiments, the HABD has a binding affinity for HA that can be measured using methods that comprise surface plasmon resonance (SPR). Without being bound by theory, in some embodiments, the binding affinity (K_{D}) ranges of the HABD for HA comprises 10 nM to 10 µM, 5 nM to 10 nM, and 100 nM to 5 µM.

In many embodiments, the HABD’s interaction with HA can be observed. In some embodiments, the interaction is observed using methods that comprise fluorescence correlations spectroscopy (FCS). In FCS, diffusion of molecules can be determined by monitoring the fluorescence intensity in a small volume portion of a solution. The fluorescence intensity fluctuates due to the movement of molecules and quantitative analysis of these fluctuations can yield diffusion times for the molecules. By employing a fluorescent dye with appropriate spectroscopic properties, diffusion in biological matrices can be determined. In some embodiments, the observations by FCS correlate with the measurements by SPR.

In some embodiments, the HABD comprises a sequence that is wild type when compared to its protein of origin. In some embodiments, the HABD may comprise one or more mutations in its protein sequence when compared to its protein of origin. In many embodiments, these mutations comprise single amino acid substitutions, double amino acid substitutions, additions, deletions, and truncations.

In some embodiments, the HABD comprises single or double amino acid substitutions. In many examples, the substitution may comprise conservative a mutation wherein an amino acid replacement changes the original amino acid to a different amino acid with similar biochemical properties. In other examples, the substitution may comprise a non-conservative mutation wherein an amino acid replacement changes the original amino acid to a different amino acid with different biochemical properties.

In some embodiments, the HABD comprises amino acids that contribute to HA binding. In some embodiments, these amino acids may be conserved to maintain HA binding affinity. In some embodiments, these amino acids may be substituted to alter HA binding affinity, depending on the affinity desired and the duration desired for the long acting therapeutic.

In some embodiments, the HABD comprises amino acids that contribute to thermostability of the HABD and or the therapeutic molecule. In some embodiments, these amino acids may be conserved to main thermostability. In some embodiments, these amino acids may be substituted to alter thermostability.

In some embodiments, the HABD comprises at least 1, at least 2, at least 3, at least 4, or at least 5 mutations relative to one of the reference sequences disclosed herein. In some embodiments, the HABD comprises 1 to 3 mutations, wherein the 1 to 3 mutations independently comprise single amino acid substitutions, double amino acid substitutions, additions, deletions, and truncations. In some embodiments, the HABD comprises 1 to 5 mutations, wherein the 1 to 5 independently mutations comprise single amino acid substitutions, double amino acid substitutions, additions, deletions, and truncations.

In some embodiments, the second component comprises or is derived from CD44, TSG6, or Versican. In some embodiments, the second component comprises a CD44 domain, a TSG6 domain, or a Versican domain.

### 1. CD44

In some embodiments, the second component is derived from CD44 (SEQ ID NO: 1). The CD44 receptor comprises a LINK domain, GAG attachment domain, transmembrane domain, and a cytoplasmic domain. Several isoforms with different modular compositions that are processed by alternative splicing are described. In some embodiments, the second component is derived from or comprises the CD44 HA receptor domain. In some embodiments, the second component is derived from or comprises SEQ ID NO: 2.

### 2. Tumor Necrosis Factor-Stimulated Gene-6 (TSG6)

In some embodiments, the second component is derived from TSG6. TSG-6, also known as TNFAIP6, is comprised of an HA-binding link domain followed by a CUB domain. In some embodiments, the second component is derived from or comprises the TSG6 HA binding link domain. In some embodiments, the second component is derived from or comprises SEQ ID NO: 4.

### 3. Versican

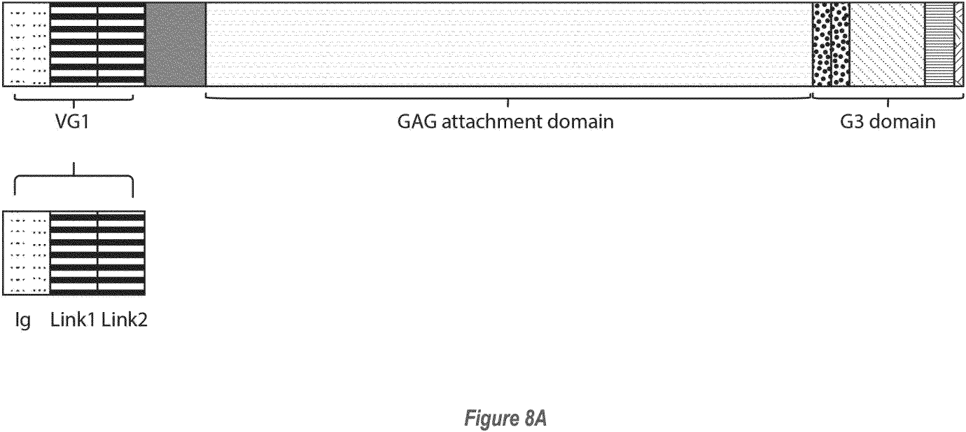

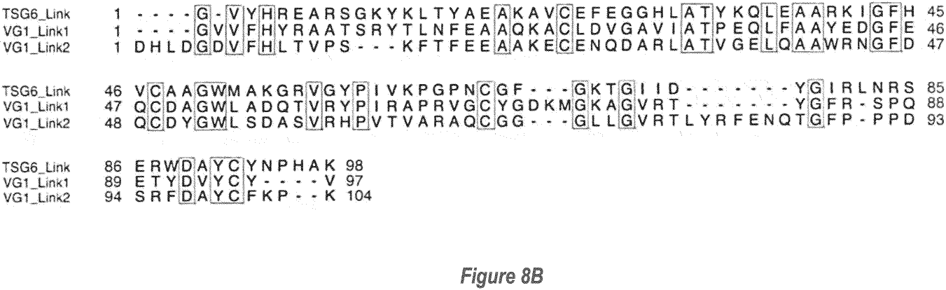

In some embodiments, the second component is derived from Versican. Versican comprises the following domains: VG1, GAG attachment domain, and G3 domain (FIG. 8A). The VG1 domain (SEQ ID NO: 29) comprises Ig domain, Link1, and Link2 (FIG. 8A). In some embodiments, the second component comprises Link1 (SEQ ID NO: 30) and/or Link2 (SEQ ID NO: 31), wherein Link1 and/or Link2 are capable of binding HA.

### a) Wild Type VG1

In some embodiments, the HABD comprises wild type (WT) VG1 with an amino acid sequence as set forth in SEQ ID NO: 29. In some embodiments, the HABD comprises an amino acid sequence as set forth in Link1 (SEQ ID NO: 30) and/or Link2 (SEQ ID NO: 31).

### b) Mutant VG1

In some embodiments, the HABD comprises mutant VG1. In many embodiments, the VG1 mutations are relative to the amino acid sequences as set forth in SEQ ID NO: 29 (WT VG1), 32 (VG1ΔIg), 60 (WT VG1 consensus sequence), or 86 (VG1ΔIg consensus sequence). In some embodiments, the HABD comprises a sequence at least 90, 91, 92, 93, 94, 95, 96, 97, 98, 99, or 100% identical to SEQ ID NO: 29 (WT VG1), 32 (VG1ΔIg), 60 (WT VG1 consensus sequence), or 86 (VG1ΔIg consensus sequence). In some embodiments, the HABD comprises a sequence at least 95% identical to SEQ ID NO: 29 (WT VG1), 32 (VG1ΔIg), 60 (WT VG1 consensus sequence), or 86 (VG1ΔIg consensus sequence).

### c) Truncated VG1

In some embodiments, the HABD comprises a truncation mutation relative to SEQ ID NO: 29 (WT VG1) or 60 (WT VG1 consensus sequence). In some embodiments, the HABD comprises a truncation of from 1 to 129 amino acids from the N-terminus of Versican. In some embodiments, the HABD comprises a truncated sequence wherein the Ig domain of wild type Versican is absent. In some embodiments, the HABD comprises a sequence at least 90, 91, 92, 93, 94, 95, 96, 97, 98, 99, or 100% identical to SEQ ID NO: 32 (VG1ΔIg) or 86 (VG1ΔIg consensus sequence). In some embodiments, the HABD comprises a sequence at least 95% identical to SEQ ID NO: 32 (VG1ΔIg) or 86 (VG1ΔIg consensus sequence). In some embodiments, the HABD comprises SEQ ID NO: 32 (VG1ΔIg).

### d) Amino Acid Substitutions

In some embodiments, the HABD comprises at least one of the following amino acids relative to SEQ ID NO: 29: R160, Y161, E194, D197, Y208, R214, Y230, F261, D295, and R233. In some embodiments, the HABD comprises 2, 3, 4, 5, 6, 7, 8, 9, or 10 of the following amino acids relative to SEQ ID NO: 29: R160, Y161, E194, D197, Y208, R214, Y230, F261, D295, and R233.

In some embodiments, the HABD comprises a sequence with amino acids that may be mutated relative to wild type to increase or decrease HA binding affinity. In some embodiments, the HABD comprises a mutation in at least one of the following positions relative to SEQ ID NO: 29: R160, Y161, E194, D197, Y208, R214, M222, Y230, R233, K260, F261, D295, Y296, H306, R312, L325, Y326, and R327. In some embodiments, the HABD comprises a mutation in 2, 3, 4, 5, 6, 7, 8, 9, 10, 11, 12, 13, 14, 15, 16, 17, or 18 of the following positions relative to SEQ ID NO: 29: R160, Y161, E194, D197, Y208, R214, M222, Y230, R233, K260, F261, D295, Y296, H306, R312, L325, Y326, and R327. In some embodiments, the HABD comprises a mutation in 2, 3, 4, 5, or 6 of the following positions relative to SEQ ID NO: 29: R160, Y161, E194, D197, Y208, R214, M222, Y230, R233, K260, F261, D295, Y296, H306, R312, L325, Y326, and R327.

In some embodiments, the HABD comprises at least one of the following mutations relative to SEQ ID NO: 29: R160A, Y161A, D197A, D197S, Y208A, Y208F, R214K, M222A, Y230A, Y230F, R233A, K260A, K260R, F261Y, KF260RY, D295A, D295S, Y296A, Y296F, DY295SF, H306A, R312A, L325A, Y326A, R327A, and LYR325LFK. In some embodiments, the HABD comprises at least one of Y208A and H306A.

In some embodiments, the HABD comprises at least 2, 3, 4, 5, 6, 7, 8, 9, 10, 11, 12, 13, 14, 15, 16, or 17 of the following mutations relative to SEQ ID NO: 29: R160A, Y161A, D197A, D197S, Y208A, Y208F, R214K, M222A, Y230A, Y230F, R233A, K260A, K260R, F261Y, KF260RY, D295A, D295S, Y296A, Y296F, DY295SF, H306A, R312A, L325A, Y326A, R327A, and LYR325LFK. In some embodiments, the HABD comprises at least 2, 3, 4, 5, or 6 of the following mutations relative to SEQ ID NO: 29: R160A, Y161A, D197A, D197S, Y208A, Y208F, R214K, M222A, Y230A, Y230F, R233A, K260A, K260R, F261Y, KF260RY, D295A, D295S, Y296A, Y296F, DY295SF, H306A, R312A, L325A, Y326A, R327A, and LYR325LFK.

In some embodiments, the HABD is SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or SEQ ID NO: 59.

### 4. Brain-Specific Link Protein (BRAL1)

In some embodiments, the second component is derived from BRAL1. BRAL1 comprises an immunoglobulin domain, link domain module 1, and link domain module 2. Link domain modules 1 and 2 are capable of binding HA. In some embodiments, the second component comprises a link domain link domain module 1 and/or link domain module 2 from BRAL1.

### 5. Lymphatic Vessel Endothelial Hyaluronan Receptor-1 (LYVE-1)

In some embodiments, the second component is derived from LYVE-1. LYVE-1 is a homolog of CD44 that comprises link domain that binds to HA. In some embodiments, the second component comprises a link domain from LYVE-1.

### 6. Aggrecan

In some embodiments, the second component is derived from Aggrecan. Aggrecan comprises three globular domains: the G1 domain has the structural motif of a link protein and interacts with HA, the G2 domain is homologous to the G1 domain and is involved in product processing, and the G3 domain makes up the carboxyl terminus of the core protein. In some embodiments, the second component comprises a G1 domain from Aggrecan.

### C. Third Components - Hyaluronan (HA)

In some embodiments, the therapeutic molecule further comprises one or more third components. In some embodiments, the third component comprises HA. In some embodiments, the therapeutic molecule (comprising first and second components) is pre-complexed with HA to form a conjugate. In some embodiments, the third component is a HA of a molecular weight of from 5 kDa to 20 kDa.

In some embodiments, the second component of the therapeutic molecule is non-covalently bound to the third component to form the conjugate. In some embodiments, the second component of the therapeutic molecule is covalently bound to the third component to form the conjugate.

Preferably, the second component covalently linked to the first component binds to the third component (i.e., hyaluronan) with a K_{D} of less than or equal to 10.0 µM. For example, the second component can bind HA with a K_{D} of less than or equal to 9.0 µM, 8.0 µM, 7.0 µM, 6.0 µM, 5.0 µM, 4.0 µM, 3.0 µM, 2.0 µM, 1.5 µM, 1.0 µM or 0.5 µM.

### 1. Hyaluronan (HA)

Hyaluronan (HA) is a linear glycosaminoglycan that occurs in extracellular matrix and on cell surfaces. HA contains repeating disaccharide units of N-acetyl glucosamine (GlcNac) and glucuronic acid (GlcUA), which are linked by alternating β1→3 glucuronidic and β1→4 glucosaminidic bonds, forming a linear polymer. HA is further described in Necas et al, 2008, Veterinarni Medicina, 53: 397-411. The glycosaminoglycan is ubiquitously present in the extracellular matrix of all vertebrates and is also present in the capsule of some strains of Streptococci. Functionally, HA molecules are important for the maintenance of a highly hydrated extracellular matrix in tissues, which is involved in cell adhesion and supports cell migration. The vitreous humour is besides of water primarily composed of HA, as it is excellent at retaining moisture and the structure in the central part of the eye. It helps to keep eyes lubricated and replenishes any moisture that is lost. HA also exhibits diverse biological functions by interacting with a large number of HA-binding proteins and cell surface receptors, such as CD44 and Lymphatic Vessel Endothelial Hyaluronan Receptor-1 (LYVE-1). Examples of HA-binding proteins and HABDs are discussed in Section II.B above.

HA has a wide molecular weight range from 1,000 to 10,000,000 Da. The native high molecular weight HA in tissues degrades into small molecules during the metabolic pathways through lymphatic system, lymph node, liver, and kidney. While the half-life of HA is known to be ca. 2.5 to 5.5 min in plasma, it was reported to be ca. 70 days in the vitreous body of eyes. The unique physicochemical properties and various biological functions of HA have led to its wide biomedical applications such as drug delivery, arthritis treatment, ocular surgery, and tissue engineering. In particular, HA has been investigated extensively for target-specific and long-term delivery of bio/pharmaceuticals through various delivery routes. Taking advantage of its viscoelastic and mucoadhesive properties, HA has been exploited as an effective delivery carrier of topical ophthalmic drugs.

It has been shown that HA of a defined size are particularly suitable in the present invention. In accordance with this the HA may have a molecular weight of at least 2, 3, 4, 5, 6, 7, 8, or 9 kDa and/or a molecular weight of at most 60, 50, 40, 30, 25, 20, or 15 kDa. Particularly suitable ranges for the molecular weight are of from 3 kDa to 60 kDa, particularly of from 4 kDa to 30 kDa, more particularly of from 5 kDa to 20 kDa.

In some embodiments, the use of unmodified naturally occurring HA is preferred. In these embodiments, the use of unmodified naturally occurring HA reduces side effects. For example, pre-complexation of a HABD with a HA of 10 kDa reduces in vitro precipitation in vitreous fluid and mitigates ocular toxicity observed in pigs and rabbits. In other examples, where the HABD is TSG-6 or CD44, ocular toxicity such as inflammation and retinal were observed when the TSG-6 or CD44 was not pre-complexes with HA.

In some embodiments, the HA is a hyaluronate salt, including, but not limited to, potassium hyaluronate, magnesium hyaluronate, and calcium hyaluronate.

In some embodiments, the HA may have a small chemical modification. Chemical modifications may be useful for reducing HA degradation, increasing or reducing water solubility, altering the HA rate of diffusion, and/or HA viscosity. Two general approaches are known in the art to chemically modify HA -(1) crosslinking HA using functional chemical reagents and (2) coupling HA using monofunctional reagents. Divinyl sulfone, bisepoxides, formaldehyde, and bishalides are bifunctional reagents which have been used to crosslink HA. Chemically modified HA preparations include, without limitation, aminoethyl methacrylated HA, adipic acid dihydrazide grafted HA, dimethyl ether complexed HA, HA-cysteine ethyl ester, urea-crosslinked HA and N-acetylcysteine HA. Of particular interest are modifications that reduce HA degradation of HA in the eye.

### 2. Pre-Complexation of a Therapeutic Molecule With Hyaluronan (HA) to Form a Conjugate

In some embodiments, the therapeutic molecule is pre-complexed with HA to form a conjugate. The initial concentration of free HABDs comprised in therapeutic molecules can be high at the site of injection, causing detrimental effects, as discussed in Example 5 below. In some instances, these effects may be caused by free HABDs coming into contact with IVT HA at the injection site. Pre-complexation of a HABD with HA diminishes these detrimental effects by giving HABDs time to diffuse from the injection site to the rest of the vitreous. Slower diffusion time and increase in vitreal half-life occurs when HABDs switch from interacting with pre-complexed HA to IVT HA. Thus, in some embodiments, the therapeutic molecule is a conjugate, comprising said therapeutic molecule, and further comprising one or more third components comprising HA.

In some embodiments, the conjugate comprises non-covalent interactions between the therapeutic molecule and the HA. In some embodiments, the conjugate comprises covalent interactions between the therapeutic molecule and the HA.

In some embodiments, the conjugate may be an isolated conjugate, i.e., the conjugate is not within an individual to be treated. In some aspects, a conjugate is purified to greater than 95% or 99% purity as determined by, for example, electrophoresis (e.g., SDS-PAGE, isoelectric focusing (IEF), and capillary electrophoresis) or chromatography (e.g., ion exchange or reverse phase HPLC). For a review of methods for assessing antibody purify, see, e.g., Flatman et al., J Chromatogr B 848:79-87 (2007).

### D. Fusion Proteins

In some embodiments, the first and the second components are proteins, more preferably comprised in a fusion protein; the first and second components are connected via a covalent linker.

Fusion proteins are proteins created by joining of two or more originally separate proteins or peptides. This procedure results in a single polypeptide with functional properties derived from each of the original, separate proteins. The proteins may be fused directly to each other. The proteins may also be fused via a linker, which may increase the likelihood that that the proteins fold independently of each other and behave as expected in each of their native states. Dimeric or multimeric fusion proteins can be manufactured through genetic engineering by fusion to the original proteins of peptide domains that induce protein complexation (such as with antibody domains).

In some embodiments, the second component is directly bound to the first component. This means that the second component directly follows the first component (or vice versa) without further chemical elements (atoms or groups) being present between the two components. In some embodiments, the last amino acid of the first component is immediately adjacent to the first amino acid of the second component. In some embodiments, the last amino acid of the second component is immediately adjacent to the first amino acid of the first component.

In some embodiments, the second component is bound indirectly to the first component via a linker, particularly a peptide linker. In some embodiments, this means that a peptide linker lies in between the first and second components. In some embodiments, a peptide linker lies in between the last amino acid of the first component and the first amino acid of the second component. In some embodiments, a peptide linker lies in between the last amino acid of the second component and the first amino acid of the first component.

In some embodiments, one or two second components are covalently bound to the N-terminus and/or the C-terminus of the first component. In some embodiments, the first component is an antibody or antigen-binding fragment and the one or two second components are covalently bound to a C-terminus of the first component (directly or via a peptide linker. In embodiments where the fusion protein is a Fab-HABD, the HABD is covalently bound to the C-terminus of the Fab.

### 1. Peptide Linkers

In many embodiments, a peptide linker connects the therapeutically active agent (i.e., the first component) and the HABD (i.e., the second component). In some embodiments, the linker comprises at least 4 amino acids. In some embodiments, the linker comprises 4 to 25 amino acids. In some embodiments, the linker comprises 5 to 100 amino acids. In some embodiments, the linker comprises 10 to 50 amino acids. In some embodiments, the linker is no longer than 25 amino acids. In some embodiments, the linker is no longer than 50 amino acids.

In some embodiments, the peptide linker comprises flexible residues like glycine and serine so that the adjacent protein domains are free to move relative to one another. Thus, in some embodiments, the peptide linker is a glycine-serine linker, i.e., a peptide linker consisting of a pattern of glycine and serine residues. In one embodiment said peptide linker is (GxS)ₙ or (GxS)ₙGₘ, wherein G = glycine and S = serine. In these embodiments, x = 3; n = 3, 4, 5 or 6; and m = 0, 1, 2 or 3. In other embodiments, x = 4; n = 2, 3, 4 or 5; and m = 0, 1, 2 or 3. In some embodiments, x = 4 and n = 2 or 3. In some embodiments, x = 4 and n = 2.

In some embodiments the peptide linker is composed of GGGGS (SEQ ID NO: 27) or a multimer thereof, more especially (GGGGS)₃ (SEQ ID NO: 28).

In some embodiments, the peptide linker comprises (GS)ₙ, wherein G is glycine and S is serine. In these embodiments, n = 1, 2, 3, 4, 5, 6, 7, 8, 9, or 10. In some embodiments, n = 10. In some embodiments, the linker is SEQ ID NO: 95.

### E. Certain Embodiments of Therapeutic Molecules and Conjugates

### 1. VEGF

In some embodiments, the therapeutic molecule comprises (1) a first component comprising an anti-VEGF antibody, antibody fragment, antigen-binding fragment, or Fab; and (2) one or two second components, wherein the second components comprise CD44 HA receptor domains, TSG6 domains, and/or a VG1 domains.

In some embodiments, the conjugate comprises (1) a first component comprising an anti-VEGF antibody, antigen-binding fragment, antibody fragment, or Fab; (2) one or two second components; and (3) a HA of molecular weight ranging from 5 kDa to 20 kDa.

### a) G6.31

In some embodiments, the first component is an antibody comprising the G6.31 anti-VEGF Fab. In some embodiments, the first component is an antibody having the VH domain comprised in SEQ ID NO: 17. In some embodiments, the first component is an antibody having the VL domain comprised in SEQ ID NO: 18. In some embodiments, the first component is an antibody comprising the VH domain set forth in SEQ ID NO: 105. In some embodiments, the first component is an antibody comprising the VL domain set forth in SEQ ID NO: 106.

### b) PigFab

In some embodiments, the first component is an antibody comprising PigFab anti-VEGF Fab. In some embodiments, the first component is an antibody having the VH domain comprised in SEQ ID NO: 66. In some embodiments, the first component is an antibody having the VL domain comprised in SEQ ID NO: 65. In some embodiments, the first component is an antibody comprising the VH domain set forth in SEQ ID NO: 97. In some embodiments, the first component is an antibody comprising the VL domain set forth in SEQ ID NO: 98.

### c) Ranibizumab

In some embodiments, the first component is an antibody comprising ranibizumab. In some embodiments, the first component is an antibody having the VH domain comprised in SEQ ID NO: 77. In some embodiments, the first component is an antibody having the VL domain comprised in SEQ ID NO: 76. In some embodiments, the first component is an antibody comprising the VH domain set forth in SEQ ID NO: 114. In some embodiments, the first component is an antibody comprising the VL domain set forth in SEQ ID NO: 115.

### d) CD44

In some embodiments, the one or two second components comprise a CD44 HA receptor domain. In some embodiments, the second component comprises SEQ ID NO: 2.

### e) TSG6

In some embodiments, the one or two second components comprise a TSG6 domain. In some embodiments, the one or two second component comprises SEQ ID NO: 4.

In some embodiments, the therapeutic molecule comprises SEQ ID NO: 17, SEQ ID NO: 18, SEQ ID NO: 19, and/or SEQ ID NO: 20.

### f) VG1

In some embodiments, the one or two second components comprise a VG1 domain. In some embodiments, the one or two second component comprises one or two of the following SEQ ID NOS: 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 87.

In some embodiments, the therapeutic molecule comprises SEQ ID NO: 65, SEQ ID NO: 66, SEQ ID NO: 67, SEQ ID NO: 68, SEQ ID NO: 76 and/or SEQ ID NO: 77.

### g) Cysteine Knot Peptide (CKP)

In some embodiments, the first component, in addition to comprising an anti-VEGF antigen-binding fragment, optionally further comprises a cysteine knot peptide (CKP). In some embodiments, the CKP has at least 90%, 91%, 92%, 93%, 94%, 95%, 96%, 97%, 98%, 99%, or 100% sequence identity with SEQ ID NO: 92.

In some embodiments, the therapeutic molecule has at least 90%, 91%, 92%, 93%, 94%, 95%, 96%, 97%, 98%, 99%, or 100% sequence identity with SEQ ID NO: 93. In some embodiments, the anti-VEGF antigen-binding fragment and has at least 90%, 91%, 92%, 93%, 94%, 95%, 96%, 97%, 98%, 99%, or 100% sequence identity with SEQ ID NO: 94.

In some embodiments, said therapeutic molecules comprising a first component comprising an anti-VEGF antigen-binding fragment and a cysteine knot peptide may further comprise a second component comprising a HABD as discussed in Section II.B above.

In some embodiments, the conjugate comprises (1) a first component comprising an anti-VEGF antigen-binding fragment, (2) one or two second components comprising a HABD, and (3) a HA of molecular weight ranging from 5 kDa to 20 kDa.

### 2. NVS24

In some embodiments, the therapeutic molecule comprises (1) a first component comprising the anti-VEGF antibody, NVS24, and (2) one second component comprising a TSG6 (Lava12) domain.

In some embodiments, the first component comprises the NVS24 antibody. In some embodiments, the first component is an antibody having the VH domain comprised in SEQ ID NO: 21. In some embodiments, the first component is an antibody having the VL domain comprised in SEQ ID NO: 22. In some embodiments, the first component is an antibody comprising the VH domain set forth in SEQ ID NO: 109. In some embodiments, the first component is an antibody comprising the VL domain set forth in SEQ ID NO: 110.

### a) TSG6 (Lava12)

In some embodiments, the second component comprise a TSG6 (Lava12) domain. In some embodiments, the second component comprises SEQ ID NO: 113.

In some embodiments, the therapeutic molecule comprises SEQ ID NO: 21 and/or SEQ ID NO: 22.

### 3. Anti-VEGF and Anti-PDGF Dual-Targeting Antibody (Anti-VP-dutaFab; Anti-VPDF)

In some embodiments, the therapeutic molecule comprises (1) a first component capable of binding VEGF and PDGF (such as a bispecific antibody or a dual-targeting antibody, dutaFab), which is discussed in Section II.A.1.h) above; and (2) one or two second components comprising CD44 HA receptor domains, TSG6 domains, and/or VG1 domains.

In some embodiments, the first component is an antibody having the VH domain comprised in SEQ ID NO: 5. In some embodiments, the first component is an antibody having the VL domain comprised in SEQ ID NO: 6. In some embodiments, the first component is an antibody comprising the VH domain set forth in SEQ ID NO: 99. In some embodiments, the first component is an antibody comprising the VL domain set forth in SEQ ID NO: 100.

### a) CD44

In some embodiments, the one or two second components comprise a CD44 HA receptor domain. In some embodiments, the one or two second components comprise SEQ ID NO: 2.

In some embodiments, the therapeutic molecule comprises SEQ ID NO: 5, SEQ ID NO: 6, SEQ ID NO: 7, and/or SEQ ID NO: 8.

In some embodiments, the one or two second components comprise CD44-ko domain. In some embodiments, the one or two second components comprise the CD44-ko domain set forth in SEQ ID NO: 25 and/or 26.

In some embodiments, the therapeutic molecule comprises SEQ ID NO: 25 and/or 26.

### b) TSG6 (Lava12)

In some embodiments, the second component comprise a TSG6 (Lava12) domain. In some embodiments, the second component comprises SEQ ID NO: 113.

In some embodiments, the therapeutic molecule comprises SEQ ID NO: 23 and/or SEQ ID NO: 24.

### c) VG1

In some embodiments, the one or two second components comprise a VG1 domain. In some embodiments, the one or two second component comprises one or two of the following SEQ ID NOS: 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 87.

In some embodiments, the therapeutic molecule comprises SEQ ID NO: 69, SEQ ID NO: 70, SEQ ID NO: 72, and/or SEQ ID NO: 73.

### 4. RabFab

In some embodiments, the therapeutic molecule comprises (1) a first component comprising a RabFab antibody (discussed in Section II.A.3 above); and (2) one or two second components comprising TSG6 domains and/or VG1 domains.

In some embodiments, the RabFab antibody comprises RabFab VH and VL domains. In some embodiments, the RabFab antibody comprises the VH domain comprised in SEQ ID NO: 13 and the VL domain comprised in SEQ ID NO: 14. In some embodiments, the RabFab antibody comprises the VH domain set forth in SEQ ID NO: 107. In some embodiments, the RabFab antibody comprises the VL domain set forth in SEQ ID NO: 108.

### a) TSG6

In some embodiments, the one or two second components comprise a TSG6 domain. In some embodiments, the one or two second components comprise SEQ ID NO: 4.

In some embodiments, the therapeutic molecule comprises SEQ ID NO: 13, SEQ ID NO: 14, SEQ ID NO: 15, and/or SEQ ID NO: 16.

### b) VG1

In some embodiments, the one or two second components comprise a VG1 domain. In some embodiments, the one or two second component comprises one or two of the following SEQ ID NOS: 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 87.

In some embodiments, the therapeutic molecule comprises SEQ ID NO: 63 and/or SEQ ID NO: 64.

### 5. 20D12v2.3

In some embodiments, the first component is an antibody comprising the anti-complement factor D antibody Fab, 20D12v2.3. In some embodiments, the first component is an antibody having the VH domain comprised in SEQ ID NO: 75. In some embodiments, the first component is an antibody having the VL domain comprised in SEQ ID NO: 74. In some embodiments, the first component is an antibody comprising the VH domain set forth in SEQ ID NO: 111. In some embodiments, the first component is an antibody comprising the VL domain set forth in SEQ ID NO: 112.

### a) VG1

In some embodiments, the one or two second components comprise a VG1 domain. In some embodiments, the one or two second component comprises one or two of the following SEQ ID NOS: 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 87.

In some embodiments, the therapeutic molecule comprises SEQ ID NO: 74 and/or SEQ ID NO: 75.

### 6. HTRA1

In some embodiments, the first component is an antibody comprising an antibody or antibody fragment capable of binding human HtrA1. In some embodiments, the first component is an antibody having the VH domain comprised in SEQ ID NO: 118. In some embodiments, the first component is an antibody having the VL domain comprised in SEQ ID NO: 119. In some embodiments, the first component is an antibody comprising the VH domain set forth in SEQ ID NO: 116. In some embodiments, the first component is an antibody comprising the VL domain set forth in SEQ ID NO: 117.

### a) VG1

In some embodiments, the one or two second components comprise a VG1 domain. In some embodiments, the one or two second component comprises one or two of the following SEQ ID NOS: 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 87.

In some embodiments, the therapeutic molecule comprises SEQ ID NO: 118 and/or SEQ ID NO: 119.

### III. Treatment of Eye Diseases

Materials and methods are useful in the treatment of eye diseases. An eye disease may be characterized by altered or unregulated proliferation and/or invasion of new blood vessels into the structures of ocular tissues such as the retina or cornea. An eye disease may be characterized by atrophy of retinal tissue (photoreceptors and the underlying retinal pigment epithelium (RPE) and choriocapillaris). Non-limiting eye diseases include, for example, age-related macular regeneration (AMD; e.g., wet AMD, dry AMD, intermediate AMD, advanced AMD, and geographic atrophy (GA)), macular degeneration, macular edema, diabetic macular edema (DME) (e.g., focal, non-center DME and diffuse, center-involved DME), retinopathy, diabetic retinopathy (DR) (e.g., proliferative DR (PDR), non-proliferative DR (NPDR), and high-altitude DR), other ischemia-related retinopathies, ROP, retinal vein occlusion (RVO) (e.g., central (CRVO) and branched (BRVO) forms), CNV (e.g., myopic CNV), corneal neovascularization, diseases associated with corneal neovascularization, retinal neovascularization, diseases associated with retinal/choroidal neovascularization, central serous retinopathy (CSR), pathologic myopia, von Hippel-Lindau disease, histoplasmosis of the eye, FEVR, Coats’ disease, Norrie Disease, retinal abnormalities associated with osteoporosis-pseudoglioma syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, including but not limited to CMV retinitis, ocular melanoma, retinal blastoma, conjunctivitis (e.g., infectious conjunctivitis and non-infectious (e.g,. allergic) conjunctivitis), Leber congenital amaurosis (also known as Leber’s congenital amaurosis or LCA), uveitis (including infectious and non-infectious uveitis), choroiditis (e.g., multifocal choroiditis), ocular histoplasmosis, blepharitis, dry eye, traumatic eye injury, Sjögren’s disease, and other ophthalmic diseases wherein the disease or disorder is associated with ocular neovascularization, vascular leakage, and/or retinal edema or retinal atrophy. Additional exemplary eye diseases include diseases caused by the abnormal proliferation of fibrovascular or fibrous tissue, including all forms of proliferative vitreoretinopathy.

Exemplary diseases associated with corneal neovascularization (neovascularization of the iris, neovascularization of the angle, or rubeosis) include, but are not limited to, epidemic keratoconjunctivitis, vitamin A deficiency, contact lens overwear, atopic keratitis, superior limbic keratitis, terygium keratitis sicca, Sjögren’s syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien’s marginal degeneration, marginal keratolysis, rheumatoid arthritis, systemic lupus, polyarteritis, trauma, Wegener’s sarcoidosis, scleritis, Stevens-Johnson syndrome, periphigoid radial keratotomy, and corneal graph rejection.

Exemplary eye diseases associated with choroidal neovascularization and defects in the retina vasculature, including increased vascular leak, aneurisms and capillary drop-out include, but are not limited to, diabetic retinopathy, macular degeneration, sickle cell anemia, sarcoid, syphilis, pseudoxanthoma elasticum, Paget’s disease, vein occlusion, artery occlusion, carotid obstructive disease, chronic uveitis/vitritis, mycobacterial infections, Lyme’s disease, systemic lupus erythematosis, retinopathy of prematurity, retina edema (including macular edema), Eales disease, Behcet’s disease, infections causing retinitis or choroiditis (e.g., multifocal choroidits), presumed ocular histoplasmosis, Best’s disease (vitelliform macular degeneration), myopia, optic pits, pars planitis, retinal detachment (e.g., chronic retinal detachment), hyperviscosity syndromes, toxoplasmosis, trauma, and post-laser complications.

Exemplary eye diseases associated with atrophy of retinal tissues (photoreceptors and the underlying RPE) include, but are not limited to, atrophic or nonexudative AMD (e.g., geographic atrophy or advanced dry AMD), macular atrophy (e.g., atrophy associated with neovascularization and/or geographic atrophy), diabetic retinopathy, Stargardt’s disease, Sorsby Fundus Dystrophy, retinoschisis (abnormal splitting of the retina neurosensory layers) and retinitis pigmentosa.

In certain embodiments according to (or as applied to) any of the embodiments above, the eye disease is an intraocular neovascular disease selected from the group consisting of proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, retinal vein occlusion (RVO), including CRVO and BRVO, corneal neovascularization, retinal neovascularization, and retinopathy of prematurity (ROP). In a preferred embodiment of the present invention, the eye disease is age-related macular degeneration (AMD), particularly wet AMD or neovasular AMD, diabetic macular edema (DME), diabetic retinopathy (DR), particularly proliferative DR or non-proliferative DR, retinal vein occlusion (RVO) or geographic atrophy (GA).

The therapeutic molecules, conjugates, and compositions disclosed herein may be used as medicament for treating an eye disease in a mammalian subject. Examples of mammals include, but are not limited to, domesticated animals (e.g., cows, sheep, cats, dogs, and horses), primates (e.g., humans and non-human primates such as monkeys), rabbits, and rodents (e.g., mice and rats). Preferably, the subject is a human. In some embodiments, the therapeutic target of the therapeutic molecules, conjugates, and compositions is a target in the human eye.

### IV. Methods of Treatment

Provided herein are methods for treating an eye disease comprising delivery of a therapeutic molecule, conjugate, or composition to a tissue in a patient. In many embodiments, the methods comprise administering the therapeutic molecule such that the therapeutic molecule may provide long-acting delivery of the therapeutically active agent to the target tissue. In many embodiments, the target tissue is in the eye.

### A. Methods of Administration

The therapeutic molecules, conjugates, or compositions may be administered in any effective, convenient manner including, for instance, administration by topical, oral, intravenous, intraperitoneal, intramuscular, subcutaneous, intranasal, intratracheal or intradermal routes, among others. It is preferred that the composition be suitable for administration to the eye, more specifically, the composition may be suitable for IVT administration. Accordingly, in a preferred embodiment, the composition is formulated for intraocular delivery, particularly IVT injection. In therapy or as a prophylactic, the therapeutic molecules, conjugates, or compositions may be administered to an individual as an injectable composition, for example, as a sterile aqueous dispersion.

Without being bound to this theory, it is assumed that injecting a conjugate could facilitate diffusion of HA from the pre-complexed HABD before interaction with IVT HA. As the dissociation is slow, the concentrations of free HABD in the vitreous are low. The lower concentrations of free HABD present in the vitreous may be less harmful for the eye than the therapeutic molecules that are not pre-complexed with HA.

In some embodiments, the administering step is a single injection. In some embodiments, the administering step comprises more than a single injection.

### B. Compositions

Compositions for use as a medicament, particularly in the treatment of an eye disease, are provided herein. Compositions may be referred to as pharmaceutical compositions as they are intended for use in the pharmaceutical field or as a pharmaceutic and refers to a preparation which is in such form as to permit the biological activity of an active ingredient contained therein to be effective, and which contains no additional components which are unacceptably toxic to a subject to which the pharmaceutical composition would be administered.

In some embodiments, the composition comprises a therapeutic molecule. In some embodiments, the composition comprises a conjugate.

In some embodiments, the composition optionally comprises a pharmaceutically acceptable excipient, diluent, or carrier, such as buffer substances, stabilizers, preservatives, or further ingredients, especially ingredients commonly known in connection with pharmaceutical compositions.

In general, the nature of optional or additional ingredients will depend on the particular form of composition and the mode of administration being employed. Pharmaceutically acceptable carriers can enhance or stabilize the composition, or can be used to facilitate preparation of the composition. Such carriers may include, but are not limited to, saline, buffered saline, dextrose, water, glycerol, solvents, dispersion media, coatings, antibacterial and antifungal agents, isotonic and absorption delaying agents, and the like that are physiologically compatible as well as combinations thereof. The compositions can additionally contain one or more other therapeutic agents, particularly those suitable for treating or preventing, for example, conditions or disorders associated with an eye disease such as retinal vascular disease. The formulation should suit the mode of administration. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically neutral carriers, pharmaceutical compositions to be administered can contain minor amounts of non-toxic auxiliary substances, such as wetting or emulsifying agents, preservatives, and pH buffering agents and the like.

The composition may comprise a stabilizer. The term “stabilizer” refers to a substance which protects the composition from adverse conditions, such as those which occur during heating or freezing, and/or prolongs the stability or shelf-life of the conjugate of the invention in a condition or state. Examples of stabilizers include, but are not limited to, sugars, such as sucrose, lactose and mannose; sugar alcohols, such as mannitol; amino acids, such as glycine or glutamic acid; and proteins, such as human serum albumin or gelatin.

### C. Effective Dose

Typically, a therapeutically effective dose or efficacious dose of the therapeutic molecule or conjugate is employed in the pharmaceutical compositions of the disclosure. The therapeutic molecules and conjugates are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients (i.e., the therapeutic molecules and conjugates) in the pharmaceutical compositions can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. Dosage level may be selected and/or adjusted to achieve a therapeutic response as determined using one or more of the ocular/visual assessments described herein. A physician or veterinarian can start doses of the therapeutic molecule of conjugate employed in the pharmaceutical composition at levels lower than that required to achieve the desired therapeutic effect and gradually increase the dosage until the desired effect is achieved. In general, effective doses of the compositions for the treatment of an eye disease described herein vary depending upon many different factors, including means of administration, target site, physiological state of the patient, whether the patient is human or an animal, other medications administered, and whether treatment is prophylactic or therapeutic.

Treatment dosages need to be titrated to optimize safety and efficacy. Dosage for IVT administration with a conjugate of the invention may range from 0.1 mg/eye to 10 mg/eye per injection. A single dose per eye may be carried out in 1 or more injections per eye. For example, a single dose of 20 mg/eye may be delivered in 2 injections of 10 mg each, resulting in a total dose of 20 mg. The volume per injection may be between 10 microliters and 50 microliters, while the volume per dose may be between 10 microliters and 100 microliters. The US Food and Drug Administration (FDA)-approved doses and regimes suitable for use with Lucentis are considered. Other doses and regimes suitable for use with anti-VEGF antibodies or antigen-binding fragments are described in US 2012/0014958 and is incorporated by reference in its entirety.

A composition may be administered on multiple occasions. Intervals between single dosages can be weekly, monthly or yearly. Intervals can also be irregular as indicated by the need for retreatment in the patient, based for example on visual acuity or macular edema. In addition, alternative dosing intervals can be determined by a physician and administered monthly or as necessary to be efficacious. Efficacy is based on condition of the eye as well as the kind and severity of the eye disease, e.g., characterized by the lesion growth, rate of anti-VEGF rescue, retinal thickness as determined by Optical Coherence Tomography (OCT), and visual acuity. Dosage and frequency may vary depending on the half-life of the conjugate of the invention in the patient and levels of the therapeutic target (e.g., VEGF, C5, EPO, Factor P, etc.). However, in a preferred embodiment of the present invention, the composition is to be administered at most every three months, particularly at most every four months, more particularly every six months. This reflects the increased half-life (and thus the extended duration of efficacy) of the first component in the conjugate as compared to the respective unbound (free) first component. In accordance with this, the elimination half-life of the first component in the conjugate is extended at least 3-fold, at least 4-fold or at least 5-fold as compared to the unconjugated first component. Relative increases in elimination half-life for the first component in the conjugate compared to the free first component can be determined by administering the molecules by IVT injection and measuring the concentrations remaining at various time points using analytical methods known in the art, for example ELISA, mass spectrometry, western blot, radio-immunoassay, or fluorescent labeling. Blood concentrations can also be measured and used to calculate the rate of clearance from the eye as described (Xu L et al., invest Ophthalmol Vis ScL, 54(3):1816-24 (2013)) in general, molecules (for example, antibodies or fragments) as part of the conjugate show longer ocular half-life than that of free molecules. For example, a conjugate in the eye can have a 25% increase (e.g., from 5 to 6.25 days) in half-life compared to the free first component, a 50% increase (e.g., from 5 to 7.5 days) in half-life compared to the free first component, a 75% increase (e.g., from 5 to 8.75 days) in half-life compared to the free first component, or a 100% increase (e.g., from 5 to 10 days) in half-life compared to the free first component, in certain aspects, it is contemplated that half-life of the conjugate may increase more than 100% in half-life compared to the free first component (e.g., from 5 to 15, 20 or 30 days; from 1 week to 3 weeks, 4 weeks or more; etc.).

### D. Combination Therapies

Combination therapies encompass combined administration (where two or more therapeutic agents are included in the same or separate formulations), and separate administration, in which case, administration of the therapeutic molecules and conjugates can occur prior to, simultaneously, and/or following, administration of the additional therapeutic agent or agents. In certain embodiments, a therapeutic molecule, conjugate, or composition is administered simultaneously with additional compounds. In certain embodiments, the therapeutic molecule, conjugate, or composition is administered before or after the additional compounds. In some embodiments, administration of the therapeutic molecule, conjugate, or composition and administration of an additional therapeutic agent occur within about one, two, three, four, or five months, or within about one, two or three weeks, or within about one, two, three, four, five, or six days, of each other.

Any suitable therapeutic agent for the treatment of an eye disease can be used as said additional compound, particularly an agent for treatment of an eye disease. Eye diseases are discussed in Section III above. Further, any molecule discussed in Section II.A above as a component of a therapeutic molecule may also be used as an additional compound used in combination therapy.

In some embodiments, the additional compound is an anti-angiogenic agent discussed in Section II.A.1.h) above and in Carmeliet et al., Nature 407:249-257 (2000). Other suitable anti-angiogenic agents include corticosteroids, angiostatic steroids, anecortave acetate, angiostatin, endostatin, tyrosine kinase inhibitors, matrix metalloproteinase (MMP) inhibitors, insulin-like growth factor-binding protein 3 (IGFBP3), stromal derived factor (SDF-1) antagonists (e.g., anti-SDF-1 antibodies), pigment epithelium-derived factor (PEDF), gamma-secretase, Delta-like ligand 4, integrin antagonists, hypoxia-inducible factor (HIF)-1α antagonists, protein kinase CK2 antagonists, agents that inhibit stem cell (e.g., endothelial progenitor cell) homing to the site of neovascularization (e.g., an anti-vascular endothelial cadherin (CD-144) antibody and/or an anti-SDF-1 antibody), and combinations thereof.

The therapeutic molecule, conjugate, or composition may also be administered in combination with a therapy or surgical procedure for treatment of an eye disease (e.g., AMD, DME, DR, RVO, or GA), including, for example, laser photocoagulation (e.g., panretinal photocoagulation (PRP)), drusen lasering, macular hole surgery, macular translocation surgery, implantable miniature telescopes, PHI-motion angiography (also known as micro-laser therapy and feeder vessel treatment), proton beam therapy, microstimulation therapy, retinal detachment and vitreous surgery, scleral buckle, submacular surgery, transpupillary thermotherapy, photosystem I therapy, use of RNA interference (RNAi), extracorporeal rheopheresis (also known as membrane differential filtration and rheotherapy), microchip implantation, stem cell therapy, gene replacement therapy, ribozyme gene therapy (including gene therapy for hypoxia response element, Oxford Biomedica; Lentipak, Genetix; and PDEF gene therapy, GenVec), photoreceptor/retinal cells transplantation (including transplantable retinal epithelial cells, Diacrin, Inc.; retinal cell transplant, e.g., Astellas Pharma US, Inc., ReNeuron, CHA Biotech), acupuncture, and combinations thereof.

The therapeutic molecule, conjugate, or composition may also be administered in combination with a visual cycle modifier (e.g., emixustat hydrochloride); squalamine (e.g., OHR-102; Ohr Pharmaceutical); vitamin and mineral supplements (e.g., those described in the Age-Related Eye Disease Study 1 (AREDS1; zinc and/or antioxidants) and Study 2 (AREDS2; zinc, antioxidants, lutein, zeaxanthin, and/or omega-3 fatty acids)); a cell-based therapy, for example, NT-501 (Renexus); PH-05206388 (Pfizer), huCNS-SC cell transplantation (StemCells), CNTO-2476 (umbilical cord stem cell line; Janssen), OpRegen (suspension of RPE cells; Cell Cure Neurosciences), or MA09-hRPE cell transplantation (Ocata Therapeutics).

In some embodiments, the additional therapeutic agent is an AMD therapeutic agent. For example, the anti-PDGFR antibody REGN2176-3 can be co-formulated with aflibercept (EYLEA®). In some instances, such a co-formulation can be administered in combination with a therapeutic molecule, conjugate or composition.

In some embodiments, the additional compound comprises a lentiviral vector expressing endostatin and angiostatin (e.g., RetinoStat).

In certain embodiments, the additional compound binds to a second biological molecule selected from the group consisting of IL-1β; IL-6; IL-6R; IL-13; IL-13R; PDGF; angiopoietin; Ang2; Tie2; S1P; integrins αvβ3, αvβ5, and α5β1; betacellulin; apelin/APJ; erythropoietin; complement factor D; TNFα; HtrA1; a VEGF receptor; ST-2 receptor; and proteins genetically linked to AMD risk, such as complement pathway components C2, factor B, factor H, CFHR3, C3b, C5, C5a, and C3a; HtrA1; ARMS2; TIMP3; HLA; interleukin-8 (IL-8); CX3CR1; TLR3; TLR4; CETP; LIPC; COL10A1; and TNFRSF10A. In certain embodiments, the additional compound is an antibody or antigen-binding fragment thereof, including examples of antibodies and antigen-binding fragments discussed in Section II.A.3 above.

### E. Target Tissue

In some embodiments, the target tissue comprises the eye, brain, bone, and/or tumor. In some embodiments, the tissue comprises the retina. In some embodiments, the therapeutic molecule, conjugate, or composition is injected into the eye, brain, bone, or tumor. In some embodiments, the therapeutic molecule, conjugate, or composition is injected into vitreous humor, cerebrospinal fluid, or synovial fluid. In some embodiments, the therapeutic molecule, conjugate, or composition is injected subcutaneously.

In some embodiments, the therapeutic molecule, conjugate, or composition provides improved compatibility, longer residence time, and/or longer half-life with respect to the injection site in comparison to unmodified therapeutically active ahgent. In some embodiments, the therapeutic molecule, conjugate, or composition may further provide improved duration of pharmacological effect at the target tissue in comparison to unmodified therapeutically active agent.

In some embodiments, the therapeutic molecule, conjugate, or composition provides improved vitreous compatibility, longer vitreous residence time, longer vitreous half-life, and/or improved duration of pharmacological effect in comparison to unmodified therapeutically active agent. In some embodiments, the therapeutic molecule, conjugate, or composition provides improved compatibility, longer residence time, longer half-life, and/or improved duration of pharmacological effect in the brain, synovial joints, or tumors, in comparison to unmodified therapeutically active agent.

### F. Binding the Therapeutic Molecule to HA

In some embodiments, the method comprises binding the therapeutic molecule to HA (i.e., pre-complexing the therapeutic molecule with HA to form a conjugate) before the administering step. In these embodiments, pre-complexing allows for the therapeutic molecule to bind to HA. In some of these embodiments, the HA is bound to the therapeutic molecule’s HABD. Examples of HABDs are discussed in Section II.B above.

In some embodiments, the method comprises mixing a first solution comprising the therapeutic molecule and a second solution comprising the HA. In some embodiments, the mixing comprises a vessel. Examples of a vessel include a vial, a single-compartment syringe, and a two-compartment syringe. In some embodiments, the mixing produces a therapeutic molecule bound to HA that is ready for administering to a subject.

In some embodiments, the HA ranges in size from 400 Da to 200 kDa. In some embodiments, the HA is at least 5 kDa. In some embodiments, the HA is 10 kDa. In some embodiments, the HA size/amount allows for a molar excess of HA to the number of HA binding sites present in the bound or pre-complexation mixture. In some embodiments, the HA size/amount provides a molar excess of binding equivalents to the HABD. In some embodiments, the HA size/amount allows for a ratio of HA to therapeutic molecule that ranges from 1.5:1 to 1:1.

### EXAMPLES

The following are examples of methods and compositions of the invention. It is understood that various other embodiments may be practiced, given the general description provided above.

The following examples discuss fusion proteins comprising Fab fragments or peptides, and hyaluronan-binding domains, i.e., Fab-HABDs. Examples 1-7 relate to CD44 and/or TSG6 HABDs. Examples 8-18 relate to VG1 HABDs.

### Example 1. Generation of Fab-Hyaluronan-Binding Domain Fusion Proteins (Fab-HABDs) and Complexation With HA

Ten fusion proteins of Fab fragments and hyaluronan-binding domains (named Fab-HABDs hereafter) were generated (Table 2). The Fab-HABDs were created by recombinant fusion of one HABD to the C-terminus of the heavy chain of the Fab fragment via Gly-Ser-containing linker sequences (herein termed “1x versions”). In some cases, an additional HABD was fused to the C-terminus of the light chain of the Fab fragment (herein termed “2x versions”).

Fab fragments specifically binding to VEGF and PDGF (termed “VPDF”), Digoxigenin (termed “Dig”), and VEGF (clone “G6.31”) were used to generate the Fab-HABDs.

HABDs were derived from CD44 (SEQ ID NO: 2) or TSG6 (SEQ ID NO: 4).

The Dig antibody was covalently linked to one or two CD44 HA receptor domains and used as non-binding control molecules (SEQ ID NOS: 9-12).

TABLE 2

Amino acid sequences of Fab-HABDs used in the examples

| | | |
|---|---|---|
| Name | HC | LC |
| VPDF-1xCD44 | SEQ ID NO: 5 | SEQ ID NO: 6 |
| VPDF-2xCD44 | SEQ ID NO: 7 | SEQ ID NO: 8 |
| Dig-1xCD44 | SEQ ID NO: 9 | SEQ ID NO: 10 |
| Dig-2xCD44 | SEQ ID NO: 11 | SEQ ID NO: 12 |
| RabFab-1xTSG6 | SEQ ID NO: 13 | SEQ ID NO: 14 |
| RabFab-2xTSG6 | SEQ ID NO: 15 | SEQ ID NO: 16 |
| G6.31-1xTSG6 | SEQ ID NO: 17 | SEQ ID NO: 18 |
| G6.31-2xTSG6 | SEQ ID NO: 19 | SEQ ID NO: 20 |
| NVS24-1xTSG6 (Lava12) | SEQ ID NO: 21 | SEQ ID NO: 22 |
| VPDF-1xTSG6 (Lava12) | SEQ ID NO: 23 | SEQ ID NO: 24 |
| VPDF-2xCD44-ko (control) | SEQ ID NO: 25 | SEQ ID NO: 26 |

### A. Materials and Methods

### 1. Protein Expression

Expression plasmids for the various Fab-HABDs were generated by restriction cloning or gene synthesis using standard molecular biology techniques. Separate expression vectors were generated for each polypeptide chain. Expression was performed in HEK293 cells (ThermoFisher) and expression plasmids were mixed in a 1:1 ratio.

In some instances, TSG6 was expressed in E. coli.

In some instances, RabFab-1xTSG6 and RabFab-2xTSG6 were produced by secretion from stably transfected Chinese hamster ovary (CHO) cells.

### 2. Protein Purification

Supernatants were harvested by centrifugation at 4,000 rpm, 4° C., for 20 minutes. Thereafter cell-free-supernatant was filtered through a 0.22 µm bottle-top-filter and stored in a freezer (-20° C.).

Fab-HABDs were purified from cell culture supernatants by affinity chromatography using anti-Ckappa and anti-CH1 resin together with size exclusion chromatography (SEC).

Briefly, sterile filtered cell culture supernatants were captured on KappaSelect resin (GE Healthcare) equilibrated with 1 × PBS buffer (10 mM Na2HPO4, 1 mM KH₂PO₄, 137 mM NaCl and 2.7 mM KC1, pH 7.4), washed with equilibration buffer and eluted with 100 mM sodium citrate at pH 2.8. The eluted antibody fractions were pooled and the pH was adjusted to 7.5. Protein was then captured on CaptureSelect IgG-CH1 resin (Life Technologies) equilibrated with 1 × PBS buffer (10 mM Na₂HPO₄, 1 mM KH₂PO₄, 137 mM NaCl and 2.7 mM KC1, pH 7.4), washed with equilibration buffer and eluted with 100 mM sodium citrate at pH 2.8. Concentrations of protein samples were determined on a Nanodrop 800 Spectrophotometer (Thermo Scientific) at 280 nm.

Analytical SEC was carried out via a HiLoad 16/60 Superdex 200 prep grade column (GE Healthcare) using a 20 mM histidine, 140 mM NaCl, pH 6.0 running buffer at a flow rate of 1.5 mL/min.

Antibody-containing pooled fractions from size exclusion chromatography were frozen at -80° C. and stored for further use.

In some instances, TSG6 was purified from E. coli. Briefly, E. coli cells were extracted using a buffer consisting of 7 M guanidine-HC1, 50 mM Tris-HCl, 100 mM sodium tetrathionate, and 20 mM sodium sulfite. After homogenization using a Polytron® homogenizer, centrifugation and filtration of the supernatant, the his-tagged protein was captured on a Ni-NTA column (GE Healthcare) equilibrated with 6 M guanidine-HCl, 25 mM Tris-HCl, pH 8.6. The column was washed with 25 mM Tris-HCl pH 8.6, 0.1% Triton X-114 and eluted with buffer containing 250 mM imidazole. TSG6 eluted from the column was refolded by dilution to 1.5 mg/mL followed by overnight dialysis at a temperature of 4° C. versus a solution of 0.5 M guanidine-HCl, 0.5 M 1-arginine, 1 mM reduced glutathione (GSH) and 1 mM oxidized glutathione (GSSG). After buffer exchange into 25 mM sodium acetate, pH 5.0, the refolded material was purified by cation exchange chromatography on SP-Sepharose™ (GE Healthcare).

In some instances, RabFab-1xTSG6 and RabFab-2xTSG6 were secreted by stably transfected Chinese hamster ovary (CHO) cells and purified from cell culture media. These proteins did not require refolding. RabFab-1xTSG6 has this Fab fused to TSG- via a gly-gly-gly-gly-ser linker; the HABD is on the C-terminus of HC. RabFab-2xTSG6 has this Fab fused to TSG6 via a gly-gly-gly-gly-ser linker; one HABD is on the C-terminus of HC while another is on the C-terminus of LC. Both proteins have a His-tag at the C-terminus of the heavy chain for use in purification. These Fab-HABDs were purified from CHO supernatants using 3 column chromatography steps consisting of (1) capture on an antigen-affinity column as described in Shatz, W. et al., Mol. Pharm., 13(9):2996-3003 (2016), (2) isolation of His-tagged material on a Nickel-NTA column followed by (3) cation exchange chromatography on SP-Sepharose.

### 3. Complexation With Hyaluronan (HA)

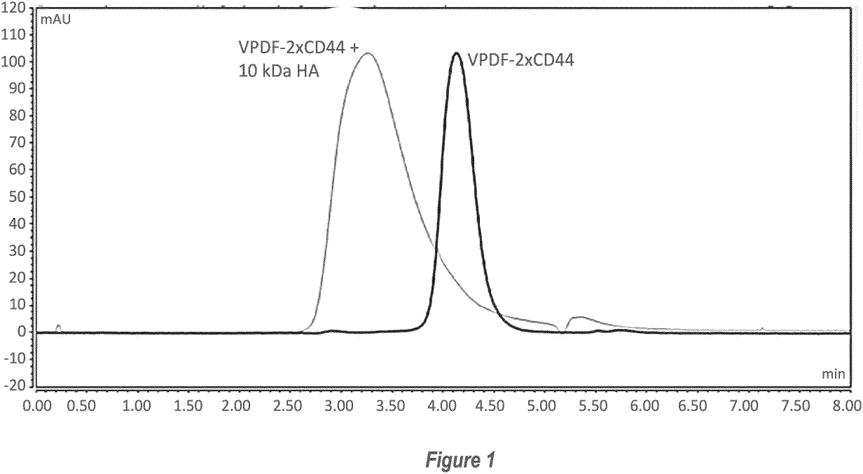

Fab-HABDs were mixed 1:1 (w/w) with 10 kDa Sodium Hyaluronate (Lifecore, Biomedical) for formation of Fab-HABD-HA conjugates (hereafter named Fab-HABD-HAs). After mixing, the conjugate was concentrated and rebuffered with Amicon Ultra 10 kDa cut off (Millipore). The final formulation was 20 mM histidine pH 6.0, 260 mM Sucrose, 140 mM NaCl, 0.02% Tween 20. Finally, the conjugate was filtered through a 0.22 µm filter (Ultrafree-MC, Centrifugal Units 0.22 µm, GV Durapore). Formation of Protein-HA conjugates was monitored by a shift in SEC to shorter retention times in comparison to the respective Fab-HABD (see FIG. 1).

### Example 2. Molecular Properties of Fab-HABDs

### Example 2.1. Interaction With HA

### A. Materials and Methods

The ability of the HABD of Fab-HABDs to bind to HA was examined. Binding of Fab-CD44 and Fab-TSG6 Fab-HABDs to HA was tested by SPR using a Biacore T200 instrument (GE Healthcare) (Table 3). Briefly, the Fab-CD44 Fab-HABDs were injected for 80 sec or 120 sec onto a HA coated chip (SCBS HY, Xantect Bioanalytics GmbH, Germany) with concentrations ranging from 3.7 to 300 nM each. For some experiments, HA-coated chips were prepared by indirect coupling of biotin-HA (Sigma-Aldrich, St. Louis, Missouri U.S.) onto a Series S Sensor SA Chip coated with streptavidin (GE Healthcare). The dissociation phase was monitored for 600 sec. Subsequently, the surface was regenerated by injecting 10 mM Glycine pH 1.5 for 60 sec or 3 M MgCl₂ for 30 sec. Bulk refractive index differences were corrected by subtracting the response obtained from buffer injections. All experiments were performed at 25° C. using PBS-T (10 mM Na2HPO4, 1 mM KH2PO4, 137 mM NaCl, 2.7 mM KCl pH 7,4, 0.05% Tween-20). The derived curves were fitted to a 1:1 Langmuir binding model using the BIAevaluation software. All experiments were performed at 25° C. using PBS-T (10 mM Na₂HPO₄, 1 mM KH₂PO₄, 137 mM NaCl, 2.7 mM KCl pH 7,4, 0.05% Tween-20).

In addition, interaction of VDPF-2xCD44 with HA was tested by isothermal titration calorimetry (ITC). Briefly, Fab-CD44 fusions were dialyzed against PBS (10 mM Na₂HPO₄, 1 mM KH₂PO₄, 137 mM NaCl, 2.7 mM KCl pH 7,4). After dialysis, the remaining buffer was used to dissolve the HA so all molecules were in exactly the same buffer conditions to avoid any buffer related mismatch. The HA molecules were loaded into the sample cell at a concentration of 10 µM (10 kDa HA) or 2 µM (50 kDa HA), respectively. The reference cell was loaded with deionized water. The syringe was filled with the Fab-CD44 fusion at a concentration of 150 µM. The titration experiments were performed at 25° C. The affinity constant K as well as the stoichiometry N was calculated using the one set of sites model in Origin 7.0 (OriginLab Corporation).

Similarly, ITC was used to measure the interaction of TSG6 with 10 kDa HA (Table 4), except that for these experiments the TSG6 (20 µM) was placed in the calorimeter cell and titrated with HA (50 µM) in the syringe. Solutions containing PBS were prepared as described above and the temperature of the measurements was 25° C. or 37° C. These measurements were performed on an Auto PEAQ ITC instrument (Malvern Instruments). Data analysis was as described in the preceding paragraph except that N was held fixed at 1.0 and the HA concentration and affinity constant K were variable parameters.

### B. Results

K_{D} by SPR for HA binding by Fab-CD44s and Fab-TSG6s are shown in Table 3.

TABLE 3

Interaction between Fab-CD44 and Fab-TSG6 molecules with HA (SPR)

| SEQ ID NOS | Molecule | K_{D} [µM] |
|---|---|---|
| 5, 6 | VPDF-1xCD44 | >10 |
| 7, 8 | VPDF-2xCD44 | ~ 0.6 |
| 25, 26 | VPDF-2xCD44-ko | No binding |
| 11, 12 | Dig-2xCD44 | ~ 0.6 |
| 17, 18 | G6.31-1xTSG6 | 1.5 |
| 19, 20 | G6.31-2xTSG6 | 0.01 |
| 23, 24 | VPDF-1xTSG6 (Lava12) | 1.5 |
| 4 | TSG6 | 0.9 |
| 13, 14 | RabFab-1xTSG6 | 1.6 |
| 15, 16 | RabFab-2xTSG6 | 0.09 |
| 21, 22 | NVS24-1xTSG6 (Lava12) | 1.2 |

Strength of the interaction is determined by the HABD sequence as well as by the avidity of the interaction (i.e., 2x-versions show higher functional affinity via avid binding to HA).

ITC analysis (Table 4) yielded the HA affinity shown below with binding site concentration calculated as 400-745 µM indicating an estimated stoichiometry of 8-15 TSG6 molecules per 10 kDa HA chain. A similar experiment using 50 µM VPDF-2xCD44 in the cell and 150 µM 10 kDa HA in the syringe yielded a K_{D} of 25 µM and apparent stoichiometry of 4.5 VPDF-2xCD44 per 10 kDa HA chain. The weaker HA-binding affinity of CD44 required that higher concentrations be used for ITC experiments. As might be expected from the bivalent HA-binding nature of the 2xCD44 fusion, and higher molecular weight of CD44 compared to TSG6, the binding stoichiometry for 10 kDa is 2-3-fold greater for 1xTSG6 relative to 2xCD44. Strength of the interaction is determined by the HABD sequence as well as by the aviditiy of the interaction (i.e., 2x-versions show higher functional affinity via avid binding to HA).

TABLE 4

Interaction between Fab-CD44 and Fab-TSG6 molecules with HA (ITC

| SEQ ID NOS | Molecule | HA | K_{D} [µM] | Estimated stoichiometry |
|---|---|---|---|---|
| 7, 8 | VPDF-2xCD44 | 10 kDa | 2.2-5 | 1.5 |
| 7, 8 | VPDF-2xCD44 | 50 kDa | 0.7 | 5 |
| 4 | TSG6 | 10 kDa | 9.2 (Temp. = 25° C.) | 14.9 |
| 13, 14 | RabFab-1xTSG6 | 10 kDa | 17.7 (Temp. = 25° C.) | 12.3 |
| 13, 14 | RabFab-1xTSG6 | 10 kDa | 7.9 (Temp. = 37° C.) | 8.2 |
| 7, 8 | VPDF-2xCD44 | 10 kDa | 25.0 (Temp.= 37° C.) | 4.5 |

In terms of stoichiometry of the interaction, we found that on average, 1.5 VPDF-2xCD44 could be bound per 10 kDa HA molecule, whereas 5 VPDF-2xCD44 could be bound per 50 kDa HA molecule.

According to SPR measurements, VPDF-2xCD44 was capable of binding both VEGF and PDGF ligands simultaneously. The binding of VEGF and PDGF to VPDF-2xCD44 were compared to their binding to unmodified VPDF. Briefly, PDGF was coupled to a Series S Sensor Chip CM5 (GE Healthcare) using standard coupling chemistry resulting a surface density of appr. 4000 resonance units (RU). After injecting the VPDF-2xCD44 fusions as well as an unmodified VPDF control at concentration of 3 µg/mL each, VEGF was injected at a concentration of 5 µg/mL to demonstrate simultaneous binding of the Fabs to both ligands PDGF and VEGF. Subsequently, the surface was regenerated by injecting 10 mM Glycine pH 2.0 for 60 sec. SPR measurements confirmed that fusion of an HABD to the C-terminus of the VPDF Fab fragment heavy chain does not disturb interactions of the ligands with the target proteins.

### Example 2.2. Stability of Fab-HABDs

Use of Fab-HABDs for long-acting delivery in the eye requires protein stability at body temperature on a months-long scale. A prerequisite for this is thermal stability of Fab-HABDs that is higher than 37° C.

### A. Materials and Methods

Thermal stability of VPDF-2xCD44 and TSG6 were tested by static light scattering and protein autofluorescence. Samples were diluted to approximately 1 mg/mL and subjected to a temperature ramp from 25° C. to 80° C. with a heat rate of 0.1° C./min using an Optim instrument (Avacta Inc.). Light scattering and fluorescence data were recorded during this process upon irradiation with a 266 nm laser. An aggregation onset, defined as the temperature at which the scattering intensity increases, of approximately 75° C. was determined. Simultaneously, fluorescence emission spectra were recorded.

For VPDF-CD44, two transitions were measured at approximately 56° C. and 79° C. when the barycentric mean of the fluorescence spectrum was plotted vs. temperature. These transitions indicate denaturation of the protein, likely of the Fab and CD44 domains. Any scattering or spectral change related to thermal unfolding is thus >>37° C. which indicates good stability of this Fab-HABD.

### B. Results

Two transitions were measured for VPDF-2xCD44 at 56° C. and at 79° C. These two Tₘs indicate transitions in the denaturation of VPDF-2xCD44, with the CD44 domains denaturing at 56° C. and the Fab denaturing at 79° C.

For TSG6, the observed T_{m onset} was measured to be 35° C. with a measured Tₘ of 43° C.

### Example 3. In Vivo Efficacy in Rat Laser Choroidal Neovascularization (CNV)

### A. Materials and Methods

Fab-HABDs were studied in an in vivo rat model of laser-induced choroidal neovascularization (rat laser CNV) to test the following assumptions: (1) Fab-HABDs are efficacious in vivo (i.e., Fab-HABDs can inhibit neovascularization) despite binding to IVT HA; and (2) Fab-HABDs have a longer-lasting in vivo efficacy in comparison to the respective unmodified Fab fragment.

For this, rats received an IVT injection of a protein formulation either one week or three weeks before undergoing laser injury (6 laser burns per eye). One week after setting the laser injury, lesions were analyzed for vascular growth with a fluorescence angiography (FA) imaging.

Fab-HABDs were compared to the respective unmodified Fab fragments. For detection of long-lasting efficacy of Fab-HABDs, the dose of unmodified Fab was titrated to a “minimal effect dose” (i.e., only low detectable inhibition of neovascularization in comparison to vehicle within the duration of the rat model) to show longer lasting efficacy for an Fab-HABD at the same dose and duration of the model.

### B. Results

All tested Fab-HABDs showed inhibition of neovascularization in vivo (Table 5). This shows that the Fab-HABDs reach the relevant tissues to exert a pharmacologic effect although they were bound to HA in the vitreous.

TABLE 5

In vivo efficacy in rat laser CNV

| SEQ ID NOS | Molecule | Dose | % inhibition CNV, 1 week model | % inhibition CNV, 3 week model |
|---|---|---|---|---|
| n/a | vehicle | - | 0 | 0 |
| 101, 102 | VPDF (unmodified) | 0.01 µg | 44 | 34 |
| 5, 6 | VPDF-1xCD44 | 0.01 µg | 84 | 82 |
| 7, 8 | VPDF-2xCD44 + 10 kDa HA* | 0.01 µg | 72 | 81 |
| 23, 24 | VPDF-1xTSG6 (Lava12) | 0.01 µg | N/A | 69 |

*VPDF-2xCD44 was tested pre-complexed to 10 kDa HA

All tested Fab-HABDs showed a longer duration of the pharmacologic effect in comparison to the respective unmodified Fab fragment at the same dose within the same model setup (Table 5). This shows that the ability to bind to IVT HA can prolong the pharmacologic effect in vivo.

The resolution of the in vivo model did not allow for differentiation of durability of efficacy for different molecules, despite significant differences in affinity towards HA. At the low, non-therapeutic doses that were applied in the model, no tolerability issues were detected. All eyes of rats that received doses of Fab-HABDs were completely normal with no signs of disturbance and comparable to eyes that received buffer only during the in-life phase.

### Example 4. Rabbit Pharmacokinetic (PK) Studies With RabFab, RabFab-1xTSG6 and RabFab-2xTSG6

### A. Materials and Methods

Proteins for animal studies were formulated in either 20 mM Histidine Acetate, 150 mM NaCl, pH 5.5, or phosphate-buffered saline (PBS), pH 7.4 via dialysis. Formulations were isotonic with Osmolality measured by freezing point method between 300 and 340 mOsm/kg. Analysis by size exclusion chromatography (SEC) indicated that all proteins were ≥ 95% monomeric in these formulations. Endotoxin levels were assessed to be less than 0.1 EU per eye at the final dosing concentration.

For vitreal half-life studies, an additional pharmacokinetic study was carried out where female rabbits were administered control article (PBS, n=1), 0.15 mg/ eye AlexaFluor- 488 labeled RabFab (n=2) or AlexaFluor- 488 (AF-488) labeled RabFab-2xTSG6 at doses of 0.05 (n=2), 0.15 (n=2), and 2.5 (n=4) mg/eye in a total volume of 50 µL by ITV injection in both eyes. Test article concentration in vitreous and aqueous humor was measured at specified time points using fluorophotometry as described previously. Dickmann, L.J. et al., Invest. Ophthalmol. Vis. Sci., 56(11): 6991-6999 (2015). Concentration-time profiles were used to estimate pharmacokinetic parameters using noncompartmental analysis using Phoenix WinNonlin (Certara Inc., Mountain View, CA). For concentration-time profiles generated using fluorophotometric approaches, sampling in the first 48 hours post-dose was excluded from PK analyses due to high variability, likely attributable to interindividual variation in the site of administration and subsequent diffusion of test article through the vitreous. Dickmann, L.J. et al., Invest. Ophthalmol. Vis. Sci., 56(11): 6991-6999 (2015). PK analyses were performed using noncompartmental analysis with Clearance (CL) calculated as CL = dose/AUC, where dose is known and AUC is measured using the linear trapezoidal method. The volume of distribution at steady state was calculated as V = CL/kel, using the clearance value and the elimination rate constant obtained from the slope of the terminal phase. Elimination half-life was calculated as t½ = ln(2)/kel.

### B. Results

The capacity of HA-binding to impact ocular residence time was initially examined using pharmacokinetic (PK) experiments in New Zealand White rabbits. Although the HA concentration of rabbit vitreous (~65 µg/mL) is considerably lower than human vitreous (100-400 µg/mL), or other pre-clinical species such as pig (vitreous HA ~180 µg/mL) or cynomolgus monkey (vitreous HA ~150 µg/mL), rabbit is often employed for early PK studies following IVT dosing of test articles. Studies were designed to employ IVT injection of 0.3 mg/eye of RabFab, 0.3 mg/eye of RabFab-1xTSG6, or 0.5 mg/eye of RabFab-2xTSG6. Recovery experiments using proteins added to vitreous fluid ex vivo indicated that RabFab and RabFab-1xTSG6 could be quantitated using ELISA with anti-idotype detection antibodies as previously described (Shatz et al., 2016 Molecular Pharmaceutics). However, poor recovery was obtained with RabFab-2xTSG6 by ELISA such that radiochemical determination of vitreous concentrations was employed for this material. For PK studies, RabFab-2xTSG6 was radiolabeled with ¹²⁵iodine.

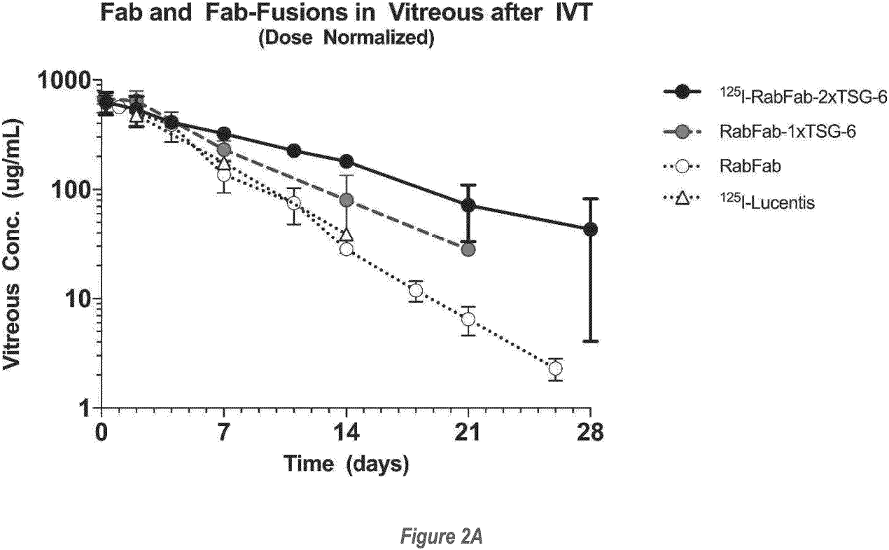

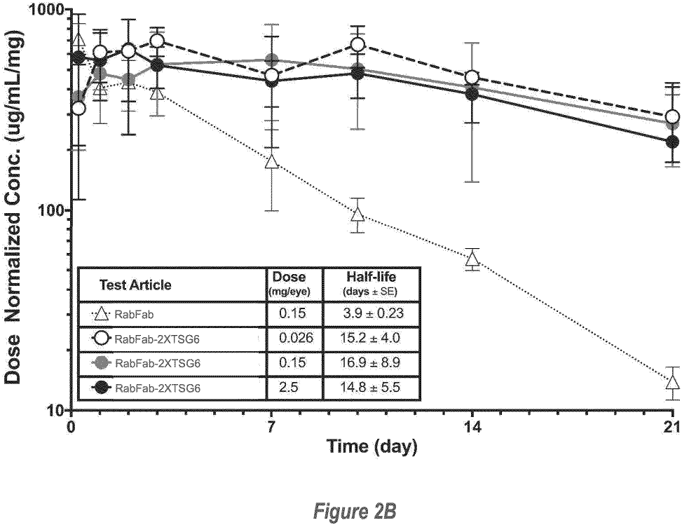

As shown in FIG. 2A, with PK parameters summarized in Table 6, both RabFab-1xTSG6 and RabFab-2xTSG6 showed longer vitreous residence time compared to the free RabFab. RabFab-1xTSG6 displayed a 1.4-fold longer half-life than RabFab whereas the increase in half-life was 2.2-fold for RabFab-2xTSG6. These results show that fusion of an Fab to an HABD can increase the retention time of these molecules in the ocular compartment. Given the higher vitreous HA concentration in other species, it is expected that even greater half-life extension would be obtained in those animals with Fab-HABDs.

TABLE 6

Rabbit PK study parameters

| SEQ ID NOS | Test article | t_{½} (days) | CL (mL/day) |
|---|---|---|---|
| 15, 16 | RabFab-2xTSG6 | 7.1 | 0.15 |
| 13, 14 | RabFab-1xTSG6 | 4.3 | 0.19 |
| 61, 62 | RabFab | 3.1 | 0.28 |

Further, vitreal half-life studies showed there was a ~3 to 4-fold increase in the vitreal half-life of RabFab-2xTSG6 compared to RabFab as observed by fluorophotometry, with no apparent dependence on dose over the range evaluated (FIG. 2B); however, the 21-day study duration was not long enough for reliable determination of pharmacokinetic parameters, with approximately 40% of the administered RabFab-2xTSG6 estimated to be remaining in vitreous at the end of the study.

### Example 5. Rabbit Ocular Tolerability of RabFab-1xTSG6 and Free TSG6

### A. Materials and Methods

The toxicity of a single ITV dose of free TSG6 and RabFab-1xTSG6 were assessed in New Zealand White rabbits. A 4-week, single IVT dose, study was designed (Table 7) and executed. Anti-drug antibodies (ADA) against RabFab-1xTSG6 or free TSG6 in serum were measured by ELISA. Plates were coated with RabFab-1xTSG-6 or free TSG6, incubated with serum collected from study animals, and then anti-drug antibodies were detected with an HRP-conjugated goat anti-rabbit Fc antibody.

TABLE 7

Rabbit ocular tolerability study (18-2244) design

| | | | | | |
|---|---|---|---|---|---|
| Group | Test article | # Animals | Dose (mg/eye) | Dose volume (µL) | Assessment |
| 1 | Free TSG6 (SEQ ID NO: 4) | 4 (2 each for necropsy at 4 and 30 days) | 0.5 | 50 | OE-Day 3, 8, 15, 22, 29 TK-Pre-dose, day 1 (1 hr, 6 hr), 2, 4, 8, 15, 22, 30 |
| 2 | RabFab-1xTSG6 (SEQ | 4 (2 each for necropsy at 4 and 30 days) | 2.0 | 50 | |
| | ID NOS: 13, 14) | | | | ADA-pre-dose, Day 4, 8, 15, 22, 30 Histopathology and electron microscopy |

OE= ophthalmic exam; TK=toxicokinetic; ADA=anti-drug antibodies

### B. Results

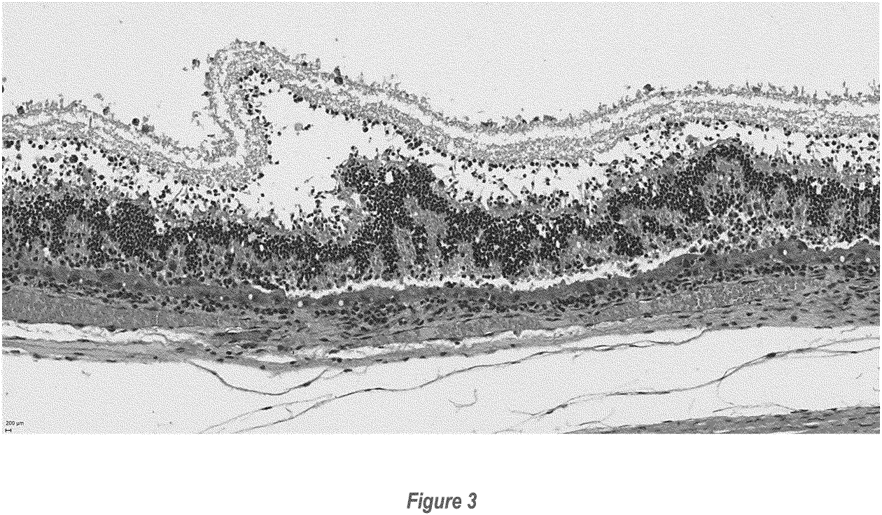

In general, animals that received RabFab-1xTSG-6 had less severe findings than those that were administered free TSG6. Animals administered free TSG6 had significant clinical observations. Although 4 animals had necropsy as scheduled on day 4, the other 4 animals were terminated early, either at day 12 or day 17 rather than day 30, due to significant clinical observations and concerns for animal welfare. These clinical observations included eyelids and conjunctiva that were swollen and red, animals kept eyes closed when approached by staff, and ocular inflammation and irritation. By 3-days post-dose, animals administered free TSG6 exhibited marked posterior incipient cataracts and variable retinal vascular attenuation, correlated with microscopic findings of lens and outer to complete retinal degeneration. Similar but less severe findings were present in animals dosed with the RabFab-1xTSG6. Marked, predominantly mononuclear cell, inflammation was noted in all animals from 7-days post-dose. Inflammation and retinal degeneration were multifocally associated with evidence of retinal detachment, and hypertrophy and peripheral migration of vimentin, glial fibrillary acidic protein (GFAP), and glutamine synthetase positive Müller cells. Histopathology image showing retinal degeneration at 4 days following IVT dosing of TSG6 is shown in FIG. 3.

Both animals administered RabFab-1xTSG6 and subjected to necropsy on day 4, had evidence of anti-drug antibodies (ADA) present in serum at day 4. However, one of these animals had ADA pre-dose whereas the remaining 3 animals in this treatment group did not have ADA pre-dose. The animals in this group that underwent later necropsy were negative for serum ADA at days 4 and 8 but became ADA positive at day 15. Analysis of serum ADA response for animals treated with free TSG6 was inconclusive due to poor sensitivity of the assay.

In general, animals that received RabFab-1xTSG6 had less severe findings than those that were administered free TSG6 (Table 8). Cataracts were present in each animal but the cataracts were punctate in nature and a correlate was not identified in microscopic sections. There was no clinical evidence of retinal degeneration, but microscopic evidence of minimal to mild outer retinal degeneration was present in individual eyes. Similar moderate to severe vitreous and aqueous cells were present from day 8 onwards. The animals were euthanized on day 4 and day 17.

TABLE 8

Microscopic lesions in animals administered 2 mg/eye RabFab-TSG6 or 0.5 mg/eye Free TSG6 bilaterally

| Animal | Day 4 | | | | End of Study | | | |
|---|---|---|---|---|---|---|---|---|
| | Free TSG6 | | RabFab-1xTSG6 | | Free TSG6 | | RabFab-1xTSG6 | |
| | 1 | 2 | 5 | 6 | 3 | 4 | 7 | 8 |
| Retinal Degeneration (OD/OS) | 2/4 | 2/4 | 0/1 | 2/0 | ⅔ | 4/2 | 2/2 | 1/1 |
| Lens Degeneration (OD/OS) | 0/2 | 2/2 | 0/0 | 0/0 | 0/0 | 3/0 | 0/0 | 0/0 |
| Inflammation (OD/OS) | 2/1 | 1/1 | 0/1 | 1/1 | 1/1 | 3/2 | 3/3 | 1/1 |

Each lesion was graded on a 5 -point scale (1-minimal to 5-severe).

Assessment of anti-RabFab responses was complicated by the presence of values above the cut off for ⅜ animals prior to dosing (2 animals administered free TSG6 and 1 animal administered RabFab-1xTSG6). However, following administration of the test items, ¾ animals administered RabFab-1xTSG6 had emergent or increasing ADA titers: 1 animal euthanized on day 4 and both animals euthanized on day 17. In contrast, only Animal 1 (necropsy day 4) administered free TSG6 had an elevated ADA titer compared to pre-dose.

The early onset of clinical signs and microscopic lesions suggest a direct role for TSG6 in retinal and lens degeneration; however, the findings at later time points were confounded by an unexpectedly vigorous ADA response. Peripheral migration of Müller cells was concluded to be a non-specific response of the rabbit retina following insult to or detachment of the retina.

### Example 6. Pharmacokinetics (PK) of Therapeutic Doses in Minipig

The objective of this study was to determine the ocular and systemic PK parameters of Fab-HABDs and Fab-HABD-HAs and the resulting extension of ocular half-life (t_{½}), administered once by IVT injection (IVT) to minipigs. In addition, investigations of anti-drug antibodies (ADAs), ocular tolerability, and ocular pathology (in some study subjects) were performed.

### A. Materials and Methods

Fourteen Göttingen SPF minipigs received therapeutic doses of the following test items into both eyes (50 µL/eye) (Table 9).

TABLE 9

Minipig pharmacokinetics (PK) study

| Test Item | VPDF (unmodified) | VPDF-2xCD44 (SEQ ID NOS: 7, 8) | VPDF-2xCD44 (SEQ ID NOS: 7, 8) + 10 kDa HA |
|---|---|---|---|
| No. of animals | 4 | 5 | 5 |
| Dose Protein [µg/eye] | 500 | 871.5 | 871.5 |
| Dose [nmol/eye] | 10.5 | 10.5 | 10.5 |
| Dose volume [µl/eye] | 50 | 50 | 50 |
| Protein concentration [mg/mL] | 10 | 17.4 | 17.4 |
| Eyes total | 8 | 10 | 10 |

After IVT dosing, blood and aqueous humor samples of test item dosed animals were collected periodically through the duration of the study (up to 9 weeks) and vitreous humor were harvested shortly after scheduled euthanasia to follow the systemic and ocular PK of the test items. Plasma, aqueous humor, and vitreous humor were analyzed for test item concentration, plasma and vitreous samples were further analyzed for the presence of ADA.

B. Results During the in-life phase of the study, macroscopic findings concerning the eyes of mainly 2 out of 5 animals that received VPDF-2xCD44 showed that IVT injections of this test item was not tolerated by the pig eyes leading to the premature sacrifice of the animals. One eye per animal was provided for histopathologic evaluation. Briefly, such macroscopic findings were: turbid vitreous, less than normal viscosity of vitreous, and finally behavioral signs of vision loss. Histopathologic findings in the eye consisted of moderate mixed cell inflammation with perivascular/vascular, predominantly mononuclear cell infiltration in the iris, ciliary body, trabecular meshwork and retina. Retinal degeneration consisted of degenerated ganglion cells, loss of cells in the INL, clumped photoreceptors and displaced nuclei in the PR layer. Furthermore, eosinophilic proteinaceous material with mixed cell infiltration and fibrous strands was observed in the vitreous. There were no findings in the optic nerve.

Macroscopic findings concerning the eyes of at least 1 out of 5 animals that received VPDF-2xCD44 + 10 kDa HA were significantly less severe in comparison to the VPDF-2xCD44. Briefly notice of a flare/white veil in the anterior chamber in both eyes which made a depot behind the cornea, but was not considered for premature termination.

In conclusion, complexation of VPDF-2xCD44 with HA (i.e., occupation of the CD44 HA-binding site with HA before IVT injection) did improve ocular tolerability of VPDF-2xCD44.

No macroscopic findings or tolerability issues were found in the group of animals that received the unmodified VPDF.

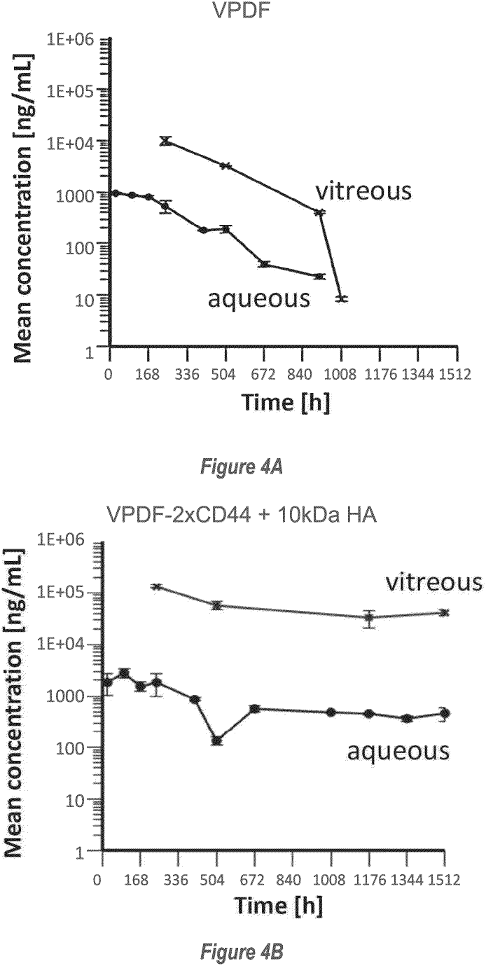

The PK results for the test items VPDF-2xCD44 and VPDF-2xCD44 + 10 kDa HA were derived from the aqueous humor and vitreous and calculated from the individual concentration time data by non-compartment analysis and are graphically presented in FIGS. 4A-B.

While the IVT t_{½} of unmodified VPDF of 5.8 days lies in the range that is expected for such a molecule, the IVT t_{½} of VPDF-2xCD44 + 10 kDa HA of 48 days corresponds to an ~8-fold increase of intraocular residence time in comparison to unmodified VPDF. In conclusion, VPDF-2xCD44 + 10 kDa shows significantly improved tolerability in comparison to VPDF-2xCD44 that is not complexed to HA and significantly improved intraocular half-life in comparison to unmodified VPDF.

### Example 7. Pre-Complexation With HA for Vitreous Compatibility

Macroscopic findings from the in vivo minipig study, i.e., turbidity of vitreous suggest an incompatibility of VPDF-2xCD44 with pig vitreous (i.e., formation of a precipitate) that can be diminished by pre-complexation of VPDF-2xCD44 with pure HA. To further investigate these effects and test if those observations are restricted to the VPDF-2xCD44 molecule or can be detected also for other Fab-HABDs, we developed ex vivo test systems to detect vitreous denaturation.

An in vitro “droplet” test was developed to assess vitreous compatibility of several Fab-HABDs when pre-complexed with HA. This Example illustrates that the Fab-HABDs that were pre-complexed with HA (i.e., the conjugates) were compatible with vitreous in in vitro experiments. Vitreous incompatibility that was observed previously may have been caused by free HABD, which was mitigated by HA pre-complexation. Incompatibility of Fab-HABDs with free HABDs was shown to be concentration dependent. Additionally, CD44ko, which is an Fab-HABD mutant comprising a point mutation that disables HA binding, was compatible with vitreous in both the pre-complexed and isolated form.

### Example 7.1. Pre-Complexation of VPDF-2xCD44 With 10 kDa HA Improves Intravitreal (IVT) Tolerability

### A. Materials and Methods

In a first test, pig vitreous was homogenized 10x in a Dounce homogenizer and cleared from debris by centrifugation at 10,000 g for 2 minutes. A 2-µl droplet of homogenized vitreous was then applied onto a glass microscopic slide. In addition, 2 µl of test sample (i.e., Fab-HABD or Fab-HABD-HA in a defined concentration) was added on top of the vitreous drop without further mixing. Approximately 1 min after merging of the drops, the sample was inspected by light microscopy at 40-fold magnification in bright-field mode for inhomogeneities and precipitation.

### B. Results

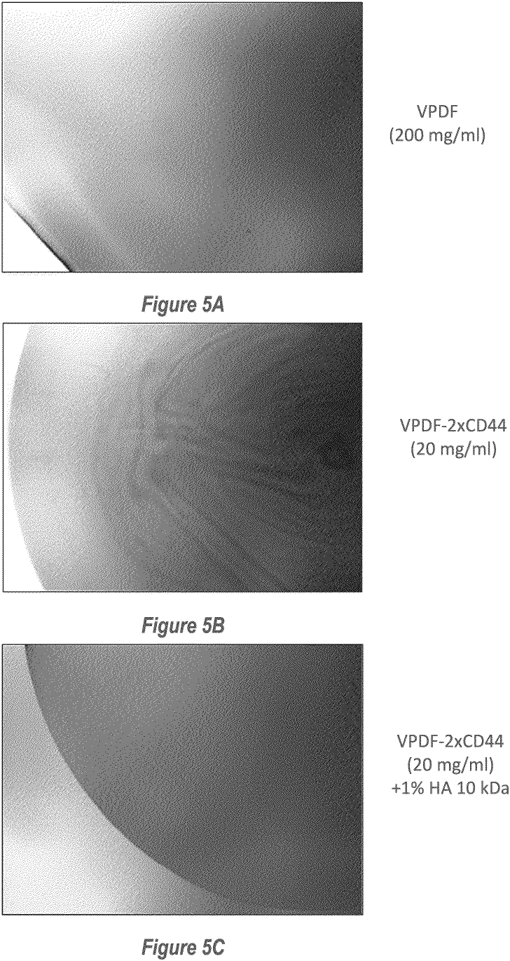

Pig vitreous that is mixed with unmodified VPDF at a concentration of 200 mg/mL in 20 mM Histidine, 140 mM NaCl, pH 6.0 is homogeneous and clear (FIG. 5A), whereas pig vitreous mixed with VPDF-2xCD44 at a concentration of 20 mg/mL in 20 mM Histidine, 140 mM NaCl, pH 6.0 is inhomogeneous and shows clear signs of precipitation (FIG. 5B).

This result suggests incompatibility of VPDF-2xCD44 with pig vitreous also upon IVT injection in vivo. Thus, vitreous incompatibility is potentially one root cause of in vivo tolerability issues seen for VPDF-2xCD44.

Pre-complexation of VPDF-2xCD44 at a concentration of 20 mg/mL with 1% (w/v) HA (10 kDa, Lifecore, Biomedical) in 20 mM Histidine, 140 mM NaCl, pH 6.0 leads to vitreous compatibility (FIG. 5C). This result reflects findings from the minipig in vivo study described above, where it was shown that pre-complexation of VPDF-2xCD44 with 10 kDa HA improves IVT tolerability.

### Example 7.2. Vitreous Incompatibility of VPDF-2xCD44 Is Dependent on Concentration

### A. Materials and Methods

To test concentration dependency of vitreous incompatibility of VPDF-2xCD44, 2 µl of pig vitreous was mixed with 1:4 dilutions of VPDF-2xCD44 in 20 mM histidine, 140 mM NaCl, pH 6.0 at a starting concentration of 37.5 mg/mL. Mixtures of vitreous and protein were examined by light microscopy for vitreous inhomogeneities.

### B. Results

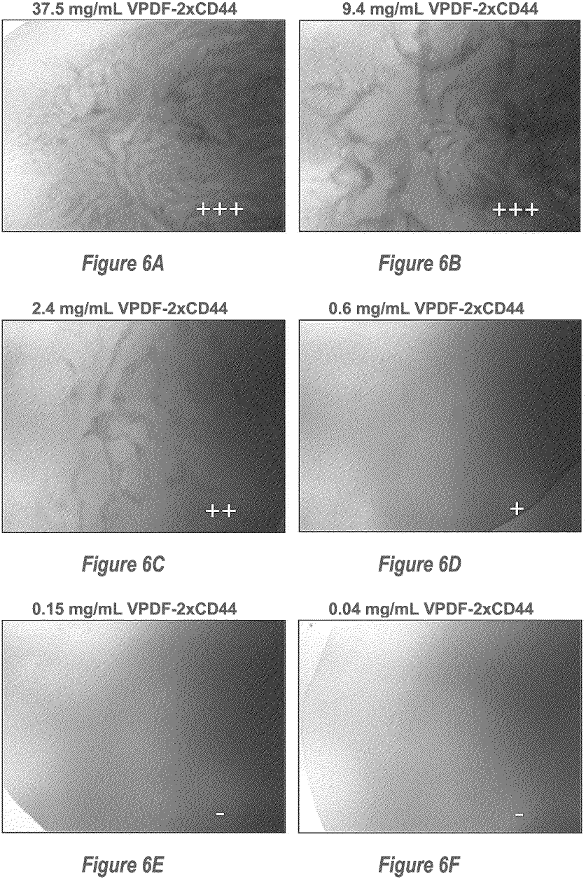

Detected inhomogeneities were dependent on the protein concentration (Table 10; FIGS. 6A-F). Vitreous compatibility of VPDF-2xCD44 was reached between 0.6 to 0.15 mg/mL. Relating these results to findings in the in vivo minipig study described above (concentration of VPDF-2xCD44 = 17.4 mg/mL) suggests that IVT injection of a VPDF-2xCD44 solution at a concentration of 17.4 mg/mL might lead to similar inhomogeneities that might be a root cause for observed tolerability issues.

TABLE 10

Concentration dependency of vitreous incompatibility of VPDF-2xCD44 (SEQ ID NOS: 7, 8)

| | | | | | | |
|---|---|---|---|---|---|---|
| VPDF-2xCD44 concentration [mg/mL] | 37.5 | 9.4 | 2.4 | 0.6 | 0.15 | 0.04 |
| Vitreous inhomogeneity | +++ | +++ | ++ | + | - | - |

+++ strong / ++ medium / + light / - clear

### Example 7.3. Vitreous Incompatibility of VPDF-2xCD44 Relates to its Interaction With Intravitreal (IVT) HA

### A. Materials and Methods

To test if vitreous incompatibility of VPDF-2xCD44 is induced by interaction of VPDF-2xCD44 with IVT HA we designed a variant of this molecule (VPDF-2xCD44-ko) that contains a point mutation within the HA-binding site of CD44 that abolishes binding to HA while leaving the rest of the protein intact (herein termed “ko variant”).

### B. Results

The CD44ko variant showed identical behavior in transient expression, purification and biophysical characteristics (analytical size exclusion, denaturing SDS capillary electrophoresis) and its identity was confirmed by mass spectrometry. Introduction of the HA-binding site mutation resulted in a complete loss of affinity as shown by SPR (tested with the same method as in Example 2).

When this VPDF-2xCD44-ko variant was tested for vitreous compatibility as described in Example 7.2 above at the same concentration as 2x VPDF, no vitreous inhomogeneity was detected, suggesting vitreous compatibility (Table 11).

TABLE 11

Vitreous compatibility of VPDF-2xCD44 and corresponding knock-out variant

| | | |
|---|---|---|
| Molecule | VPDF-2xCD44 (SEQ ID NOS: 7, 8) | VPDF-2xCD44ko (SEQ ID NOS: 25, 26) |
| Concentration [mg/mL] | 20 | 20 |
| Vitreous inhomogeneity | +++ | - |

++ strong / ++ medium / + light / - clear

### Example 7.4. VPDF-2xCD44 Is Compatible With Vitreous After Pre-Treatment With Hyaluronidase

### A. Materials and Methods

Additionally, we tested vitreous compatibility of VPDF-2xCD44 in pig vitreous that was pre-treated with hyaluronidase to degrade HA. For this, hyaluronidase from pig testes (Sigma) was dissolved at 2 mg/mL (> 1.5 U/µL) in PBS. 1 µL of this hyaluronidase solution was added to 50 µL pig vitreous and incubated for 2 hours at 37° C. A control sample was treated with PBS buffer only.

### B. Results

As a result, VPDF-2xCD44 did not show inhomogeneity at a concentration of 20 mg/mL when mixed with vitreous that was pre-treated with hyaluronidase, likely due to degradation of high molecular weight HA.

In summary these results suggest that vitreous incompatibility that can be a root cause for in vivo tolerability issues of VPDF-2xCD44 relates to interaction of the CD44-HABD with high molecular weight IVT HA.

### Example 7.5. Vitreous Incompatibility of Fab-HABD Relates to the Interaction of HABD With Vitreal HA at Certain Concentrations

### A. Materials and Methods

To test if vitreous incompatibility is a feature of VPDF-2xCD44 only, we tested other Fab-HABDs or HABDs alone for vitreous inhomogeneity as described above in Examples 7.2 and 7.3 above. The proteins that were tested are described in Example 1.

### B. Results

VPDF-1xCD44 showed comparable vitreous inhomogeneity compared to VPDF-2xCD44. The results suggest that increase in avidity and potential crosslinking of HA-polymers by the 2x version are not related to vitreous incompatibility. The results suggest that the interaction between the CD44 HABDs with the IVT HA that is related to vitreous incompatibility (Table 12).

TABLE 12

Vitreous compatibility of indicated molecules comprising HABD TSG6 compared to VPDF-1xCD44

| | | | | | | |
|---|---|---|---|---|---|---|
| Molecule | VPDF-1xCD44 | G6.31 | G6.31-1xTSG6 | G6.31-2xTSG6 | NVS24-1xTSG6 (Lava12) | TSG6 |
| SEQ ID NOS | 5, 6 | 103, 104 | 17, 18 | 19, 20 | 21, 22 | 4 |
| Concentration [mg/mL] | 20 | 5 | 5 | 2.3 | 5 | 5 |
| Vitreous inhomogeneity | +++ | - | ++ | ++ | ++ | ++ |

++ strong / ++ medium / + light / - clear

Fab-HABDs with TSG6 domains show comparable vitreous inhomogeneity as Fab-HABDs with CD44. The Fab component G6.31 did not show vitreous inhomogeneity at the same concentration, whereas the TSG6-domain in isolation did. This again supports the suggestion that vitreous incompatibility is related to the interaction between HABD and vitreous HA at a certain concentration.

### Example 7.6. Vitreous Incompatibility Can Be Rescued by Pre-Complexation With HA

### A. Materials and Methods

To test if vitreous incompatibility that was detected for VPDF-1xCD44 and TSG6-variants can be rescued by pre-complexation with HA, we generated the conjugates shown in Table 13 with 1 % (w/v) HA (10 kDa, Lifecore, Biomedical).

### B. Results

Vitreous inhomogeneity was rescued for all Fab-HABDs tested by pre-complexation with 10 kDa HA (Table 13). These results suggest that pre-complexation of HA-binding proteins with pure HA can be a method to improve vitreous compatibility and thus potential tolerability issues of these molecules.

TABLE 13

Vitreous compatibility of Fab-HABDs comprising TSG6 HABD pre-complexed with 1% HA 10 kDa

| | | | | | | |
|---|---|---|---|---|---|---|
| Fab-HABD + 1% HA 10 kDa | VPDF-1xCD44 | G6.31Fab | G6.31-1xTSG6 | G6.31-2xTSG6 | NVS24-1xTSG6 (Lava12) | TSG6 |
| SEQ ID NOS | 5, 6 | 103, 104 | 17, 18 | 19, 20 | 21, 22 | 4 |
| Concentration [mg/mL] | 20 | 5 | 5 | 2.3 | 5 | 5 |
| Vitreous inhomogeneity | - | - | - | - | - | - |

++ strong / ++ medium / + light / - clear

### Example 7.7. Vitreous Incompatibility of Fab-HABDs Is Not Specific to Pig Vitreous

### A. Materials and Methods

To test if vitreous incompatibility that was detected for CD44- and TSG6- containing Fab-HABDs is an effect that occurs only in pig vitreous, we carried out compatibility tests as described in Examples 7.1-7.6 above using rabbit vitreous instead of pig vitreous.

### B. Results

Identical vitreous incompatibility was detected as for pig vitreous for all tested Fab-HABDs. In addition, all vitreous incompatibility that was detected in rabbit vitreous could be rescued by pre-complexation of Fab-HABDs with 10 kDa HA.

These results suggest that vitreous incompatibility is not specific to pig vitreous.

### Example 7.8. Vitreous Inhomogeneity Is Induced Upon Injection In Iivo

### A. Materials and Methods

To generate a link between the ex vivo vitreous compatibility test results and the tolerability findings from the in vivo minipig study, we tested if vitreous inhomogeneity and rescue by HA-pre-complexation can be detected in a whole pig eye.

For this, whole pig eyes were received immediately after slaughter and injected with 50 µl of a VPDF-2xCD44 solution in 20 mM Histidine, 140 mM NaCl, pH 6.0 at a concentration of 17.4 mg/mL +/- 1% (w/v) HA 10 kDa. Eyes (identical to in vivo minipig study described above). Eyes where then transferred to HBSS (Lonza, Biowhittaker) and kept at 37° C. for 4 h. After incubation, eyes were opened, vitreous was removed investigated for inhomogeneity.

### B. Results

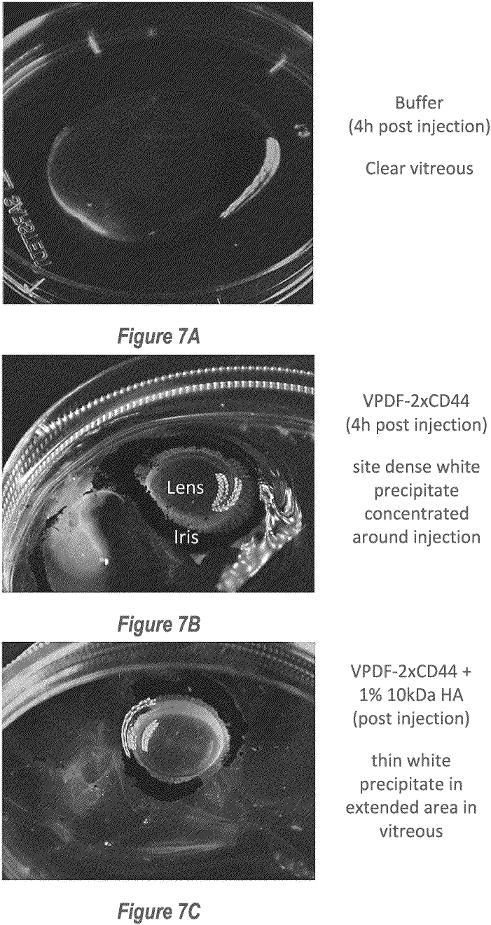

As shown in FIGS. 7A-C, vitreous incompatibility upon injection can be resolved by pre-complexation of Fab-HABDs with HA. Injection of buffer did not lead to IVT inhomogeneity and resulted in a clear vitreous (FIG. 7A). Injection of VPDF-2xCD44 resulted in dense white inhomogeneity (precipitate-like) in the vitreous around the injection side (FIG. 7B). Vitreous from eyes that were injected with VPDF that was pre-complexed with pure HA was showed significant differences (FIG. 7C): although inhomogeneity was detected, this was significantly less dense and thinner throughout the vitreous.

These results suggest that VPDF-2xCD44 induced inhomogeneity occurs also in a whole pig eye in the vicinity of the injection site. Without being bound to this theory, we suggest that the same inhomogeneity is induced upon injection in vivo and might be a root cause for the observed tolerability issues.

Additionally, pre-complexation of VPDF-2xCD44 with HA reduces the observed inhomogeneity around the injection site. We suggest that the same effect leads to the improved tolerability that was observed in vivo for VPDF-2xCD44-HA. Together with the observations in Examples 7.5 and 7.6 that vitreous incompatibility occurs also with another TSG6 HABD and equally can be rescued by pre-formulation with pure HA, we suggest that this approach can be a general principle to improve vitreous compatibility and thus IVT tolerability of HABD containing proteins.

### Example 8. Versican VG1 and VG1ΔIg HABDs Are Capable of Binding HA

HABDs of Versican were studied to determine if they could be used as HABDs that provide ocular tolerability and ocular residence time that are superior to those of TSG6 and CD44 HABDs.

Versican was identified as having a tandem repeat of link modules. As shown in FIG. 8A, the amino acid sequence of Versican encodes an Ig-like domain followed by two link modules such that an N-terminal fragment of Versican, herein named WT VG1, comprises an N-terminal Ig-like domain and 2 link domains. In this Example, we produced WT VG1 and a truncated variant without the Ig domain, VG1ΔIg, and tested them for binding to HA. Additionally, in the following Examples, WT VG1 and Fab-HABDs consisting of a Fab and WT VG1 were tested for in vitro vitreous compatibility, and tolerability upon IVT injection in rabbits and mini-pigs.

### A. Materials and Methods

Expression plasmids for the proteins were generated by restriction cloning and/or gene synthesis using standard molecular biology techniques. Expression was performed in either CHO or HEK293 cells.

Supernatants were harvested by centrifugation at 4,000 rpm, 4° C., for 20 minutes. Thereafter, cell-free supernatant was filtered through a 0.22 µm bottle-top-filter and stored in a freezer (-20° C.).

His-tagged mutants of WT VG1 and VG1ΔIg were purified from cell culture supernatants by affinity chromatography using Ni-NTA resin together with SEC. Briefly, sterile-filtered cell culture supernatants were captured on HisTrap resin, washed and eluted using buffer containing high imidazole concentration. The eluted protein fractions were pooled and concentrated before subjected to SEC using 20 mM Histidine Acetate, 150 mM NaCl, pH 5.5 as running buffer.

Binding of WT VG1 and VG1ΔIg to HA was tested by SPR using a Biacore T200 instrument (GE Healthcare). Briefly, the WT VG1 and VG1ΔIg were injected for 80 sec or 120 sec onto a Series S CM5 chip (GE Healthcare Life Science Solutions) indirectly coated with biotin-HA (Creative PEGWorks, North Carolina) through immobilized streptavidin. Injection concentrations ranged from 0.5 nM to 1 µM each. The dissociation phase was monitored for 300 sec to 600 sec. Subsequently, the surface was regenerated by injecting 1 M MgCl₂ for 15 seconds. All experiments were performed at 25° C. using PBS (10 mM Na₂HPO₄, 1 mM KH₂PO₄, 137 mM NaCl, 2.7 mM KCl pH 7.4). The derived curves for HA binding were fitted to a 1:1 Langmuir binding model using the BIAevaluation software.

### B. Results

Versican HABDs are capable of binding HA. The K_{D} for HA binding for each protein is shown in Table 14.

TABLE 14

HA binding by SPR for WT VG1 and VG1ΔIg fragment

| SEQ ID NO | Protein | K_{D} [µM] |
|---|---|---|
| 29 | WT VG1 | 0.17 |
| 32 | VG1ΔIg | 0.23 |

### Example 9. Glycosaminoglycan Binding Profile of WT VG1 and TSG6 Proteins

### A. Materials and Methods

The glycosaminoglycan (GAG) binding profile of WT VG1 and TSG6 was determined by measuring binding to heparin sulfate and chondroitin sulfate by SPR using a Biacore T200 instrument (GE Healthcare). Briefly, the proteins were injected for 180 sec onto a Series S CM5 chip (GE Healthcare Life Science Solutions) coated indirectly with either biotin-heparin sulfate or biotin-chondroitin sulfate through streptavidin. Injection concentrations ranged from ~ 5 nM to 1000 nM each. The dissociation phase was monitored for 120 sec. Subsequently, the surface was regenerated by injecting 1 M MgCl₂ for 30 seconds.

### B. Results

The results indicate that the WT VG1 is more selective in binding than TSG6 (Table 15). WT VG1 had no observable binding to heparin sulfate or chondroitin sulfate while TSG6 had tight binding for both heparin and chondroitin sulfate.

TABLE 15

Heparin sulfate and chondroitin sulfate binding by SPR

| SEQ ID NO | Molecule | Heparin sulfate binding K_{D} [µM] | Chondroitin sulfate binding K_{D} [µM] |
|---|---|---|---|
| 29 | WT VG1 | No binding | No binding |
| 4 | TSG6 | 0.01 | 0.01 |

### Example 10. Fab-HABDs Comprising VG1 HABDs Are Capable of Binding Antigen and HA

### A. Materials and Methods

### A.1. Construct Design

Fab-HABDs were or may be generated through recombinant fusion of the WT VG1 sequence to the C-terminus of the Fab fragment heavy chain or N-terminus of the IgG1 heavy chain. For the peptide-VG1 fusions, constructs with the peptide (EETI) attached at both N-terminus of WT VG1 (EETI-VG1) and C-terminus of WT VG1 (VG1-EETI) were or may be generated. Two additional constructs with TEV cleave sites incorporated between EETI and WT VG1 (EETI-TEV-VG1 and VG1-TEV-EETI) were also generated. The linkers used or that may be used are shown in Table 16.

TABLE 16

Linker sequences

| Linker SEQ ID NO | Protein | Linker |
|---|---|---|
| 27 | RabFab-VG1 | GGGGS |
| 27 | PigFab-VG1 | GGGGS |
| 27 | G6.31.Fab-VG1 | GGGGS |
| 27 | VPDF-VG1 | GGGGS |
| 82 | VG1-Fc (2x)* | RKCLIPFGNSVT |
| 27 | VPDF-VG1ΔIg (prophetic construct) | GGGGS |
| 88 | 20D12v2.3-VG1 | GGGGSGGGGS |
| 88 | Ranibizumab-VG1 | GGGGSGGGGS |
| 83 | Anti-HtrA1-VG1 | GGGG |
| 84 | EETI-VG1 | GSGSGSGSGS |
| 85 | EETI-TEV-VG1 | ENLYFQGSGSGSGSGS |
| 84 | VG1-EETI | GSGSGSGSGS |
| 85 | VG1-TEV-EETI | ENLYFQGSGSGSGSGS |

* Fusing VG1 with Fc makes the fusion protein a homodimer, which results in 2 copies of VG1 per molecule. Hence, the notation “2x” in the protein name.

### A.2. Generation of Species-Matched Surrogate Pig Anti-VEGF Fab

As searching of the abYsis database yielded no known instances of paired heavy and light chains for a pig (Sus scrofa) IgG, we sought to generate an actively binding antibody to a known antigen by CDR grafting from an anti-VEGF Fab (G6.31.AARR). The NCBI Expressed Sequence Tag (EST) database was searched for porcine mRNA ESTs with high sequence identity to the G6.31 framework (V_{H}4/V_{L}K2). Several sequences were selected and the CDRs from G6.31.AARR were grafted within the appropriate framework regions to generate “porcinized” G6.31.AARR. Heavy and light chain sequences were randomly paired and expressed in 293Expi or CHO cells in 30 mL culture. Purification was performed on Capto L resin followed by size-exclusion chromatography and purified proteins were examined by SDS-PAGE, mass-spec, and evaluated for binding to human and pig VEGF. One sequence with good affinity for VEGF was selected for scale up and subsequent tox/PK analysis and this sequence was also recombinantly fused to VG1 to generate PigFab-VG1.

### A.3. Protein Expression and Purification

Protein expression was performed by cationic lipid transfection of DNA constructs into CHO or 293Expi cells. Culture volumes ranged from 30 mL to 35 L. For some constructs, fast stable cell lines were generated to increase protein yield per culture volume.

Purification was performed by affinity chromatography using either Ni-NTA resin for 6x-Histidine-tagged molecules, or Gamma bind Plus resin for Fab fusions. In some cases, a secondary ion exchange step was performed prior to a final size-exclusion step on Sephadex resin.

### A.4. HA Binding

In order to confirm that VG1 retained its HA binding properties as a Fab-HABD, SPR was used as previously described in Example 2.1. Experiments were conducted using single cycle kinetics and dissociation monitored for up to 600 sec. The protein concentrations tested varied between proteins but ranged between 500 nM and 6.25 nM.

### A.5. Antigen Binding

Antigen binding was tested by directly immobilizing the respective antigen onto a Series S CM5 chip (GE Healthcare) and measuring binding by SPR as described in Example 2.1. Different protein concentrations were used based on the known affinity of the interaction.

### B. Results

### B.1. Hyaluronan (HA) Binding

All data for HA binding was fit to the 1:1 Langmuir binding model using the BIAevaluation software. The K_{D} for each protein is shown in Table 17.

TABLE 17

HA-binding of VG1 Fab-HABDs

| SEQ ID NO | Protein | K_{D} [µM] |
|---|---|---|
| 63, 64 | RabFab-VG1 | 0.13 |
| 65, 66 | PigFab-VG1 | 0.12 |
| 67, 68 | G6.31.Fab-VG1 | 0.15 |
| 69, 70 | VPDF-VG1 | 0.12 |
| 118, 119 | Anti-HtrA1-VG1 | 0.08 |
| 71 | VG1-Fc (2x) | 0.017 |
| 78 | EETI-VG1 | 0.16 |
| 79 | EETI-TEV-VG1 | 0.22 |
| 80 | VG1-EETI | 0.16 |
| 81 | VG1-TEV-EETI | 0.18 |
| 93 | VC072M.GS10X.V G1CTH | 0.0082 |
| 94 | VG1NTH.GS10X. VC072M | 0.033 |

### B.2. Antigen Binding

Table 18 shows the proteins that were analyzed for antigen binding along with measured K_{D}. C-terminal fusion of VG1 with various Fab heavy chains did not impact antigen binding. For the EETI-VG1 fusion, the flexibility of the linker and site of attachment impacted antigen binding. A more flexible linker and C-terminus fusion was preferred.

TABLE 18

Antigen binding

| SEQ ID NO | Protein | Antigen | K_{D} [µM] |
|---|---|---|---|
| 63, 64 | RabFab-VG1 | Not applicable | Not applicable |
| 65, 66 | PigFab-VG1 | VEGF | Not determined |
| 67, 68 | G6.31.Fab-VG1 | VEGF | 0.0001 |
| 69, 70 | VPDF-VG1 | VEGF | Not determined |
| 71 | VG1-Fc (2x) | Not applicable | Not applicable |
| 78 | EETI-VG1 | Trypsin | 0.43 |
| 79 | EETI-TEV-VG1 | Trypsin | 0.14 |
| 80 | VG1-EETI | Trypsin | 0.013 |
| 81 | VG1-TEV-EETI | Trypsin | 0.007 |
| 93 | VC072M.GS10X.V G1CTH | VEGF | 0.00075 |
| 94 | VG1NTH.GS10X. VC072M | VEGF | 0.00055 |

### Example 11. In Vitro Vitreous Compatibility of HABDs

### A. Materials and Methods

This Example describes the testing of VG1 domain solubility in vitreous fluid. Vitreous fluid, prepared using a Dounce homogenizer followed by centrifugation at 10,000 × g for 2 minutes to remove debris, were used for these studies.

Additional experiments utilized Alexa488-labeled proteins such that both bright field and fluorescence microscopy could be used to monitor precipitation in vitreous fluid ex vivo. Equal volumes of test article and vitreous fluid were mixed by successive injection into each of two channels of a 3-in-1 tri-channel □-slide (ibidi, USA, Inc. Cat#80316) and the mixing interface was monitored visually by microscopy.

### B. Results

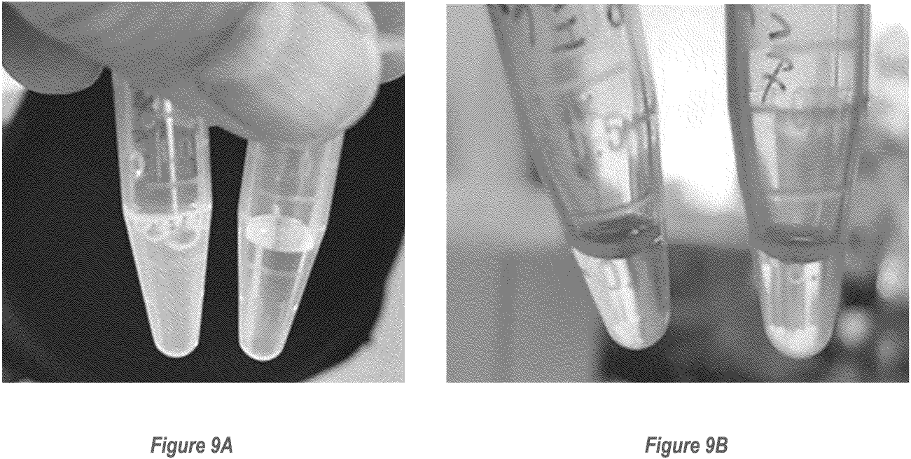

Upon mixing of TSG6 with pig vitreous fluid, previously diluted 1:4 with PBS pH 7.4, the solution became turbid (FIG. 9A) and a pellet was observed upon centrifugation of the mixture. In contrast, the solution remained clear upon mixing VG1 with pig vitreous in both 1:4 and 1:1 ratios (FIG. 9B) and no pellet was observed upon centrifugation.

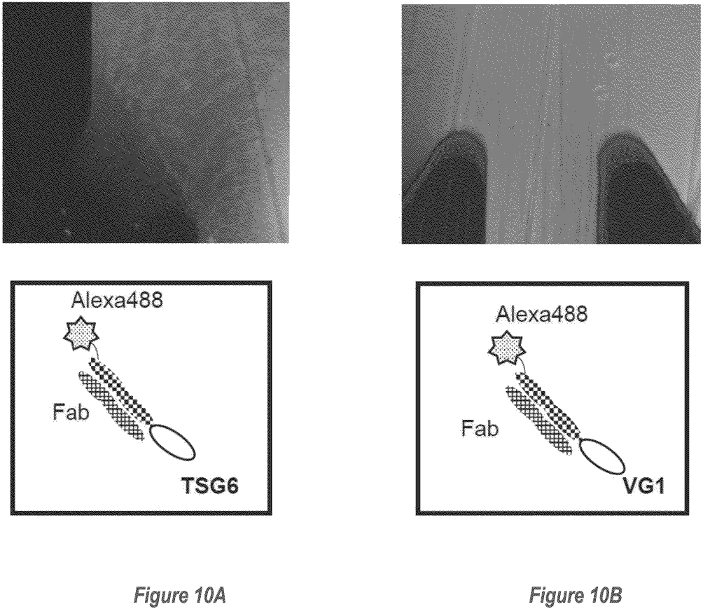

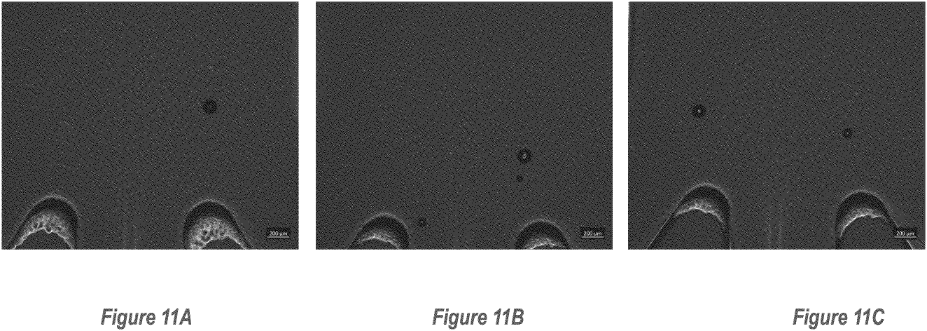

Further, precipitation was observed for RabFab-TSG6 in pig vitreous ex vivo (FIG. 10A) whereas no precipitation was observed for RabFab-VG1 (FIG. 10B). Similarly, no precipitation was observed in rabbit vitreous ex vivo when comparing VG1 (FIG. 11A), RabFab-VG1 (FIG. 11B), or a formulation containing equal concentrations (mass basis) of RabFab-VG1 and 10 kDa HA (FIG. 11C).

In contrast to the TSG6, pre-formulation of VG1 with 10 kDa HA is not always required to prevent precipitation in vitreous fluid ex vivo.

### Example 12. Interaction of VG1 With Vitreous Fluid Ex Vivo

### A. Materials and Methods

Fluorescence correlation spectroscopy (FCS) was used to examine the interaction of isolated VG1 and Fab-VG1 Fab-HABDs with vitreous humor ex vivo. VG1 and Fab-VG1 were covalently labeled on lysine residues using PEG4-DY647-N-hydroxysuccinimide ester. The fluorescence emission of DY647 can be excited by lasers of 594 or 633 nm and detected at longer wavelengths. The reaction chemistry was controlled such that labeling level was not greater than 1 fluorescent dye per molecule. Porcine vitreous humor was collected from eyes of freshly slaughtered animals and homogenized using a dounce homogenizer. This material was serially diluted 1:3 with phosphate-buffered saline (PBS) pH 7.4. A labeled test article was added to each diluted aliquot to a final concentration of 20 nM. Test articles were (1) free VG1, (2) pigFab-VG1, (3) pigFab-VG1 mixed with 1:1 equal weight ratio 10 kDa HA, (4) RabFab-VG1, and (5) RabFab-VG1 mixed with 1:1 equal weight ratio 10 kDa HA. After a 2-hour incubation at ambient temperature, FCS was performed.

### B. Results

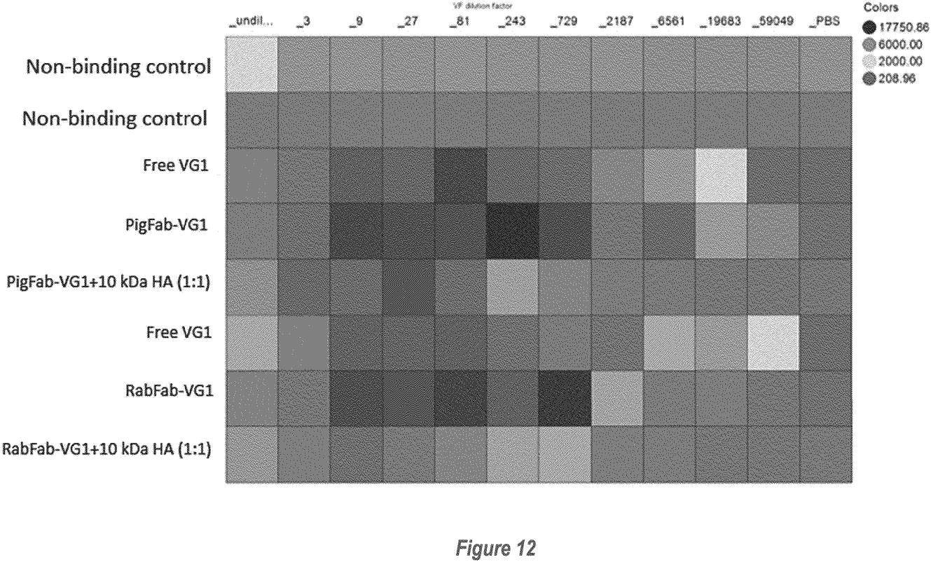

Results of FCS measurements are shown in FIG. 12. All of the samples when incubated with undiluted or slightly diluted vitreous showed significantly retarded diffusion relative to an incubation in buffer (PBS) alone. For free VG1, PigFab-VG1 and RabFab-VG1, this retarded diffusion persisted until the vitreous was diluted more than 6,000-fold (FIG. 12, rows 3, 4, 6, and 7; from undiluted to dilution factor 6,561). Slow diffusion was also observed for the samples co-formulated with 10 kDa HA but the effect disappeared when the fold dilution of vitreous fluid was ≥729-fold (FIG. 12, row 5: PigFab-VG1+10 kDa HA (1:1), and row 8: RabFab-VG1+10 kDa HA (1:1); from dilution factor 729 to PBS). These results indicate that there is a strong interaction between vitreous components, most likely high molecular weight HA endogenous to the vitreous humor, and VG1 containing test articles. Even in the presence of low molecular weight HA, and at smaller dilutions of vitreous fluid (FIG. 12, row 5: PigFab-VG1+10 kDa HA (1:1), and row 8: RabFab-VG1+10 kDa HA (1:1)), the VG1 can interact with endogenous HA. This indicates that VG1 and Fab-VG1 can dissociate from 10 kDa HA and bind to the HA present in vitreous fluid. However, once the vitreous fluid is significantly diluted there is not a high enough concentration of high MW HA to compete for VG1 binding to low MW HA (FIG. 12, row 5: PigFab-VG1+10 kDa HA (1:1), and row 8: RabFab-VG1+10 kDa HA (1:1); from dilution factor 729 to PBS). VG1 bound to low MW HA experiences a small or negligible slowing of diffusion relative to unbound material (FIG. 12, PBS control for row 5: PigFab-VG1+10 kDa HA (1:1), and row 8: RabFab-VG1+10 kDa HA (1:1); these samples have 10 kDa HA without added vitreous).

### Example 13. Effect of Pre-Complexation With 10 kDa HA on Thermal Stress Stability of Fab-VG1

### A. Materials and Methods

The effect of pre-complexation of Fab-VG1 with 10 kDa HA upon stability to thermal stress was tested using anti-HtrA1-VG1 protein. For these experiments, anti-HtrA1-VG1 was formulated at 3 mg/mL in phosphate-buffered saline (PBS) pH 7.4, and with or without addition of 10 kDa HA at 1.8 mg/mL. The 1.8 mg/mL (180 µM) concentration of 10 kDa HA is a 5-fold molar excess over the anti-HtrA1-VG1 concentration (35 µM). These formulations were incubated at a temperature of 37° C. for 4 weeks and then analyzed by non-reduced capillary electrophoresis-sodium dodecyl sulphate (NR CE-SDS) as described by Michels et al., 2007 (Anal. Chem. 79, 5963). In addition to monomeric species and fragments, aggregates resistant to denaturation by SDS are detected by NR CE-SDS.

### B. Results

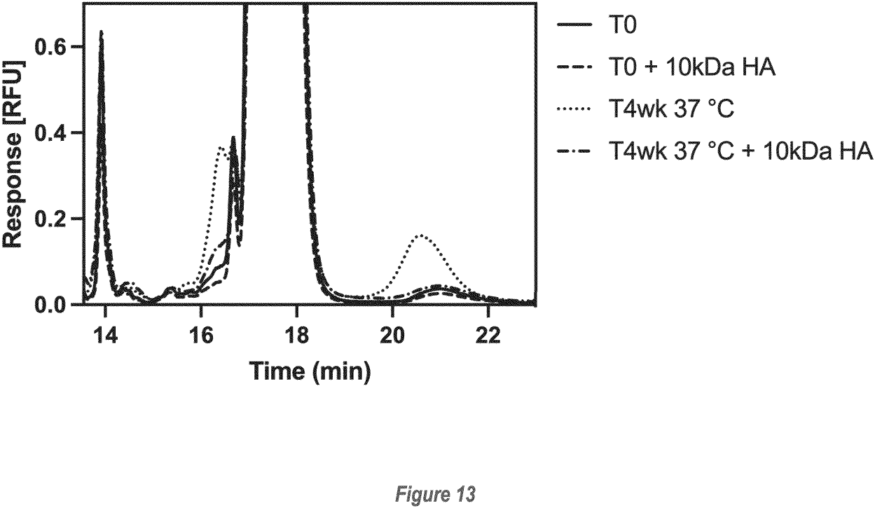

As shown in FIG. 13, and summarized in Table 19, pre-complexation with 10 kDa HA inhibits the formation of SDS-stable aggregates in anti-HtrA1-VG1. The rate of formation of high molecular weight forms (HMWF) is reduced from 1.2% per week to 0.1 % per week. The presence of 10 kDa HA also seems to have an effect on fragmentation, albeit smaller than the effect on aggregation, with the rate of formation of low molecular weight forms (LWMF) decreasing by about 2-fold when complexed to HA. These results indicate that inclusion of 10 kDa HA in the formulation stabilized anti-HtrA1-VG1 towards thermal stress conditions at neutral pH.

TABLE 19

Summary of analysis of thermal stress samples by NR CE-SDS

| Sample | % HMWF | % Main Peak | % LWMF |
|---|---|---|---|
| Anti-HtrA1-VG1 - no incubation control | 1.2 | 92.8 | 6.0 |
| Anti-HtrA1-VG1 - after 4-week incubation | 6.0 | 80.3 | 13.8 |
| Anti-HtrA1-VG1 + 10 kDa HA - no incubation control | 1.1 | 93.0 | 5.8 |
| Anti-HtrA1-VG1 + 10 kDa HA - after 4-week incubation | 1.5 | 88.8 | 9.7 |

### Example 14. Ocular Tolerability of VG1 and VG1 Fab-HABDs in Göttingen Minipig®

### A. Materials and Methods

### A.1. Intravitreal (IVT) Injections and Evaluations of End Points

The tolerability of IVT injection of VG1 and Fab-VG1 Fab-HABD was evaluated using Göttingen Minipig®. The design of the study is shown in Table 20.

TABLE 20

Design of ocular tolerability study of VG1 and Fab-HABDs in minipig

| Group No. | Test Article^{a} | Dose Level (mg/eye) | Dose Volume (µL/eye) | Dose Concentration (mg/mL) | No. of Males | |
|---|---|---|---|---|---|---|
| | | | | | Day 4 Necropsy | Day 30 Necropsy |
| 1 | Vehicle Control | 0 | 50 | 0 | 3 | - |
| 2 | WT VG1 (SEQ ID NO: 29) | 1.13 | 50 | 22.5 | 2 | 4 |
| 3 | PigFab-VG1 (SEQ ID NOs: 65, 66) | 1.8 | 50 | 36 | 2 | 4 |
| 4 | PigFab-VG1 + 10 kDa HA | 1.8 | 50 | 36 | 2 | 4 |

^{a} All test articles and vehicle control was administered via one bilateral IVT injection on Day 1.

Each minipig received a single injection of 50 µL administered via IVT in both eyes. Based on historical data, this volume was well tolerated in minipigs. The IVT injection procedure was performed by a boardcertified veterinary ophthalmologist. Group 1 minipigs were treated with injections of the vehicle control. Group 2 minipigs were treated with the isolated WT VG1 (produced as described above in Example 8). Group 3 minipigs were treated with pigFab-VG1 (produced as described in Example 10) Group 4 minipigs were treated with pigFab-VG1 pre-formulated with an equal weight of 10 kDa HA. All test articles were formulated in 20 mM Histidine Acetate, 150 mM NaCl, pH 5.5, at the indicated protein concentrations. The dose of pigFab-VG1 in groups 3 and 4 represents the maximal feasible dose that keeps the total endotoxin level at less than 0.05 endotoxin units (EU) per eye. This level of endotoxin has been found previously to be tolerated in mini-pig ocular studies. Given the difference in molecular weights between WT VG1 (~30 kDa) and pigFab-VG1 (~80 kDa), the dose level in Group 2 represents a 1.6 HA-binding molar equivalents per dose compared to groups 3 and 4.

The following parameters and end points were evaluated in this study: mortality, clinical signs, body weights, ophthalmology (examinations, intraocular pressure measurements, wide-field color fundus imaging, OCT imaging and electroretinography [ERG]), bioanalytical analysis, toxicokinetic parameters, anti-drug antibody evaluations, gross necropsy findings, and histopathologic examinations.

Ophthalmoscopic examinations were conducted on both eyes of all surviving animals by a board-certified veterinary ophthalmologist via indirect ophthalmoscopy and slit-lamp biomicroscopy. Ophthalmic exams were conducted on all animals before treatment, and on Days 1 (post dose), 3, 5, 8, 15, 17, 22 and 29.

Intraocular pressure (IOP) was measured by applanation tonometry on both eyes of all surviving animals by a board-certified ophthalmologist at the same time as ophthalmic examinations. Intraocular pressure was measured on all animals before treatment, and on Days 1 (post dose), 3, 5, 8, 15, 17, 22, and 29.

Wide-field, color fundus imaging using the Clarity RetCam Shuttle was conducted on all surviving animals on Day 29. The administered test article, if visible, was attempted to be photographed.

On Day 29, optical coherence tomography imaging using the Heidelberg Spectralis HRA/OCT system; single, vertical, high-resolution line scan through optic nerve was performed.

The ERG assessments were conducted on all surviving animals on Day 29. The animals were dark adapted for a minimum of 1 hour prior to ERGs. Full-field flash ERGs with Ganzfeld dome stimulus, with flash intensities according to ISCEV standard parameters and light adaptation time of 5 minutes (Retiport Gamma, Roland Consult); amplitude and latency values were measured from tracings.

Blood samples (approximately 0.5 mL) were collected from all surviving animals via the anterior vena cava through the thoracic inlet for determination of the serum concentrations of the test article. The animals were not fasted prior to blood collection with the exception of the intervals that coincided with fasting for other procedures. Blood collections occurred once pretreatment, Day 1 (6 and 12 hours postdose), and Days 2, 3, 5, 8, 12, 15, 22, and 29.

Blood samples were collected in serum separator tubes and allowed to clot at controlled room temperature until centrifuged at controlled room temperature at 1300 g for 10 minutes within 60 minutes of collection. The resulting serum was placed in 1 aliquot within 30 minutes of the start of centrifugation in prelabeled 0.50 mL 2D barcoded Matrix tubes, Thermo Cat 3744. All aliquots were flash frozen on dry ice and stored frozen at -60° C. to -90° C.

Serum samples were tested for the presence of anti-drug antibodies (ADA) in an ELISA assay. Test article was immobilized on an assay plate, incubated with serum and washed, and then immune complexes were detected with an anti-Pig IgG reagent having the Fc portion conjugated to horseradish peroxidase for enzymatic detection.

The aqueous humor collection was performed on Day 15 for all animals by a board-certified veterinary ophthalmologist. The aqueous humor collection was performed using conjunctival forceps to fix the globe position while the tip of a 31-gauge needle was inserted bevel up into the sclera immediately posterior to the limbus at approximately a 90-degree angle. The angle of the needle was then shallowed prior to being advanced into the anterior chamber between the iris and cornea. The syringe plunger was slowly withdrawn to aspirate the maximum volume obtainable of up to 50 µL of aqueous humor. The needle was removed, and the episcleral tissues were approximated to the site of insertion and grasped with the conjunctival forceps. An identical sample collection procedure was performed for the contralateral eye. The collected samples were stored in a 1.0 mL glass matrix trakmates 2D barcoded storage tube, and then covered with TPE caps. The samples were frozen in liquid nitrogen and were stored frozen at 60° C. to -90° C. Test article levels in the aqueous humor were determined using a mass-spec based assay.

### A.2. Sample Preparation

PigFab standard calibration curve was performed by spiking different amounts of PigFab into pig aqueous matrix diluted with 25 mM ammonium bicarbonate. Standards/samples were then treated as follows: disulfide bonds from cysteinyl residues were reduced with 10 mM DTT for 1 h at 60° C., and then the thiol groups were alkylated with 55 mM iodoacetamide for 45 min at room temperature in darkness. Standards/samples were then digested by 36 µg/mL trypsin (Sequencing grade Trypsin, V5111, Promega) and incubated overnight at 37° C. Heavy peptide was spiked into both standards and sample solutions after digestion. Linear calibration curves were obtained for 0.5 - 12 µg/mL concentration range.

### A.3. Labeled Peptide

Peptide standard containing heavy isotopic label in R (LLIYSASFLYSGVPSR m/z: 891.98+2) amino acid was purchased (New England Peptide, Gardner, MA, USA). The characterization and concentration data were provided by the manufacturer. The labeled peptide was stored in 1 mL of water at -80° C.

### A.4. Analysis by Mass Spectrometry (MS)

The digest from PigFab was separated on an Acquity UPLC (Waters Corporation, Milford, MA) under gradient elution using an ACQUITY UPLC Peptide CSH C18 Column (130 Å, 1.7 µm, 1 mm × 100 mm). The column was maintained at 50° C. and the auto-sampler tray was maintained at 8° C. The mobile phase was water containing 0.1 % FA (A) and acetonitrile containing 0.1 % FA (B) at a flow rate of 0.04 mL/min. Sample was eluted with a gradient of 2% - 90% B over 2 min, followed by 2 min decreasing to 2% B to re-equilibrate the column. The injection volume was 10 µL.

The Triple Quad 6500 mass spectrometer (Ab Sciex, Framington, MA) was operated in a positive ion multiple reaction monitoring (MRM) mode fitted with an OptiFlow® Turbo V Ion Source. The PigFab precursor (Q1) ion monitored was LLIYSASFLYSGVPSR (m/z: 886.98+2) with declustering potential at 90 V, and the product (Q3) ion monitored was 359.20 m/z with collision energy at 29 eV. Two other product ions were also monitored as qualifiers, 765.39 m/z and 602.33 m/z with collision energy at 37 eV and 30 eV respectively. The MS/MS setting parameters were as follows: ion spray voltage, 4500 V; curtain gas, 30 psi; nebulizer gas (GS1), 25 psi; temperature, 300° C.; and dwell time, 50 ms. A heavy peptide for PigFab was also generated (891.97 m/z) and quantified using the transition 369.204 m/z with collision energy 29 eV.

Sciex Analyst software version 1.7.1 (TripleTOF) was used for data acquisition. Raw data was visualized with PeakView 2.2.

### B. Results

All animals survived until the scheduled termination date. Thus, no unscheduled euthanasia was required. Test-article related eye exam findings included vitreal haze within the region of test article injection and minimal posterior uveitis.

Findings upon ophthalmic exam were as follows:

(1) Group 2 (WT VG1) - Minimal haze was observed within the temporal vitreous was present on Day 1 in 2 of the 6 animals. This resolved by Day 3 and remained absent through study termination. 1 out of 4 animals had minimal posterior uveitis on Day 15, which resolved by Day 17. Minimal posterior uveitis was not considered clinically significant.

(2) Group 3 (pigFab-VG1) - On Day 1, all 6 animals exhibited vitreous haze within the temporal vitreous, at the region of test article injection. This persisted in all 6 animals on Day 3, and on Day 5 the animals showed signs of spread involving the central vitreous in 4/4 animals. From Day 8 to 22, we observed a reduction in the region of vitreous that was affected, and on Day 29, only 2 of the four animals had slight haze within the temporal vitreous. The regional vitreous haze did not appear to be inflammatory in nature but appeared to have a local effect on vitreous consistency.

(3) Group 4 (pigFab-VG1 + 10 kDa HA) - There were no test article-related eye examination findings over the duration of the study.

Intraocular pressure values were within normal limits in all animals at all time points throughout the study.

None of the animals showed anti-drug antibodies (ADA) against the test articles at any time point.

Color fundus photographs and optical coherence tomography images taken on Day 29 illustrated normal vitreal and retinal morphology in all Group 2 and 4 animals. All Group 3 animals had minimal regional vitreous haze on fundus photographs, and minimal posterior vitreal hyperreflectivity on OCT, consistent with eye examination findings.

Electroretinographic analysis was performed in all animals on Day 29. Animal No. 3006 had moderately reduced b-wave amplitude in both eyes at the Scotopic 0.01 light intensity. All other light intensities were within normal limits, suggesting that this animal had a background abnormality affecting dim-light retinal function. Animal No. 2005 and 4003 displayed some asymmetry between eyes, with mild reductions in amplitude OD compared to OS. This was suspected to be related to recording conditions, including non-central eye positioning OD. There were no findings in any animal suggestive of test article effects.

There were no test article-related macroscopic findings in eyes or optic nerves.

All macroscopic observations in test article-treated animals were either background findings in the species or were considered incidental and not test article related. These observations were of low incidence, lacked a clear dose relationship in incidence or severity, and/or had no correlative test article-related microscopic findings.

There were no test article-related microscopic findings in eyes or optic nerves.

All microscopic observations were considered incidental and not test article related. These observations are known background findings for the species, and/or were of similar incidence and severity for control and test article-treated animals.

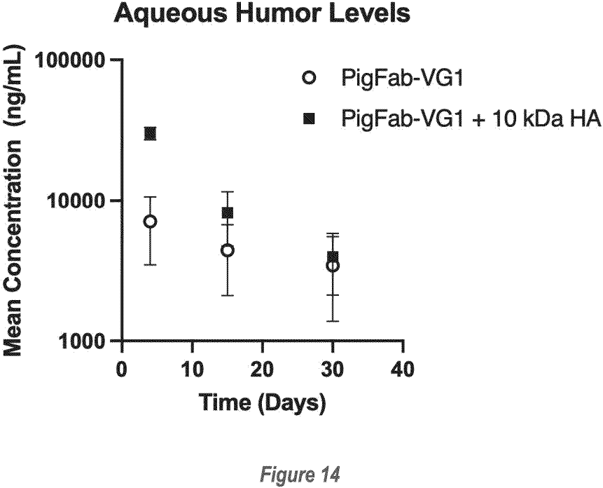

Levels of PigFab-VG1 in aqueous humor samples were determined by mass spectrometry. High aqueous humor levels were obtained and maintained for 30 days following IVT injection of 1.8 mg/eye PigFab-VG1 or PigFab-VG1 pre-complexed with equal mass amount of 10 kDa HA in mini-pig eyes (FIG. 14). These results indicate that measurable levels of test article were present in the mini-pig eye for the duration of the 4-week study. The concentrations at 30 days were at least an order of magnitude higher than measured for an unmodified Fab (FIG. 4A).

In conclusion, administration of WT VG1 (1.13 mg/eye), pigFab-VG1 (1.8 mg/eye) or pigFab-VG1 + 10 kDa HA (1.8 mg/eye) by single IVT injection was well tolerated in Gottingen mini-pigs at each of the respective dose levels. All animals survived to their scheduled terminations, there were no abnormal clinical observations and body weights were not affected. The lack of detectable immune response against the test articles during the study time, and with single injection, allowed direct effects of test article to be assessed. Ophthalmoscopic findings were limited to transient minimal vitreous haze at the test article injection site, which resolved by Day 3 (WT VG-1) and vitreous haze near the test article injection, which improved but did not completely resolve by study termination (pigFab-VG1). There were no ophthalmoscopic findings for pigFab-VG1 + 10 kDa HA and IOP, OCT and ERG results were normal for all animals. There were no test article-related macroscopic or microscopic effects in the eyes or optic nerves.

### Example 15. Efficacy of VPDF-VG1 in Rat Laser-Induced Choroidal Neovascularization (Rat Laser CNV)

Fab-HABDs were studied in an in vivo rat model of laser-induced choroidal neovascularization (rat laser CNV) to test the following assumptions: (1) Fab-HABDs are efficacious in vivo (i.e., Fab-HABDs can inhibit neovascularization) and (2) Fab-HABDs have durability of in vivo efficacy equivalent or superior to the unmodified Fab fragment.

### A. Materials and Methods

Rats received an IVT injection of a protein formulation either one week or three weeks before undergoing laser injury (6 laser burns per eye). One week after setting the laser injury, lesions were analyzed for vascular growth with fluorescence angiography (FA) imaging.

Fab-HABDs were compared to the respective unmodified Fab fragments. For detection of long-lasting efficacy of Fab-HABDs, the dose of unmodified Fab was titrated to a “minimal effect dose” (i.e., only low detectable inhibition of neovascularization in comparison to vehicle within the duration of the rat model) to show longer lasting efficacy for an Fab-HABD at the same dose and duration of the model.

### B. Results

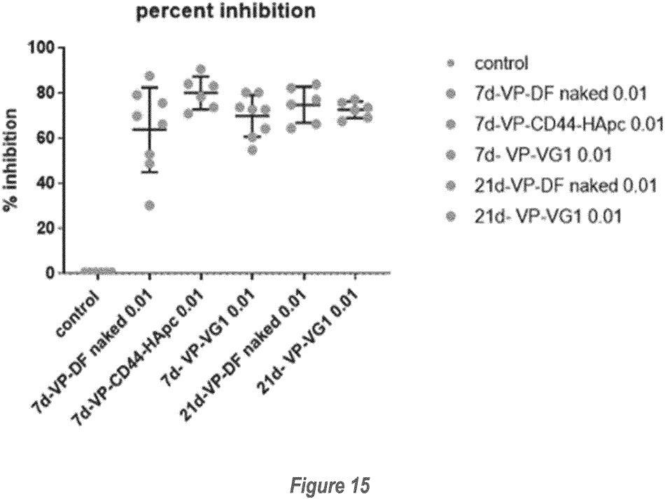

As shown in FIG. 15, VPDF-VG1 was active for inhibition of CNV lesions was administered 7 or 21 days prior to laser treatment. In this study, the durability of effect for VPDF-VG1 was comparable to the unmodified Fab.

### Example 16. Ocular Tolerability of VG1 and VG1 Fab-HABDs in New Zealand White Rabbit

### A. Materials and Methods

The objective of this study was to determine the ocular tolerability of the test articles WT VG1, RabFab-VG1, and RabFab-VG1 pre-formulated with 1:1 (w/w) 10 kDa HA, over a 30-day observation period following a single bilateral IVT injection to male New Zealand White rabbits. The study design was as shown in Table 21.

TABLE 21

Design of ocular tolerability study of VG1 and Fab-HABDs in New Zealand White rabbit

| Group No. | Test Article^{a} | Dose Level (mg/eye) | Dose Volume (µL/eye) | Dose Concentration (mg/mL) | No. of Animals | |
|---|---|---|---|---|---|---|
| | | | | | Day 4 Necropsy | Day 30 Necropsy |
| 1 | Vehicle Control | 0 | 50 | 0 | 3 | - |
| 2 | WT VG1 (SEQ ID NO: 29) | 0.53 | 50 | 10.6 | 2 | 4 |
| 3 | RabFab-VG1 (SEQ ID NOs: 63 and 64 | 0.85 | 50 | 17 | 2 | 4 |
| 4 | RabFab-VG1 + 10 kDa HA | 0.85 | 50 | 17 | 2 | 4 |

No. - Number
- Not applicable
^{a} All test article and vehicle control were administered via bilateral IVT injection once on Day 1.

The following parameters and end points were evaluated in this study: mortality, clinical signs, body weights, food consumption, ophthalmology (i.e., examinations, intraocular pressure measurements, wide-field color fundus imaging, OCT, and ERG), bioanalytical analysis, toxicokinetic parameters, anti-drug antibody evaluations, gross necropsy findings, and histopathologic examinations.

### B. Results

There were no systemic test article-related effects based on assessments of body weight and food consumption. There were also no macroscopic postmortem findings with all tissues considered within normal limits.

The serum of the rabbits was assayed for the presence of anti-drug antibodies (ADA) prior to dosing and at days 8, 15, 22 and 29 of the study. Prior to dosing, 3 out of 6 animals designated for dosing with WT VG1 had measurable serum ADA. Similarly, ⅙ and 0/6 animals designated for treatment with RabFab-VG1 or RabFab-VG1 + 10 kDa HA, respectively, had pre-existing serum ADA against the test article. At Day 8, all of the animals in both the WT VG1 and RabFab-VG1 groups showed ADA against test article in serum, and remained positive for the duration of the study, whereas ⅔ of the animals in the RabFab-VG1 + 10 kDa HA group had serum ADA. At Day 15, all animals were positive for serum ADA that persisted till the end of the study.

Clinical ophthalmoscopic findings were consistent with development of anterior and posterior uveitis in animals treated with WT VG1, RabFab-VG1 as well as RabFab-VG1 + 10 kDa HA, although severity differed between the treatment groups. For example, WT VG1 resulted in moderate anterior and posterior uveitis by Day 22 that included posterior subcapsular cataracts in some eyes. In comparison, most animals treated with RabFab-VG1 displayed mild uveitis at this same time point. In addition, animals treated with RabFab-VG1 + 10 kDa HA exhibited only minimal to mild anterior and posterior uveitis. In each treatment group, signs of uveitis were improved on Day 29 following initiation of systemic anti-inflammatory treatments on Day 18 that also included topical eye treatments for animals dosed with WT VG1. Reduced intraocular pressures were consistent with the active uveitis, while the degree of changes in vitreal haze also corresponded to the differing uveitis severity by group. In these cases, vitreal haze was limited to just faint vitreal opacity in RabFab-VG1 + 10 kDa HA-treated animals, while WT VG1 animals displayed moderate haze and posterior cataracts. Treatment-dependent effect severities were also observed by OCT and ERG with scotopic and photopic amplitude reductions suggestive of severe alterations in retinal function and degeneration in WT VG1-treated eyes.

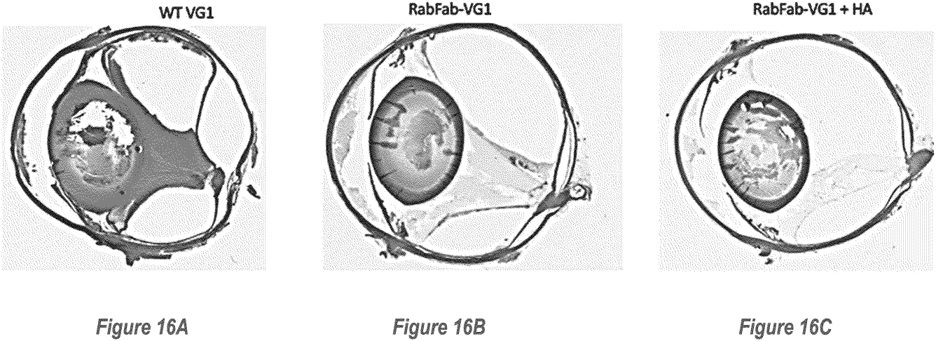

Ocular microscopic effects were also most substantial in eyes treated with WT VG1 (FIG. 16A) compared to the other test materials where RabFab-VG1 effects (FIG. 16B) were less severe and RabFab-VG1 + 10 kDa HA effects (FIG. 16C) were restricted to the vitreous and only minimal to mild in severity. WT VG1-related vitreous inflammation included areas near the optic nerve papilla, detached and variably necrotic retina, and inflammatory cell sheets adjacent to the posterior lens capsule. In addition, the anterior chamber contained homogenous eosinophilic material, consistent with serum proteins. In the retina, WT VG1-related inflammation was characterized by mixed cell types extending into the retinal parenchyma accompanied by minimal-to-marked necrosis with vascular and perivascular inflammation. Reactive Muller cells were observed in the central retina.

Similar to WT VG1, RabFab-VG1-treated eyes displayed minimal-to-moderate diffuse photoreceptor degeneration that was often associated with reactive Muller cells. However, RabFab-VG1 photoreceptor layer degeneration was distinctive from the retinal necrosis associated with WT VG1 because degeneration was selective for only the photoreceptor layer whereas the retina necrosis involved multiple retinal layers. In addition, fibrovascular membranes in the vitreous characterized both the WT VG1- and RabFab-VG1-treated eyes. In these cases, membranes consisted of fibroblasts, numerous new blood vessels, and early collagen deposition. Traction bands were also observed separate from the membranes. In contrast to WT VG1 and RabFab-VG1, RabFab-VG1 + 10 kDa HA-related effects were limited to a minimal to mild mononuclear inflammation that was confined to the vitreous and inner limiting membrane.

In conclusion, single IVT administration of WT VG1, RabFab-VG1 or RabFab-VG1 + 10 kDa HA to New Zealand White rabbits resulted in development of anterior and posterior uveitis, most severe in animals administered WT VG1, moderate with RabFab-VG1 and mild to moderate with RabFab-VG1 + 10 kDa HA. On microscopic examination, in addition to inflammation, there was significant retinal necrosis and retinal degeneration with WT VG1 and RabFab-VG1, respectively, which were correlated with ERG amplitude reductions. There were no signs of active anterior uveitis and observations of only minimal chronic posterior uveitis in the RabFab-VG1 +10 kDa HA eyes at study conclusion. Although the interpretation of these results is confounded by the observation of ADA against all test articles by Day 15, pre-complexation or binding of RabFab-VG1 with 10 kDa HA does appear to improve the tolerability of this Fab-HABD in rabbit eyes.

### Example 17. Brain Retention of Fab-VG1

The capability for HA-binding through VG1 to achieve retention in the brain was tested by intracerebroventricular injection in mice. For these purposes, the non-target binding antibody anti-herpes simplex virus-1 glycoprotein D (anti-gD) was used either as a Fab fragment (anti-gD Fab), intact IgG (anti-gD IgG), or as a fusion protein with VG1 (anti-gD Fab-VG1; BRD).

### A. Materials and Methods

### A.1. Animals

The wild type C57BL/6 mice used in these studies were obtained from The University of Kansas breeding colony. The protocol (AUS 75-15; Approval date: 1/25/21) to use live animals was approved by the Institutional Animal Care and Use Committee (IACUC) at The University of Kansas. All animals were cared for by Animal Care Unit (ACU) personnel and veterinarians under a temperature-controlled environment with a 12-hour dark/light cycle and unlimited access to food and water.

### A.2. Conjugation of Antibodies With IRDye800CW NHS Ester

Antibodies were conjugated with IRDye800 according to the instructions of the manufacturer. Briefly, antibodies in PBS with 10% potassium phosphate buffer, pH 9 (v/v) were reacted with IRDye800 for 2 hours at 25° C. Excess dye was removed using Zeba Spin Desalting Columns with a 7 kDa molecular weight cutoff (Fisher Scientific). The purity of the conjugated antibodies was assessed using SDS-PAGE. An Odyssey CLx NIR scanner was used to scan the SDS-PAGE gel at 800 nm, confirming all excess dye was removed.

### A.3. Intracerebroventricular Injection

Healthy C57BL/6 mice aged 5-10 weeks were anesthetized using 1.5-2% isoflurane and placed into a stereotaxic apparatus (Stoelting Co.). A midline sagittal incision was made to expose the skull of the mice and bregma was identified. A small burr hole was made in the skull 1.0 mm laterally to the right and 0.3 mm anterior to bregma. A 10-µL Hamilton syringe (no. 7762-06) with a 33-gauge removable needle was equipped to the stereotax and was used to 5 µL of antibody solution at a concentration of 1 mg/mL to the lateral ventricle of the mouse at a depth of 2.25 mm. The antibodies were infused at a rate of 1 µL/min. Blood samples (~100 µL) from the submandibular vein were collected immediately prior to euthanasia into chilled plasma collection tubes containing lithium heparin as anticoagulant. Samples were kept on ice until centrifugation for 3 minutes at 10,000×g and the plasma was stored at -80° C. until analysis. Mice were sacrificed after various time points via transcardial perfusion of an ice-cold solution of HBSS with 0.1% Tween-20 while the mice were deeply anesthetized with 4-5% isoflurane. The brain, heart, lungs, liver, spleen, and kidney were collected and kept on ice until analysis.

### A.4. Antibody Organ Quantitation

Isolated organs were weighed and mechanically homogenized in 1 mL of PBS. Standard near infrared fluorescence (NIRF) antibody solutions were created by diluting stock solutions with various amounts of PBS. Calibration curves were then generated for each organ by spiking 10 µL of standard solution into 100 µL of homogenized blank organ into a 96-well plate and scanning the wells using the Odyssey Clx scanner. Fluorescence intensity for each well was plotted over the concentration of antibody per gram of organ to obtain linear curves. Organs from the intracerebroventricularly injected mice were compared to the calibration curves to determine the antibody deposition. Plasma analysis was performed similarly with blank plasma first diluted 5-fold. Then, 100 µL aliquots of the diluted plasma was spiked with 10 µL of antibody standard to generate the standard curve to which the intracerebroventricularly injected mice plasma samples were compared.

### B. Results

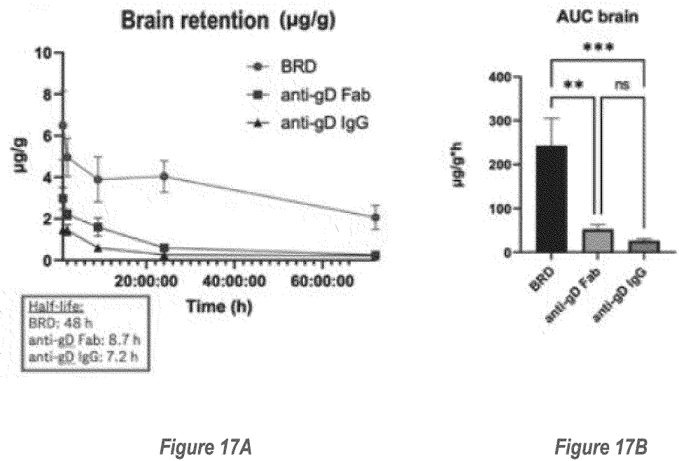

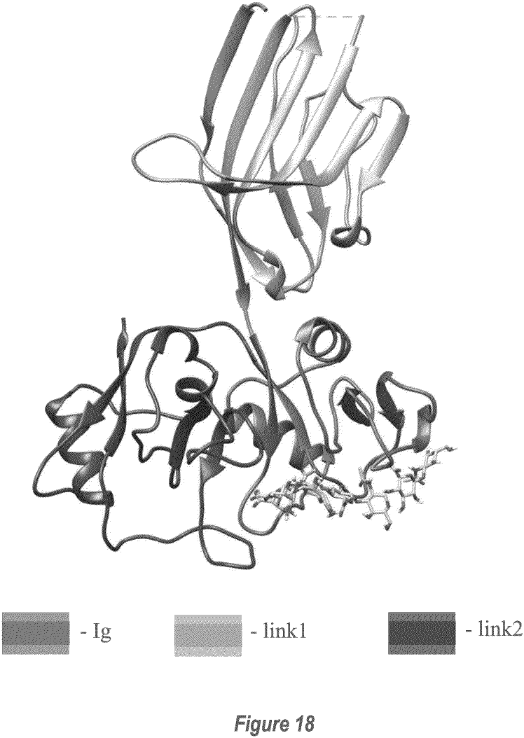

As shown in FIGS. 17, anti-gD Fab-VG1 (BRD; SEQ ID NOS: 121 and 124) persisted in the brain longer and gave a greater exposure level, represented as area-under-the-curve (AUC), than equivalent doses of anti-gD Fab (SEQ ID NOS: 120 and 121) or anti-gD IgG (SEQ ID NOS: 121 and 122). These differences in exposure level are statistically significant with p-value less than 0.01 for comparison of anti-gD Fab-VG1 and anti-gD Fab, and p-value less than 0.001 for comparison of anti-gD Fab-VG1 and anti-gD IgG.

### Example 18. Generation of VG1 Affinity Variants

### A. Materials and Methods

### A.1. Crystallization of WT VG1 and Identification of HA Binding Residues

Commercial crystallization screens (Hampton Research and Qiagen) were used to identify crystallization conditions for WT VG1 in conjugated with HA. The structure of the HA bound VCAN was obtained by soaking crystals with HA6-mer. Crystals were harvested and flash frozen in liquid nitrogen without cryoprotectant. Diffraction data was collected at the Stanford Synchrotron Radiation Lightsource (SSRL) beamLine 12-2 or 14-1 on a Pilatus 6M or Eiger 16M detector (Dectris), respectively. The structures were iteratively refined by model-building in COOT followed by refinement with REFMAC5, BUSTER, or Phenix-Refine. Adams, P.D. et al., Acta Crystallogr. D Biol. Crystallogr., 66(Pt 2):213-221 (2010); Blanc, E. et al., Acta Crystallogr. D Biol. Crystallogr., 60:2210-2221 (2004); Emsley, P. et al., Acta Crystallogr. D Biol. Crystallogr., 66:486-501 (2010); Emsley and Cowtan, Acta Crystallogr. D Biol. Crystallogr., 66:2126-2132 (2004); Murshudov, G.N. et al., Acta Crystallogr. D Biol. Crystallogr., 67:355-367, (2011).

The WT VG1-HA conjugate structure (FIG. 18) was analyzed using either PyMol and/or Chimera and residues interacting with HA were identified based on hydrogen bonding, electrostatic and hydrophobic interaction potentials.

### A.2. Differential Scanning Fluorescence (DSF)

Thermal stability of WT VG1 and single amino acid variants was measured using differential scanning fluorescence (DSF). Briefly, 0.1 mg/mL of purified protein was mixed with Sypro Orange dye in PBS. Each sample was subjected to a temperature gradient from 25° C. to 95° C. in 0.05 °/s increments and increase in fluorescence was monitored at 585 nm. The raw fluorescence units were plotted as negative derivatives using custom excel macros and Tm calculated.

### A.3. VG1 Variants Designed Based on the WT VG1-HA Conjugate Crystal Structure

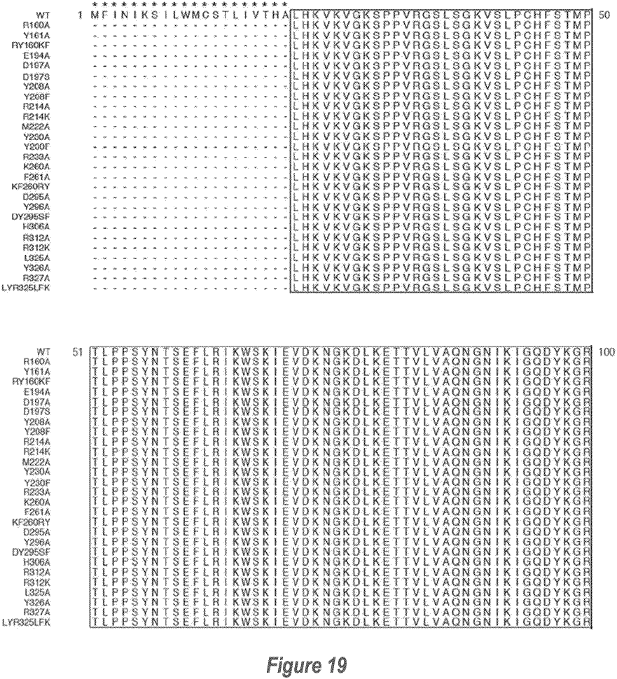

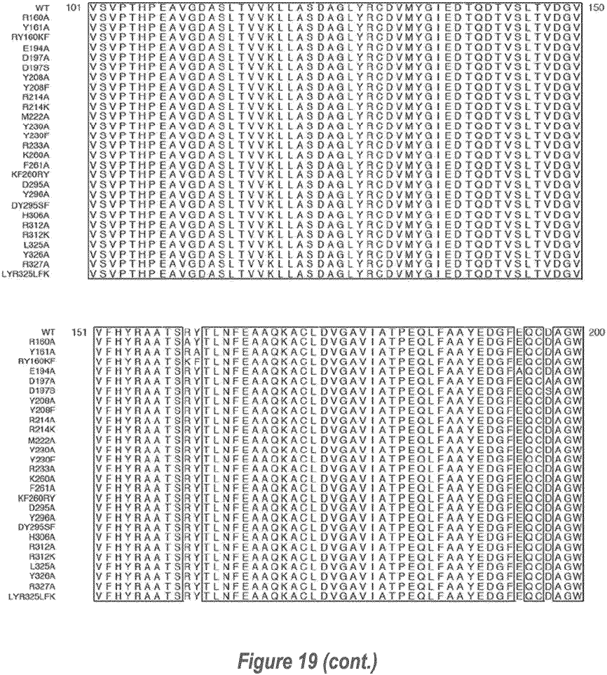

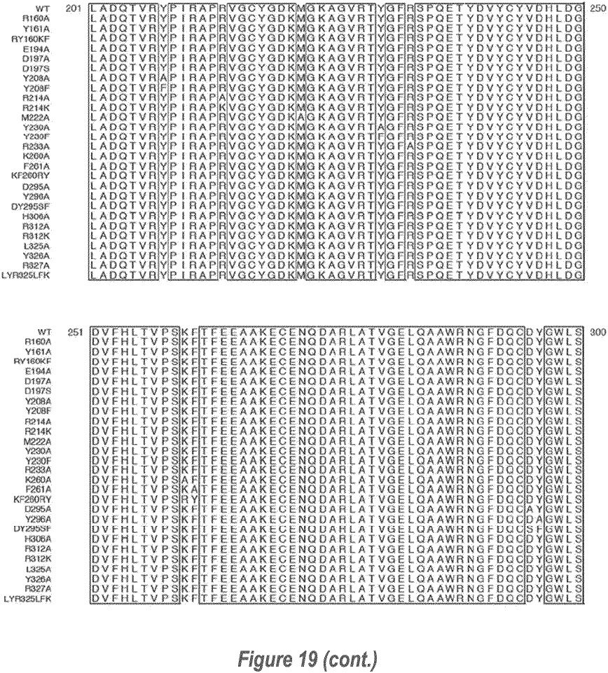

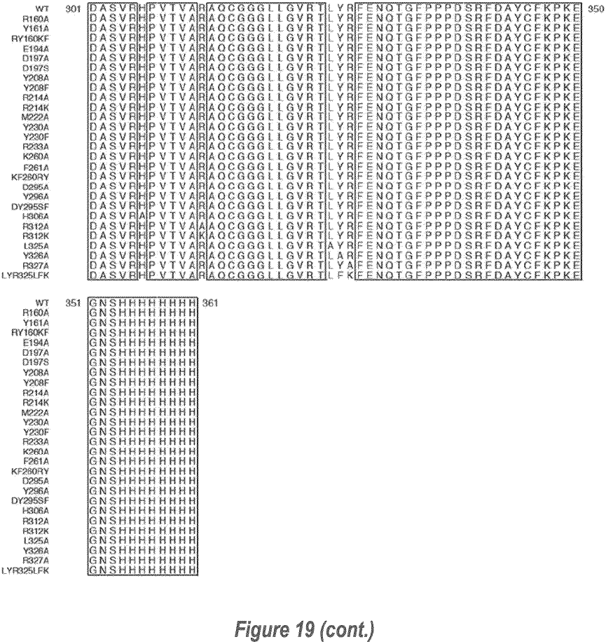

According to the WT VG1-HA conjugate crystal structure (FIG. 18), HA was found to be bound only to the link 1 domain. Thus, modeling was used to predict the link 2 residues that may be involved in HA binding. In order to validate the crystal structure and identify mutants that attenuate HA binding affinity of WT VG1, the residues making crystal contacts were either mutated to alanine, or in some cases, alternate amino acids. Furthermore, some WT VG1 residues that did not make crystal contacts with HA but were important for HA binding in TSG6 (based on sequence alignment between VG1 link domains and TSG-6 link domains; FIG. 8B) were also mutated to alanine. To probe combination effects of mutations, a few double site mutants, for example Lys260 and Phe261 changed to Arg and Tyr, respectively (KF260RY), were produced and tested for HA-binding. Table 22 lists the VG1 variants that were produced as described in Example 10. An amino acid sequence alignment of the VG1 variants produced and tested is shown in FIG. 19.

TABLE 22

Rational mutants produced for VG1

| | | |
|---|---|---|
| VG1 residue* | Mutation | Reason |
| R160 | A | Crystal contact |
| Y161 | A | Crystal contact |
| E194 | A | Corresponding residue important in TGS6 |
| D197 | A | Crystal contact |
| D197 | S | Crystal contact |
| Y208 | A | Crystal contact |
| Y208 | F | Crystal contact |
| R214 | A | Crystal contact |
| R214 | K | Crystal contact |
| M222 | A | Corresponding residue important in TGS6 |
| Y230 | A | Crystal contact |
| Y230 | F | Crystal contact |
| R233 | A | Crystal contact through water molecule |
| K260 | A | Predicted to bind HA |
| F261 | A | Predicted to bind HA |
| D295 | A | Predicted to bind HA |
| Y296 | A | Predicted to bind HA |
| H306 | A | Predicted to bind HA |
| R312 | A | Predicted to bind HA |
| R312 | K | Predicted to bind HA |
| L325 | A | Predicted to bind HA |
| Y326 | A | Predicted to bind HA |
| R327 | A | Predicted to bind HA |

*Single-letter code for amino acids is used

### A.4. Molecular Properties

HA binding was measured by SPR as described in Example 10. The mutants were injected for 120 sec and dissociation monitored for 180 sec.

### B. Results

### B.1. VG1 Variants Have Decreased HA Binding

Mutants R160A, Y161A and D197A displayed attenuated HA binding in the range of 2 to 7 µM. Table 23 shows the measured kₐ (M⁻¹s⁻¹), k_{d} (s⁻¹), and K_{D} (M) for each VG1 variant as measured by SPR.

TABLE 23

HA-binding of point mutants of VG1

| SEQ ID NO | Mutant | kₐ (M⁻¹s⁻¹) | k_{d} (s⁻¹) | K_{D} (M) |
|---|---|---|---|---|
| 29 | WT VG1 | 5.36E+4 | 0.0099 | 1.840E-7 |
| 33 | R160A | 5.92E+04 | 0.127 | 2.14E-06 |
| 34 | Y161A | 2.78E+04 | 0.056 | 2.02E-06 |
| 35 | E194A | No binding | No binding | No binding |
| 36 | D197A | 1.11E+04 | 0.082 | 7.41E-06 |
| 37 | D197S | 6.98E+04 | 0.046 | 6.59E-07 |
| 38 | Y208A | 3.85E+04 | 0.0095 | 2.47E-07 |
| 39 | Y208F | 2.00E+05 | 0.043 | 2.13E-07 |
| 40 | R214A | No binding | No binding | No binding |
| 41 | R214K | 5.25E+04 | 0.0253 | 4.82E-07 |
| 42 | M222A | 2.14E+04 | 0.0091 | 4.25E-07 |
| 43 | Y230A | 4.02E+04 | 0.0080 | 2.00E-07 |
| 44 | Y230F | 1.07E+05 | 0.0386 | 3.60E-07 |
| 45 | R233A | 6.30E+04 | 0.0488 | 7.75E-07 |
| 46 | K260A | 2.26E+04 | 0.0096 | 4.23E-07 |
| 47 | F261A | No binding | No binding | No binding |
| 48 | D295A | 3.22E+05 | 0.2825 | 8.77E-07 |
| 49 | Y296A | 1.24E+05 | 0.0389 | 3.13E-07 |
| 50 | H306A | 3.50E+04 | 0.0114 | 3.25E-07 |
| 51 | R312A | 3.00E+04 | 0.0122 | 4.06E-07 |
| 52 | R312K | 3.63E+04 | 0.0073 | 2.01E-07 |
| 53 | L325A | 4.02E+04 | 0.0083 | 2.07E-07 |
| 54 | Y326A | 3.43E+04 | 0.0084 | 2.46E-07 |
| 55 | R327A | 3.76E+04 | 0.0172 | 4.57E-07 |
| 56 | RY160KF | No binding | No binding | No binding |
| 57 | LYR325LFK | 2.95E+04 | 0.0090 | 3.05E-07 |
| 58 | KF260RY | 5.31E+04 | 0.0086 | 1.62E-07 |
| 59 | DY295SF | 6.53E+04 | 0.0402 | 6.15E-07 |

### B.2. Stability of VG1 Variants

Table 24 shows the VG1 mutants that were produced and the measured Tm (melting temperature; °C) for each mutant. While most mutations either had a slight reduction or no impact on thermal stability as compared to WT VG1, Y208A and H306A displayed a 2.16° C. and 2.81° C. improvement in Tm, respectively.

TABLE 24

Thermal stability measurements of binding site mutants

| SEQ ID NO | Mutant | Tm (°C) |
|---|---|---|
| 29 | WT VG1 | 54.7±0.1 |
| 32 | VGΔIg | 55.8±0.8 |
| 33 | R160A | 55.2 |
| 34 | Y161A | 54.9 |
| 35 | E194A | 54.2 |
| 36 | D197A | 56.0 |
| 37 | D197S | 54.8 |
| 38 | Y208A | 57.0 |
| 39 | Y208F | 55.2 |
| 40 | R214A | 55.5 |
| 41 | R214K | 55.1 |
| 42 | M222A | 55.5 |
| 43 | Y230A | 54.6 |
| 44 | Y230F | 55.6 |
| 45 | R233A | 54.6 |
| 46 | K260A | 52.7 |
| 47 | F261A | 51.3 |
| 48 | D295A | 55.6 |
| 49 | Y296A | 54.8 |
| 50 | H306A | 57.6 |
| 51 | R312A | 54.8 |
| 52 | R312K | 54.0 |
| 53 | L325A | 52.1 |
| 54 | Y326A | 53.8 |
| 55 | R327A | 55.2 |
| 56 | RY160KF | 54.2 |
| 57 | LYR325LFK | 51.9 |
| 58 | KF260RY | 54.0 |
| 59 | DY295SF | 55.3 |

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/-5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A therapeutic molecule comprising:
a. first component capable of binding to a therapeutic target in the eye,
b. one or more second components capable of binding to hyaluronan, wherein the one or more second components are covalently bound to the first component, and
c. optionally, one or more third components comprising hyaluronan,
wherein, if present, the one or more third components are non-covalently bound to the one or more second components.

2. The therapeutic molecule of claim 1, wherein the first component is a protein, a peptide, a receptor or fragment thereof, a ligand to a receptor, a darpin, a nucleic acid, an RNA, a DNA, or an aptamer.

3. The therapeutic molecule of claim 1, wherein the first component is chosen from an antibody, antigen-binding fragment, particularly an antibody fragment, more particularly an antibody fragment lacking at least the Fc domain, especially wherein the fragment is or comprises an (Fab′)₂ fragment, Fab′ fragment, Fab fragment, VhH fragment, scFv fragment, scFv-Fc fragment, and minibody, more especially an Fab fragment.

4. The therapeutic molecule of claim 1, wherein the second component comprises a hyaluronan receptor CD44 (CD44) domain, a brain-specific link protein (BRAL1) domain, a tumor necrosis factor-stimulated gene-6 (TSG-6) domain, a Lymphatic Vessel Endothelial Hyaluronan Receptor-1 (LYVE-1) domain, or a Hyaluronic Acid Binding Protein (HABP) domain, or an Aggrecan G1 (AG1) domain.

5. The therapeutic molecule of claim 1, wherein the conjugate comprises one second component or two second components that are identical to each other.

6. The therapeutic molecule of claim 1, wherein the third component is a hyaluronan, wherein the hyaluronan
a. has a molecular weight
i. chosen from 3 kDa to 60 kDa, from 4 kDa to 30 kDa, from 5 kDa to 20 kDa, or from 400 Da to 200 kDa;
ii. of at least 2, 3, 4, 5, 6, 7, 8, or 9 kDa; or
iii. of at most 60, 50, 40, 30, 25, 20, or 15 kDa;
b. provides a molar excess of binding equivalents to the one or two second components; and
c. has a modification reducing degradation of the hyaluronan in the eye.

7. The therapeutic molecule of claim 1, wherein the second component is capable of binding to hyaluronan with a K_{D} of 10 nM to 10 µM, 5 nM to 8 µM, or 100 nM to 5 µM.

8. The therapeutic molecule of claim 1, wherein a. the first and the second components are comprised in a fusion protein, particularly wherein the one or two of second components are covalently bound to the N-terminus and/or the C-terminus of the first component, more particularly wherein the first component is an antibody or antigen-binding fragment and wherein the one or two second components are covalently bound to a C-terminus of the first component; and/or b. the one or two second components are directly bound to the first component or bound indirectly to the first component via a linker, particularly a linker of at least 4 amino acids and/or at most 50 or at most 25 amino acids, more particularly a linker being (GxS)ₙ or (GxS)nGₘ with G = glycine, S = serine, (x = 3, n = 3, 4, 5 or 6, and m = 0, 1, 2 or 3) or (x = 4, n = 2, 3, 4 or 5 and m = 0, 1, 2 or 3).

9. The therapeutic molecule of claim 1, wherein the therapeutic target is VEGF, C2, C3a, C3b, C5, C5a, HtrA1, IL-33, Factor P, Factor D, EPO, EPOR, IL-1β, IL-17A, IL-10, TNFα, FGFR2, PDGF or ANG2.

10. The therapeutic molecule of claim 1, wherein
a. the first component is an antibody or antigen-binding fragment against VEGF; and/or
b. each of the one or two second components comprise a CD44 domain or a TSG-6 domain; and/or
c. the third component is a hyaluronan of a molecular weight of from 5 kDa to 20 kDa.

11. The therapeutic molecule of claim 1, wherein
a. the first component is an anti-VEGF antibody or antigen-binding fragment, the one or two second components comprise a CD44 domain, and the third component is a hyaluronan of a molecular weight of from 5 kDa to 20 kDa; or
b. the first component is an anti-VEGF antibody or antigen-binding fragment, the one or two second components comprise a TSG-6 domain, and the third component is a hyaluronan of a molecular weight of from 5 kDa to 20 kDa.

12. The therapeutic molecule of claim 1, wherein
a. the first component comprises
i. the VH domain of SEQ ID NO: 97, 99, 105, 109, or 114; and
ii. the VL domain of SEQ ID NO: 98, 100, 106, 110, or 115; and
b. the second component comprises SEQ ID NO: 2.

13. The therapeutic molecule of claim 1, wherein
a. the first component comprises
i. the VH domain of SEQ ID NO: 97, 99, 105, 109, or 114; and
ii. the VL domain of SEQ ID NO: 98, 100, 106, 110, or 115; and
b. the second component comprises SEQ ID NO: 4.

14-23. (canceled)

24. The therapeutic molecule of claim 1, wherein the first component further comprises a cysteine knot peptide.

25. The therapeutic molecule of claim 24, wherein the cysteine knot peptide is at least 90%, 91%, 92%, 93%, 94%, 95%, 96%, 97%, 98%, 99%, or 100% identity with SEQ ID NO: 92.

26. The therapeutic molecule of claim 24, wherein the amino acid sequence comprises at least 90%, 91%, 92%, 93%, 94%, 95%, 96%, 97%, 98%, 99%, or 100% identity with SEQ ID NO: 93 or SEQ ID NO: 94.

27. A pharmaceutical composition comprising the therapeutic molecule of claim 1 and a pharmaceutically acceptable excipient, diluent or carrier.

28. A method of treating an eye disease or a brain disease, comprising administering to a subject a the-composition comprising the therapeutic molecule of claim 1 and optionally a pharmaceutically acceptable excipient, diluent or carrier.

29. The composition use method of claim 28, wherein the composition is formulated for intraocular delivery, particularly intravitreal injection.

30. The method of claim 28, wherein the eye disease is age-related macular degeneration (AMD), particularly wet AMD or neovascular AMD, diabetic macular edema (DME), diabetic retinopathy (DR), particularly proliferative DR or non-proliferative DR, retinal vein occlusion (RVO) or geographic atrophy (GA).

31. A method of delivery for a therapeutic molecule targeted to a tissue in a patient comprising administering the therapeutic molecule of claim 1 to the patient and allowing the therapeutic molecule to provide long-acting delivery of the first component to the target tissue.

32. A method of delivery for a therapeutic molecule targeted to a tissue in a patient comprising administering the pharmaceutical composition of claim 27 to the patient and allowing the therapeutic molecule to provide long-acting delivery of the first component to the target tissue.

33. The therapeutic molecule of claim 1, wherein the second component does not comprise a Versican G1 (VG1) domain.


SEQUENCE LISTING (124 sequences)

SEQ ID NO: 1 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 2 - protein, 150 aa, synthetic construct
  REGION 1..150: note = Synthetic polypeptide
AQIDLNITCRFAGVFHVEKNGRYSISRTEAADLCKAFNSTLPTMAQMEKALSIGFETCRYGFIEGHVVIPRIHPNSICAANNTGVYILTYNTSQYDTYCFNASAPPEEDCTSVTDLPNAFDGPITITIVNRDGTRYVQKGEYRTNPEDIY

SEQ ID NO: 3 - protein, 277 aa, synthetic construct
  REGION 1..277: note = Synthetic polypeptide
MIILIYLFLLLWEDTQGWGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 4 - protein, 104 aa, synthetic construct
  REGION 1..104: note = Synthetic polypeptide
GVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKHHHHHH

SEQ ID NO: 5 - protein, 384 aa, synthetic construct
  REGION 1..384: note = Synthetic polypeptide
DLQLVESGGGLVKPGGSLRLSCAADGWWFGYTDMSWVRQAPGKGLEWVGSISYKGGSTYYNTKFIGRFTISRDDDTNTLYLQMNSLRAEDTAVYYCARDDGYFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSAQIDLNITCRFAGVFHVEKNGRYSISRTEAADLCKAFNSTLPTMAQMEKALSIGFETCRYGFIEGHVVIPRIHPNSICAANNTGVYILTYNTSQYDTYCFNASAPPEEDCTSVTDLPNAFDGPITITIVNRDGTRYVQKGEYRTNPEDIY

SEQ ID NO: 6 - protein, 213 aa, synthetic construct
  REGION 1..213: note = Synthetic polypeptide
AIYMHQEPSSLSASVGDRVTITCHGSYWLSNYLAWYQQKPGKAPKLLIYDGKEREHGVPSRFSGSGSHEDYTLTISSLQPEDFATYYCQQYRYHPYTFGHGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 7 - protein, 384 aa, synthetic construct; identical to SEQ ID NO: 5
  REGION 1..384: note = Synthetic polypeptide

SEQ ID NO: 8 - protein, 379 aa, synthetic construct
  REGION 1..379: note = Synthetic polypeptide
AIYMHQEPSSLSASVGDRVTITCHGSYWLSNYLAWYQQKPGKAPKLLIYDGKEREHGVPSRFSGSGSHEDYTLTISSLQPEDFATYYCQQYRYHPYTFGHGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSAQIDLNITCRFAGVFHVEKNGRYSISRTEAADLCKAFNSTLPTMAQMEKALSIGFETCRYGFIEGHVVIPRIHPNSICAANNTGVYILTYNTSQYDTYCFNASAPPEEDCTSVTDLPNAFDGPITITIVNRDGTRYVQKGEYRTNPEDIY

SEQ ID NO: 9 - protein, 393 aa, synthetic construct
  REGION 1..393: note = Synthetic polypeptide
QVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSAQIDLNITCRFAGVFHVEKNGRYSISRTEAADLCKAFNSTLPTMAQMEKALSIGFETCRYGFIEGHVVIPRIHPNSICAANNTGVYILTYNTSQYDTYCFNASAPPEEDCTSVTDLPNAFDGPITITIVNRDGTRYVQKGEYRTNPEDIY

SEQ ID NO: 10 - protein, 214 aa, synthetic construct
  REGION 1..214: note = Synthetic polypeptide
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 393 aa, synthetic construct; identical to SEQ ID NO: 9
  REGION 1..393: note = Synthetic polypeptide

SEQ ID NO: 12 - protein, 379 aa, synthetic construct
  REGION 1..379: note = Synthetic polypeptide
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSAQIDLNITCRFAGVFHVEKNGRYSISRTEAADLCKAFNSTLPTMAQMEKALSIGFETCRYGFIEGHVVIPRIHPNSICAANNTGVYILTYNTSQYDTYCFNASAPPEEDCTSVTDLPNAFDGPITITIVNRDGTRYVQKGEYRTNPEDIY

SEQ ID NO: 13 - protein, 329 aa, synthetic construct
  REGION 1..329: note = Synthetic polypeptide
QSLEESGGRLVTPGTPLTLTCTVSGFTISSYHMSWVRQAPGKGLEWIGIMRNTANIYYASWAKGRFTISKTSPTTVDLKMTSLTTEDTATYFCARGRPGDGALSLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTGGGGSGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKHHHHHH

SEQ ID NO: 14 - protein, 215 aa, synthetic construct
  REGION 1..215: note = Synthetic polypeptide
ADVVMTQTPASVSAAVGGTVTIKCQASQSIGTALAWYQQKPGQPPKLLIYRTSTLESGVPSRFKGSGSGTDFTLTISDLECADAATYYCQSAYVSGGNIYTFGGGTEVVVKGDPVAPTVLIFPPAADQVATGTVTIVCVANKYFPDVTVTWEVDGTTQTTGIENSKTPQNSADCTYNLSSTLTLTSTQYNGHKEYTCKVTQGTTSVVQSFNRGDC

SEQ ID NO: 15 - protein, 329 aa, synthetic construct; identical to SEQ ID NO: 13
  REGION 1..329: note = Synthetic polypeptide

SEQ ID NO: 16 - protein, 326 aa, synthetic construct
  REGION 1..326: note = Synthetic polypeptide
ADVVMTQTPASVSAAVGGTVTIKCQASQSIGTALAWYQQKPGQPPKLLIYRTSTLESGVPSRFKGSGSGTDFTLTISDLECADAATYYCQSAYVSGGNIYTFGGGTEVVVKGDPVAPTVLIFPPAADQVATGTVTIVCVANKYFPDVTVTWEVDGTTQTTGIENSKTPQNSADCTYNLSSTLTLTSTQYNGHKEYTCKVTQGTTSVVQSFNRGDCGGGGSGVYHREARSGKYKLTYAEAKAVCEFEGGRLATYKQLEAARKIGFHVCAAGWMAKGRVGYPIVKPGSNCGFGKTGIIDYGIRLNRSERWDAYCYNPHAKDYKDDDDK

SEQ ID NO: 17 - protein, 337 aa, synthetic construct
  REGION 1..337: note = Synthetic polypeptide
EVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKHHHHHH

SEQ ID NO: 18 - protein, 214 aa, synthetic construct
  REGION 1..214: note = Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQGYGNPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 337 aa, synthetic construct; identical to SEQ ID NO: 17
  REGION 1..337: note = Synthetic polypeptide

SEQ ID NO: 20 - protein, 325 aa, synthetic construct
  REGION 1..325: note = Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQGYGNPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKDYKDDDDK

SEQ ID NO: 21 - protein, 325 aa, synthetic construct
  REGION 1..325: note = Synthetic polypeptide
EVQLVESGGGLVQPGGSLRLSCTASGFSLTNYYYMTWVRQAPGKGLEWVGFIDPQNDPYYATWAKGRFTISRDNSKNTLYLQMNSLRAEDTAVYYCAGGNHNSGWGLNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAISGKYYLTYAEAKAVCEFEGGHLATYKQLLAAQKIGFHVCAAGWMAKGRVGYPIVKPGPNCGFGKTGIIDYGIRLNRSERWDAYCYNPHA

SEQ ID NO: 22 - protein, 218 aa, synthetic construct
  REGION 1..218: note = Synthetic polypeptide
EIVMTQSPSTLSASVGDRVIITCQASQKIHSWLAWYQQKPGKAPKLLIYQASKLAKGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 327 aa, synthetic construct
  REGION 1..327: note = Synthetic polypeptide
DLQLVESGGGLVKPGGSLRLSCAADGWWFGYTDMSWVRQAPGKGLEWVGSISYKGGSTYYNTKFIGRFTISRDDDTNTLYLQMNSLRAEDTAVYYCARDDGYFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGVYHREAISGKYYLTYAEAKAVCEFEGGHLATYKQLLAAQKIGFHVCAAGWMAKGRVGYPIVKPGPNCGFGKTGIIDYGIRLNRSERWDAYCYNPHAK

SEQ ID NO: 24 - protein, 214 aa, synthetic construct
  REGION 1..214: note = Synthetic polypeptide
AIYMHQEPSSLSASVGDRVTITCHGSYWLSNYLAWYQQKPGKAPKLLIYDGKEREHGVPSRFSGSGSHEDYTLTISSLQPEDFATYYCQQYRYHPYTFGHGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 384 aa, synthetic construct
  REGION 1..384: note = Synthetic polypeptide
DLQLVESGGGLVKPGGSLRLSCAADGWWFGYTDMSWVRQAPGKGLEWVGSISYKGGSTYYNTKFIGRFTISRDDDTNTLYLQMNSLRAEDTAVYYCARDDGYFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSAQIDLNITCRFAGVFHVEKNGRSSISRTEAADLCKAFNSTLPTMAQMEKALSIGFETCRYGFIEGHVVIPRIHPNSICAANNTGVYILTYNTSQYDTYCFNASAPPEEDCTSVTDLPNAFDGPITITIVNRDGTRYVQKGEYRTNPEDIY

SEQ ID NO: 26 - protein, 379 aa, synthetic construct
  REGION 1..379: note = Synthetic polypeptide
AIYMHQEPSSLSASVGDRVTITCHGSYWLSNYLAWYQQKPGKAPKLLIYDGKEREHGVPSRFSGSGSHEDYTLTISSLQPEDFATYYCQQYRYHPYTFGHGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSAQIDLNITCRFAGVFHVEKNGRSSISRTEAADLCKAFNSTLPTMAQMEKALSIGFETCRYGFIEGHVVIPRIHPNSICAANNTGVYILTYNTSQYDTYCFNASAPPEEDCTSVTDLPNAFDGPITITIVNRDGTRYVQKGEYRTNPEDIY

SEQ ID NO: 27 - protein, 5 aa, synthetic construct
  REGION 1..5: note = Synthetic polypeptide
GGGGS

SEQ ID NO: 28 - protein, 15 aa, synthetic construct
  REGION 1..15: note = Synthetic polypeptide
GGGGSGGGGSGGGGS

SEQ ID NO: 29 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 30 - protein, 108 aa, synthetic construct
  REGION 1..108: note = Synthetic polypeptide
GSGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHHHHHHHH

SEQ ID NO: 31 - protein, 112 aa, synthetic construct
  REGION 1..112: note = Synthetic polypeptide
GDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 32 - protein, 212 aa, synthetic construct
  REGION 1..212: note = Synthetic polypeptide
VVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 33 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSAYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 34 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRATLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 35 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFAQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 36 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCAAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 37 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCSAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 38 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRAPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 39 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRFPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 40 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPAVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 41 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPKVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 42 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKAGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 43 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTAGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 44 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTFGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 45 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFASPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 46 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSAFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 47 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKATFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 48 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCAYGWLSDASVRHPVTVARAQCGGGLLGVRTLYRFENQTGFPPPDSRFDAYCFKPKEGNSHHHHHHHH

SEQ ID NO: 49 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDAGWLSDASVRHPVTVARAQCGGGLLGVRTLYRFENQTGFPPPDSRFDAYCFKPKEGNSHHHHHHHH

SEQ ID NO: 50 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRAPVTVARAQCGGGLLGVRTLYRFENQTGFPPPDSRFDAYCFKPKEGNSHHHHHHHH

SEQ ID NO: 51 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVAAAQCGGGLLGVRTLYRFENQTGFPPPDSRFDAYCFKPKEGNSHHHHHHHH

SEQ ID NO: 52 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVAKAQCGGGLLGVRTLYRFENQTGFPPPDSRFDAYCFKPKEGNSHHHHHHHH

SEQ ID NO: 53 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTAYRFENQTGFPPPDSRFDAYCFKPKEGNSHHHHHHHH

SEQ ID NO: 54 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLARFENQTGFPPPDSRFDAYCFKPKEGNSHHHHHHHH

SEQ ID NO: 55 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYAFENQTGFPPPDSRFDAYCFKPKEGNSHHHHHHHH

SEQ ID NO: 56 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSKFTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 57 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLFKFENQTGFPPPDSRFDAYCFKPKEGNSHHHHHHHH

SEQ ID NO: 58 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSRYTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 59 - protein, 341 aa, synthetic construct
  REGION 1..341: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCSFGWLSDASVRHPVTVARAQCGGGLLGVRTLYRFENQTGFPPPDSRFDAYCFKPKEGNSHHHHHHHH

SEQ ID NO: 60 - protein, 330 aa, synthetic construct
  REGION 1..330: note = Synthetic polypeptide
  SITE 177: note = misc_feature - X is Asp or Ser
  SITE 188: note = misc_feature - X is any amino acid
  SITE 194: note = misc_feature - X is Arg or Ser
  SITE 202: note = misc_feature - X is any amino acid
  SITE 210: note = misc_feature - X is any amino acid
  SITE 240: note = misc_feature - X is any amino acid
  SITE 241: note = misc_feature - X is F or Y
  SITE 275: note = misc_feature - X is Asp or Ser
  SITE 276: note = misc_feature - X is any amino acid
  SITE 286: note = misc_feature - X is any amino acid
  SITE 292: note = misc_feature - X is any amino acid
  REGION 305..307: note = misc_feature - X is any amino acid
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCXAGWLADQTVRXPIRAPXVGCYGDKXGKAGVRTXGFRSPQETYDVYCYVDHLDGDVFHLTVPSXXTFEEAAKECENQDARLATVGELQAAWRNGFDQCXXGWLSDASVRXPVTVAXAQCGGGLLGVRTXXXFENQTGFPPPDSRFDAYCFKPKE

SEQ ID NO: 61 - protein, 215 aa, synthetic construct; identical to SEQ ID NO: 14
  REGION 1..215: note = Synthetic polypeptide

SEQ ID NO: 62 - protein, 220 aa, synthetic construct
  REGION 1..220: note = Synthetic polypeptide
QSLEESGGRLVTPGTPLTLTCTVSGFTISSYHMSWVRQAPGKGLEWIGIMRNTANIYYASWAKGRFTISKTSPTTVDLKMTSLTTEDTATYFCARGRPGDGALSLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPT

SEQ ID NO: 63 - protein, 215 aa, synthetic construct; identical to SEQ ID NO: 14
  REGION 1..215: note = Synthetic polypeptide

SEQ ID NO: 64 - protein, 566 aa, synthetic construct
  REGION 1..566: note = Synthetic polypeptide
QSLEESGGRLVTPGTPLTLTCTVSGFTISSYHMSWVRQAPGKGLEWIGIMRNTANIYYASWAKGRFTISKTSPTTVDLKMTSLTTEDTATYFCARGRPGDGALSLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTGGGGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 65 - protein, 216 aa, synthetic construct
  REGION 1..216: note = Synthetic polypeptide
AIQLTQSPASLAASLGDTVSITCRASQDVSTAVAWYQQQAGKAPKLLIYSASFLYSGVPSRFKGSGSGTDFTLTISGLQAEDVATYYCQQGYGNPFTFGQGTKLELKRADAKPSVFIFPPSKEQLETQTVSVVCLLNSFFPREVNVKWKVDGVVQSSGILDSVTEQDSKDSTYSLSSTLSLPTSQYLSHNLYSCEVTHKTLASPLVKSFSRNECEA

SEQ ID NO: 66 - protein, 558 aa, synthetic construct
  REGION 1..558: note = Synthetic polypeptide
EEKLVESGGGLVQPGGSLRLSCVGSGFTISDYWIHWVRQAPGKGLEWLAGITPAGGYTYYADSVKGRFTISSDNSQNTAYLQMNSLRTEDTARYYCARFVFFLPYAMDYWGPGVEVVVSSAPKTAPSVYPLAPCSRDTSGPNVALGCLASSYFPEPVTVTWNSGALSSGVHTFPSVLQPSGLYSLSSMVTVPASSLSSKSYTCNVNHPATTTKVDKRVGTKTKGGGGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKE

SEQ ID NO: 67 - protein, 214 aa, synthetic construct
  REGION 1..214: note = Synthetic polypeptide
DIQMTQSPSSLSASVGDRVTITCRASQDVSTAVAWYQQKPGKAPKLLIYSASFLYSGVPSRFSGSGSGTDFTLTISSLQPEDAATYYCQQGYGAPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 563 aa, synthetic construct
  REGION 1..563: note = Synthetic polypeptide
EVQLVESGGGLVQPGGSLRLSCAASGFTISDYWIHWVRQAPGKGLEWVAGITPAGGYTRYADSVKGRFTISADTSKNTAYLQMRSLRAEDTAVYYCARFVFFLPYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKE

SEQ ID NO: 69 - protein, 214 aa, synthetic construct; identical to SEQ ID NO: 24
  REGION 1..214: note = Synthetic polypeptide

SEQ ID NO: 70 - protein, 559 aa, synthetic construct
  REGION 1..559: note = Synthetic polypeptide
DLQLVESGGGLVKPGGSLRLSCAADGWWFGYTDMSWVRQAPGKGLEWVGSISYKGGSTYYNTKFIGRFTISRDDDTNTLYLQMNSLRAEDTAVYYCARDDGYFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKE

SEQ ID NO: 71 - protein, 566 aa, synthetic construct
  REGION 1..566: note = Synthetic polypeptide
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKRKCLIPFGNSVTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 214 aa, synthetic construct; identical to SEQ ID NO: 24
  REGION 1..214: note = Synthetic polypeptide

SEQ ID NO: 73 - protein, 431 aa, synthetic construct
  REGION 1..431: note = Synthetic polypeptide
DLQLVESGGGLVKPGGSLRLSCAADGWWFGYTDMSWVRQAPGKGLEWVGSISYKGGSTYYNTKFIGRFTISRDDDTNTLYLQMNSLRAEDTAVYYCARDDGYFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKE

SEQ ID NO: 74 - protein, 214 aa, synthetic construct
  REGION 1..214: note = Synthetic polypeptide
DIQMTQSPSSLSASVGDRVTITCKASQNVDTDVAWFQQKPGKAPKGLIRSASSRYSGVPSRFSGSGSGTDFTLTISSLQPEDFATYYCQQYNNYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 558 aa, synthetic construct
  REGION 1..558: note = Synthetic polypeptide
EVQLVQSGAEVKKPGASVKVSCKASGYTFTSYYMYWVRQAPGQGLEWIGEINPTSGGTNFNEKFKSRATLTVDTSTSTAYLELSSLRSEDTAVYYCAREGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKE

SEQ ID NO: 76 - protein, 214 aa, synthetic construct
  REGION 1..214: note = Synthetic polypeptide
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 565 aa, synthetic construct
  REGION 1..565: note = Synthetic polypeptide
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKE

SEQ ID NO: 78 - protein, 379 aa, synthetic construct
  REGION 1..379: note = Synthetic polypeptide
GCPRILMRCKQDSDCLAGCVCGPNGFCGGSGSGSGSGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 79 - protein, 385 aa, synthetic construct
  REGION 1..385: note = Synthetic polypeptide
GCPRILMRCKQDSDCLAGCVCGPNGFCGENLYFQGSGSGSGSGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 80 - protein, 412 aa, synthetic construct
  REGION 1..412: note = Synthetic polypeptide
MGGTAARLGAVILFVVIVGLHGVRHHHHHHHHGENLYFQGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSGSGSGSGSGSGCPRILMRCKQDSDCLAGCVCGPNGFCG

SEQ ID NO: 81 - protein, 419 aa, synthetic construct
  REGION 1..419: note = Synthetic polypeptide
MGGTAARLGAVILFVVIVGLHGVRHHHHHHHHGENLYFQGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSGSGSGSGSGSENLYFQGGCPRILMRCKQDSDCLAGCVCGPNGFCG

SEQ ID NO: 82 - protein, 12 aa, synthetic construct
  REGION 1..12: note = Synthetic polypeptide
RKCLIPFGNSVT

SEQ ID NO: 83 - protein, 4 aa, synthetic construct
  REGION 1..4: note = Synthetic polypeptide
GGGG

SEQ ID NO: 84 - protein, 10 aa, synthetic construct
  REGION 1..10: note = Synthetic polypeptide
GSGSGSGSGS

SEQ ID NO: 85 - protein, 16 aa, synthetic construct
  REGION 1..16: note = Synthetic polypeptide
ENLYFQGSGSGSGSGS

SEQ ID NO: 86 - protein, 201 aa, synthetic construct
  REGION 1..201: note = Synthetic polypeptide
  SITE 48: note = misc_feature - X is Asp or Ser
  SITE 59: note = misc_feature - X is any amino acid
  SITE 65: note = misc_feature - X is Arg or Ser
  SITE 73: note = misc_feature - X is any amino acid
  SITE 81: note = misc_feature - X is any amino acid
  SITE 111: note = misc_feature - X is any amino acid
  SITE 112: note = misc_feature - X is F or Y
  SITE 146: note = misc_feature - X is Asp or Ser
  SITE 147: note = misc_feature - X is any amino acid
  SITE 157: note = misc_feature - X is any amino acid
  SITE 163: note = misc_feature - X is any amino acid
  REGION 176..178: note = misc_feature - X is any amino acid
VVFHYRAATSRYTLNFEAAQKACLDVGAVIATPEQLFAAYEDGFEQCXAGWLADQTVRXPIRAPXVGCYGDKXGKAGVRTXGFRSPQETYDVYCYVDHLDGDVFHLTVPSXXTFEEAAKECENQDARLATVGELQAAWRNGFDQCXXGWLSDASVRXPVTVAXAQCGGGLLGVRTXXXFENQTGFPPPDSRFDAYCFKPKE

SEQ ID NO: 87 - protein, 24 aa, synthetic construct
  REGION 1..24: note = Synthetic polypeptide
  REGION 13..24: note = misc_feature - Residues may be present or absent.
GGGSGGGSGGGSGGGSGGGSGGGS

SEQ ID NO: 88 - protein, 25 aa, synthetic construct
  REGION 1..25: note = Synthetic polypeptide
  REGION 11..25: note = misc_feature - residues may be absent
GGGGSGGGGSGGGGSGGGGSGGGGS

SEQ ID NO: 89 - protein, 27 aa, synthetic construct
  REGION 1..27: note = Synthetic polypeptide
  REGION 13..27: note = misc_feature - residues may be absent
GGGSGGGSGGGSGGGSGGGSGGGSGGG

SEQ ID NO: 90 - protein, 28 aa, synthetic construct
  REGION 1..28: note = Synthetic polypeptide
  REGION 11..28: note = misc_feature - residues may be absent
GGGGSGGGGSGGGGSGGGGSGGGGSGGG

SEQ ID NO: 91 - protein, 4 aa, synthetic construct
  REGION 1..4: note = Synthetic polypeptide
GGGS

SEQ ID NO: 92 - protein, 30 aa, synthetic construct
  REGION 1..30: note = Synthetic polypeptide
GCNIMLPYWGCGRDFECMEQCICQYYQSCG

SEQ ID NO: 93 - protein, 391 aa, synthetic construct
  REGION 1..391: note = Synthetic polypeptide
GCNIMLPYWGCGRDFECMEQCICQYYQSCGGSGSGSGSGSGSGSGSGSGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSHHHHHHHH

SEQ ID NO: 94 - protein, 391 aa, synthetic construct
  REGION 1..391: note = Synthetic polypeptide
HHHHHHHHLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEGNSGSGSGSGSGSGSGSGSGSGSGCNIMLPYWGCGRDFECMEQCICQYYQSCG

SEQ ID NO: 95 - protein, 20 aa, synthetic construct
  REGION 1..20: note = Synthetic polypeptide
GSGSGSGSGSGSGSGSGSGS

SEQ ID NO: 96 - protein, 150 aa, synthetic construct
  REGION 1..150: note = Synthetic polypeptide
AQIDLNITCRFAGVFHVEKNGRSSISRTEAADLCKAFNSTLPTMAQMEKALSIGFETCRYGFIEGHVVIPRIHPNSICAANNTGVYILTYNTSQYDTYCFNASAPPEEDCTSVTDLPNAFDGPITITIVNRDGTRYVQKGEYRTNPEDIY

SEQ ID NO: 97 - protein, 120 aa, synthetic construct
  REGION 1..120: note = Synthetic polypeptide
EEKLVESGGGLVQPGGSLRLSCVGSGFTISDYWIHWVRQAPGKGLEWLAGITPAGGYTYYADSVKGRFTISSDNSQNTAYLQMNSLRTEDTARYYCARFVFFLPYAMDYWGPGVEVVVSS

SEQ ID NO: 98 - protein, 107 aa, synthetic construct
  REGION 1..107: note = Synthetic polypeptide
AIQLTQSPASLAASLGDTVSITCRASQDVSTAVAWYQQQAGKAPKLLIYSASFLYSGVPSRFKGSGSGTDFTLTISGLQAEDVATYYCQQGYGNPFTFGQGTKLELK

SEQ ID NO: 99 - protein, 116 aa, synthetic construct
  REGION 1..116: note = Synthetic polypeptide
DLQLVESGGGLVKPGGSLRLSCAADGWWFGYTDMSWVRQAPGKGLEWVGSISYKGGSTYYNTKFIGRFTISRDDDTNTLYLQMNSLRAEDTAVYYCARDDGYFDTWGQGTLVTVSS

SEQ ID NO: 100 - protein, 107 aa, synthetic construct
  REGION 1..107: note = Synthetic polypeptide
AIYMHQEPSSLSASVGDRVTITCHGSYWLSNYLAWYQQKPGKAPKLLIYDGKEREHGVPSRFSGSGSHEDYTLTISSLQPEDFATYYCQQYRYHPYTFGHGTKVEIK

SEQ ID NO: 101 - protein, 224 aa, synthetic construct
  REGION 1..224: note = Synthetic polypeptide
DLQLVESGGGLVKPGGSLRLSCAADGWWFGYTDMSWVRQAPGKGLEWVGSISYKGGSTYYNTKFIGRFTISRDDDTNTLYLQMNSLRAEDTAVYYCARDDGYFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 102 - protein, 214 aa, synthetic construct; identical to SEQ ID NO: 24
  REGION 1..214: note = Synthetic polypeptide

SEQ ID NO: 103 - protein, 228 aa, synthetic construct
  REGION 1..228: note = Synthetic polypeptide
EVQLVESGGGLVQPGGSLRLSCAASGFTISDYWIHWVRQAPGKGLEWVAGITPAGGYTRYADSVKGRFTISADTSKNTAYLQMRSLRAEDTAVYYCARFVFFLPYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 104 - protein, 214 aa, synthetic construct; identical to SEQ ID NO: 67
  REGION 1..214: note = Synthetic polypeptide

SEQ ID NO: 105 - protein, 120 aa, synthetic construct
  REGION 1..120: note = Synthetic polypeptide
EVQLVESGGGLVQPGGSLRLSCAASGFTISDYWIHWVRQAPGKGLEWVAGITPAGGYTRYADSVKGRFTISADTSKNTAYLQMRSLRAEDTAVYYCARFVFFLPYAMDYWGQGTLVTVSS

SEQ ID NO: 106 - protein, 107 aa, synthetic construct
  REGION 1..107: note = Synthetic polypeptide
DIQMTQSPSSLSASVGDRVTITCRASQDVSTAVAWYQQKPGKAPKLLIYSASFLYSGVPSRFSGSGSGTDFTLTISSLQPEDAATYYCQQGYGAPFTFGQGTKVEIK

SEQ ID NO: 107 - protein, 116 aa, synthetic construct
  REGION 1..116: note = Synthetic polypeptide
QSLEESGGRLVTPGTPLTLTCTVSGFTISSYHMSWVRQAPGKGLEWIGIMRNTANIYYASWAKGRFTISKTSPTTVDLKMTSLTTEDTATYFCARGRPGDGALSLWGQGTLVTVSS

SEQ ID NO: 108 - protein, 111 aa, synthetic construct
  REGION 1..111: note = Synthetic polypeptide
ADVVMTQTPASVSAAVGGTVTIKCQASQSIGTALAWYQQKPGQPPKLLIYRTSTLESGVPSRFKGSGSGTDFTLTISDLECADAATYYCQSAYVSGGNIYTFGGGTEVVVK

SEQ ID NO: 109 - protein, 120 aa, synthetic construct
  REGION 1..120: note = Synthetic polypeptide
EVQLVESGGGLVQPGGSLRLSCTASGFSLTNYYYMTWVRQAPGKGLEWVGFIDPQNDPYYATWAKGRFTISRDNSKNTLYLQMNSLRAEDTAVYYCAGGNHNSGWGLNIWGQGTLVTVSS

SEQ ID NO: 110 - protein, 110 aa, synthetic construct
  REGION 1..110: note = Synthetic polypeptide
EIVMTQSPSTLSASVGDRVIITCQASQKIHSWLAWYQQKPGKAPKLLIYQASKLAKGVPSRFSGSGSGAEFTLTISSLQPDDFATYYCQNVYLASTNGANFGQGTKLTVL

SEQ ID NO: 111 - protein, 115 aa, synthetic construct
  REGION 1..115: note = Synthetic polypeptide
EVQLVQSGAEVKKPGASVKVSCKASGYTFTSYYMYWVRQAPGQGLEWIGEINPTSGGTNFNEKFKSRATLTVDTSTSTAYLELSSLRSEDTAVYYCAREGGFAYWGQGTLVTVSS

SEQ ID NO: 112 - protein, 107 aa, synthetic construct
  REGION 1..107: note = Synthetic polypeptide
DIQMTQSPSSLSASVGDRVTITCKASQNVDTDVAWFQQKPGKAPKGLIRSASSRYSGVPSRFSGSGSGTDFTLTISSLQPEDFATYYCQQYNNYPLTFGQGTKVEIK

SEQ ID NO: 113 - protein, 97 aa, synthetic construct
  REGION 1..97: note = Synthetic polypeptide
GVYHREAISGKYYLTYAEAKAVCEFEGGHLATYKQLLAAQKIGFHVCAAGWMAKGRVGYPIVKPGPNCGFGKTGIIDYGIRLNRSERWDAYCYNPHA

SEQ ID NO: 114 - protein, 123 aa, synthetic construct [sequence omitted]

SEQ ID NO: 115 - protein, 107 aa, synthetic construct [sequence omitted]

SEQ ID NO: 116 - protein, 119 aa, synthetic construct
  REGION 1..119: note = Synthetic polypeptide
EVQLVQSGAEVKKPGASVKVSCKASGYKFTDSEMHWVRQAPGQGLEWIGGVDPETEGAAYNQKFKGRATITRDTSTSTAYLELSSLRSEDTAVYYCTRGYDYDYALDYWGQGTLVTVSS

SEQ ID NO: 117 - protein, 106 aa, synthetic construct
  REGION 1..106: note = Synthetic polypeptide
DIQMTQSPSSLSASVGDRVTITCRASSSVEFIHWYQQKPGKAPKPLISATSNLASGVPSRFSGSGSGTDFTLTISSLQPEDFATYYCQQWSSAPWTFGQGTKVEIK

SEQ ID NO: 118 - protein, 561 aa, synthetic construct
  REGION 1..561: note = Synthetic polypeptide
EVQLVQSGAEVKKPGASVKVSCKASGYKFTDSEMHWVRQAPGQGLEWIGGVDPETEGAAYNQKFKGRATITRDTSTSTAYLELSSLRSEDTAVYYCTRGYDYDYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKE

SEQ ID NO: 119 - protein, 213 aa, synthetic construct
  REGION 1..213: note = Synthetic polypeptide
DIQMTQSPSSLSASVGDRVTITCRASSSVEFIHWYQQKPGKAPKPLISATSNLASGVPSRFSGSGSGTDFTLTISSLQPEDFATYYCQQWSSAPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 231 aa, synthetic construct
  REGION 1..231: note = Synthetic polypeptide
EVQLVESGGGLVQPGGSLRLSCAASGYSITSDFAWNWVRQAPGKGLEWVGYISYSGTTSYNPSLKSRITISRDNSKNTFYLQMNSLRAEDTAVYYCARENYYGRSHVGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 121 - protein, 218 aa, synthetic construct
  REGION 1..218: note = Synthetic polypeptide
DIQMTQSPSSLSASVGDRVTITCRASASVDSYGNSFIHWYQQKPGKAPKLLIYRASDLESGVPSRFSGSGSGTDFTLTISSLQPEDFATYYCQQNYADPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 453 aa, synthetic construct
  REGION 1..453: note = Synthetic polypeptide
EVQLVESGGGLVQPGGSLRLSCAASGYSITSDFAWNWVRQAPGKGLEWVGYISYSGTTSYNPSLKSRITISRDNSKNTFYLQMNSLRAEDTAVYYCARENYYGRSHVGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 20 aa, synthetic construct
  REGION 1..20: note = Synthetic polypeptide
MFINIKSILWMCSTLIVTHA

SEQ ID NO: 124 - protein, 566 aa, synthetic construct
  REGION 1..566: note = Synthetic polypeptide
EVQLVESGGGLVQPGGSLRLSCAASGYSITSDFAWNWVRQAPGKGLEWVGYISYSGTTSYNPSLKSRITISRDNSKNTFYLQMNSLRAEDTAVYYCARENYYGRSHVGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSLHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKE